SEQUENCE LISTING (122 sequences)

SEQ ID NO: 1 - protein, 8 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
XAXAXAXA

SEQ ID NO: 2 - protein, 9 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
AXAXAXAXA

SEQ ID NO: 3 - protein, 10 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
XAXAXAXAXA

SEQ ID NO: 4 - protein, 11 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
AXAXAXAXAXA

SEQ ID NO: 5 - protein, 12 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
XAXAXAXAXAXA

SEQ ID NO: 6 - protein, 13 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
AXAXAXAXAXAXA

SEQ ID NO: 7 - protein, 14 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
XAXAXAXAXAXAXA

SEQ ID NO: 8 - protein, 15 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
AXAXAXAXAXAXAXA

SEQ ID NO: 9 - protein, 16 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
XAXAXAXAXAXAXAXA

SEQ ID NO: 10 - protein, 17 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
AXAXAXAXAXAXAXAXA

SEQ ID NO: 11 - protein, 18 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
XAXAXAXAXAXAXAXAXA

SEQ ID NO: 12 - protein, 19 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
AXAXAXAXAXAXAXAXAXA

SEQ ID NO: 13 - protein, 20 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
XAXAXAXAXAXAXAXAXAXA

SEQ ID NO: 14 - protein, 21 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
AXAXAXAXAXAXAXAXAXAXA

SEQ ID NO: 15 - protein, 22 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
XAXAXAXAXAXAXAXAXAXAXA

SEQ ID NO: 16 - protein, 23 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
  SITE 22: note = 2-aminoisobutyric acid
AXAXAXAXAXAXAXAXAXAXAXA

SEQ ID NO: 17 - protein, 24 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
  SITE 23: note = 2-aminoisobutyric acid
XAXAXAXAXAXAXAXAXAXAXAXA

SEQ ID NO: 18 - protein, 25 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
  SITE 22: note = 2-aminoisobutyric acid
  SITE 24: note = 2-aminoisobutyric acid
AXAXAXAXAXAXAXAXAXAXAXAXA

SEQ ID NO: 19 - protein, 26 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
  SITE 23: note = 2-aminoisobutyric acid
  SITE 25: note = 2-aminoisobutyric acid
XAXAXAXAXAXAXAXAXAXAXAXAXA

SEQ ID NO: 20 - protein, 8 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
  SITE 2: note = D-alanine
  SITE 4: note = D-alanine
  SITE 6: note = D-alanine
  SITE 8: note = D-alanine
XAXAXAXA

SEQ ID NO: 21 - protein, 9 aa, synthetic construct
  SITE 1: note = D-alanine
  SITE 3: note = D-alanine
  SITE 5: note = D-alanine
  SITE 7: note = D-alanine
  SITE 9: note = D-alanine
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
AXAXAXAXA

SEQ ID NO: 22 - protein, 10 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 2: note = D-alanine
  SITE 4: note = D-alanine
  SITE 6: note = D-alanine
  SITE 8: note = D-alanine
  SITE 10: note = D-alanine
XAXAXAXAXA

SEQ ID NO: 23 - protein, 11 aa, synthetic construct
  SITE 1: note = D-alanine
  SITE 3: note = D-alanine
  SITE 5: note = D-alanine
  SITE 7: note = D-alanine
  SITE 9: note = D-alanine
  SITE 11: note = D-alanine
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
AXAXAXAXAXA

SEQ ID NO: 24 - protein, 12 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 2: note = D-alanine
  SITE 4: note = D-alanine
  SITE 6: note = D-alanine
  SITE 8: note = D-alanine
  SITE 10: note = D-alanine
  SITE 12: note = D-alanine
XAXAXAXAXAXA

SEQ ID NO: 25 - protein, 13 aa, synthetic construct
  SITE 1: note = D-alanine
  SITE 3: note = D-alanine
  SITE 5: note = D-alanine
  SITE 7: note = D-alanine
  SITE 9: note = D-alanine
  SITE 11: note = D-alanine
  SITE 13: note = D-alanine
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
AXAXAXAXAXAXA

SEQ ID NO: 26 - protein, 14 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 2: note = D-alanine
  SITE 4: note = D-alanine
  SITE 6: note = D-alanine
  SITE 8: note = D-alanine
  SITE 10: note = D-alanine
  SITE 12: note = D-alanine
  SITE 14: note = D-alanine
XAXAXAXAXAXAXA

SEQ ID NO: 27 - protein, 15 aa, synthetic construct
  SITE 1: note = D-alanine
  SITE 3: note = D-alanine
  SITE 5: note = D-alanine
  SITE 7: note = D-alanine
  SITE 9: note = D-alanine
  SITE 11: note = D-alanine
  SITE 13: note = D-alanine
  SITE 15: note = D-alanine
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
AXAXAXAXAXAXAXA

SEQ ID NO: 28 - protein, 16 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 2: note = D-alanine
  SITE 4: note = D-alanine
  SITE 6: note = D-alanine
  SITE 8: note = D-alanine
  SITE 10: note = D-alanine
  SITE 12: note = D-alanine
  SITE 14: note = D-alanine
  SITE 16: note = D-alanine
XAXAXAXAXAXAXAXA

SEQ ID NO: 29 - protein, 17 aa, synthetic construct
  SITE 1: note = D-alanine
  SITE 3: note = D-alanine
  SITE 5: note = D-alanine
  SITE 7: note = D-alanine
  SITE 9: note = D-alanine
  SITE 11: note = D-alanine
  SITE 13: note = D-alanine
  SITE 15: note = D-alanine
  SITE 17: note = D-alanine
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
AXAXAXAXAXAXAXAXA

SEQ ID NO: 30 - protein, 18 aa, synthetic construct
  SITE 1: note = 2-aminoisobutyric acid
  STRAND 3: note = 2-aminoisobutyric acid
  SITE 5: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 2: note = D-alanine
  SITE 4: note = D-alanine
  SITE 6: note = D-alanine
  SITE 8: note = D-alanine
  SITE 10: note = D-alanine
  SITE 12: note = D-alanine
  SITE 14: note = D-alanine
  SITE 16: note = D-alanine
  SITE 18: note = D-alanine
XAXAXAXAXAXAXAXAXA

SEQ ID NO: 31 - protein, 19 aa, synthetic construct
  SITE 1: note = D-alanine
  SITE 3: note = D-alanine
  SITE 5: note = D-alanine
  SITE 7: note = D-alanine
  SITE 9: note = D-alanine
  SITE 11: note = D-alanine
  SITE 13: note = D-alanine
  SITE 15: note = D-alanine
  SITE 17: note = D-alanine
  SITE 19: note = D-alanine
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
AXAXAXAXAXAXAXAXAXA

SEQ ID NO: 32 - protein, 20 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 2: note = D-alanine
  SITE 4: note = D-alanine
  SITE 6: note = D-alanine
  SITE 8: note = D-alanine
  SITE 10: note = D-alanine
  SITE 12: note = D-alanine
  SITE 14: note = D-alanine
  SITE 16: note = D-alanine
  SITE 18: note = D-alanine
  SITE 20: note = D-alanine
XAXAXAXAXAXAXAXAXAXA

SEQ ID NO: 33 - protein, 21 aa, synthetic construct
  SITE 1: note = D-alanine
  SITE 3: note = D-alanine
  SITE 5: note = D-alanine
  SITE 7: note = D-alanine
  SITE 9: note = D-alanine
  SITE 11: note = D-alanine
  SITE 13: note = D-alanine
  SITE 15: note = D-alanine
  SITE 17: note = D-alanine
  SITE 19: note = D-alanine
  SITE 21: note = D-alanine
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 16: note = 2-aminoisobutyric acid
  SITE 14: note = 2-aminoisobutyric acid
  SITE 18: note = 2-aminoisobutyric acid
  SITE 20: note = 2-aminoisobutyric acid
AXAXAXAXAXAXAXAXAXAXA

SEQ ID NO: 34 - protein, 22 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
  SITE 2: note = D-alanine
  SITE 4: note = D-alanine
  SITE 6: note = D-alanine
  SITE 8: note = D-alanine
  SITE 10: note = D-alanine
  SITE 12: note = D-alanine
  SITE 14: note = D-alanine
  SITE 16: note = D-alanine
  SITE 18: note = D-alanine
  SITE 20: note = D-alanine
  SITE 22: note = D-alanine
XAXAXAXAXAXAXAXAXAXAXA

SEQ ID NO: 35 - protein, 23 aa, synthetic construct
  SITE 1: note = D-alanine
  SITE 3: note = D-alanine
  SITE 5: note = D-alanine
  SITE 7: note = D-alanine
  SITE 9: note = D-alanine
  SITE 11: note = D-alanine
  SITE 13: note = D-alanine
  SITE 15: note = D-alanine
  SITE 17: note = D-alanine
  SITE 19: note = D-alanine
  SITE 21: note = D-alanine
  SITE 23: note = D-alanine
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
  SITE 22: note = 2-aminoisobutyric acid
AXAXAXAXAXAXAXAXAXAXAXA

SEQ ID NO: 36 - protein, 24 aa, synthetic construct
  SITE 1: note = 2-aminoisobutyric acid
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
  SITE 23: note = 2-aminoisobutyric acid
  SITE 2: note = D-alanine
  SITE 4: note = D-alanine
  SITE 6: note = D-alanine
  SITE 8: note = D-alanine
  SITE 10: note = D-alanine
  SITE 12: note = D-alanine
  SITE 16: note = D-alanine
  SITE 14: note = D-alanine
  SITE 18: note = D-alanine
  SITE 20: note = D-alanine
  SITE 22: note = D-alanine
  SITE 24: note = D-alanine
XAXAXAXAXAXAXAXAXAXAXAXA

SEQ ID NO: 37 - protein, 17 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
AXAXAXAXAGRGDRGDG

SEQ ID NO: 38 - protein, 17 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
LXLXLXLXLGRGDRGDG

SEQ ID NO: 39 - protein, 23 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
AXAXAXAXAXAXAXAGRGDRGDG

SEQ ID NO: 40 - protein, 29 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
AXAXAXAXAXAXAXAXAXAXAGRGDRGDG

SEQ ID NO: 41 - protein, 32 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
AXAXAXAXAXAXAXAXAXAXAGRGDRGDRGDG

SEQ ID NO: 42 - protein, 24 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
AXAXAXAXAXAXAXAXAXAXAGSG

SEQ ID NO: 43 - protein, 26 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
AXAXAXAXAXAXAXAXAXAXAGSGSG

SEQ ID NO: 44 - protein, 28 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
AXAXAXAXAXAXAXAXAXAXAGSGSGSG

SEQ ID NO: 45 - protein, 30 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
AXAXAXAXAXAXAXAXAXAXAGSGSGSGSG

SEQ ID NO: 46 - protein, 8 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
AXAXAXAX

SEQ ID NO: 47 - protein, 10 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
AXAXAXAXAX

SEQ ID NO: 48 - protein, 12 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
AXAXAXAXAXAX

SEQ ID NO: 49 - protein, 14 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
AXAXAXAXAXAXAX

SEQ ID NO: 50 - protein, 16 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
AXAXAXAXAXAXAXAX

SEQ ID NO: 51 - protein, 18 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
AXAXAXAXAXAXAXAXAX

SEQ ID NO: 52 - protein, 10 aa, synthetic construct
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
RAXAXAXAXA

SEQ ID NO: 53 - protein, 16 aa, synthetic construct
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
RAXAXAXAXAXAXAXA

SEQ ID NO: 54 - protein, 22 aa, synthetic construct
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
RAXAXAXAXAXAXAXAXAXAXA

SEQ ID NO: 55 - protein, 23 aa, synthetic construct
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
  SITE 22: note = 2-aminoisobutyric acid
RRAXAXAXAXAXAXAXAXAXAXA

SEQ ID NO: 56 - protein, 24 aa, synthetic construct
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
  SITE 23: note = 2-aminoisobutyric acid
RRRAXAXAXAXAXAXAXAXAXAXA

SEQ ID NO: 57 - protein, 25 aa, synthetic construct
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
  SITE 22: note = 2-aminoisobutyric acid
  SITE 24: note = 2-aminoisobutyric acid
RRRRAXAXAXAXAXAXAXAXAXAXA

SEQ ID NO: 58 - protein, 31 aa, synthetic construct
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
RAXAXAXAXAXAXAXAXAXAXARGDRGDRGD

SEQ ID NO: 59 - protein, 34 aa, synthetic construct
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
RAXAXAXAXAXAXAXAXAXAXARGDRGDRGDRGD

SEQ ID NO: 60 - protein, 33 aa, synthetic construct
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
RAXAXAXAXAXAXAXAXAXAXAGRGDRGDRGDG

SEQ ID NO: 61 - protein, 36 aa, synthetic construct
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
RAXAXAXAXAXAXAXAXAXAXAGRGDRGDRGDRGDG

SEQ ID NO: 62 - protein, 34 aa, synthetic construct
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
  SITE 22: note = 2-aminoisobutyric acid
RRAXAXAXAXAXAXAXAXAXAXAGRGDRGDRGDG

SEQ ID NO: 63 - protein, 35 aa, synthetic construct
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
  SITE 23: note = 2-aminoisobutyric acid
RRRAXAXAXAXAXAXAXAXAXAXAGRGDRGDRGDG

SEQ ID NO: 64 - protein, 28 aa, synthetic construct
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
  SITE 22: note = 2-aminoisobutyric acid
  SITE 24: note = 2-aminoisobutyric acid
  SITE 26: note = 2-aminoisobutyric acid
RGDRGDAXAXAXAXAXAXAXAXAXAXAR

SEQ ID NO: 65 - protein, 31 aa, synthetic construct
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
  SITE 23: note = 2-aminoisobutyric acid
  SITE 25: note = 2-aminoisobutyric acid
  SITE 27: note = 2-aminoisobutyric acid
  SITE 29: note = 2-aminoisobutyric acid
RGDRGDRGDAXAXAXAXAXAXAXAXAXAXAR

SEQ ID NO: 66 - protein, 34 aa, synthetic construct
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
  SITE 22: note = 2-aminoisobutyric acid
  SITE 24: note = 2-aminoisobutyric acid
  SITE 26: note = 2-aminoisobutyric acid
  SITE 28: note = 2-aminoisobutyric acid
  SITE 30: note = 2-aminoisobutyric acid
  SITE 32: note = 2-aminoisobutyric acid
RGDRGDRGDRGDAXAXAXAXAXAXAXAXAXAXAR

SEQ ID NO: 67 - protein, 29 aa, synthetic construct
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
  SITE 22: note = 2-aminoisobutyric acid
  SITE 24: note = 2-aminoisobutyric acid
  SITE 26: note = 2-aminoisobutyric acid
RGDRGDAXAXAXAXAXAXAXAXAXAXARR

SEQ ID NO: 68 - protein, 32 aa, synthetic construct
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
  SITE 23: note = 2-aminoisobutyric acid
  SITE 25: note = 2-aminoisobutyric acid
  SITE 27: note = 2-aminoisobutyric acid
  SITE 29: note = 2-aminoisobutyric acid
RGDRGDRGDAXAXAXAXAXAXAXAXAXAXARR

SEQ ID NO: 69 - protein, 35 aa, synthetic construct
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
  SITE 22: note = 2-aminoisobutyric acid
  SITE 24: note = 2-aminoisobutyric acid
  SITE 26: note = 2-aminoisobutyric acid
  SITE 28: note = 2-aminoisobutyric acid
  SITE 30: note = 2-aminoisobutyric acid
  SITE 32: note = 2-aminoisobutyric acid
RGDRGDRGDRGDAXAXAXAXAXAXAXAXAXAXARR

SEQ ID NO: 70 - protein, 22 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
AXAXAXAXAXAXAXAXAXAXAR

SEQ ID NO: 71 - protein, 29 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
  SITE 22: note = 2-aminoisobutyric acid
  SITE 24: note = 2-aminoisobutyric acid
  SITE 26: note = 2-aminoisobutyric acid
AXAXAXAXAXAXAXAXAXAXAXAXAXARR

SEQ ID NO: 72 - protein, 35 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
  SITE 22: note = 2-aminoisobutyric acid
  SITE 24: note = 2-aminoisobutyric acid
  SITE 26: note = 2-aminoisobutyric acid
  SITE 26: note = 2-aminoisobutyric acid
  SITE 28: note = 2-aminoisobutyric acid
  SITE 30: note = 2-aminoisobutyric acid
  SITE 32: note = 2-aminoisobutyric acid
AXAXAXAXAXAXAXAXAXAXAXAXAXAXAXAXARR

SEQ ID NO: 73 - protein, 24 aa, synthetic construct
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
  SITE 22: note = 2-aminoisobutyric acid
GSAXAXAXAXAXAXAXAXAXAXAR

SEQ ID NO: 74 - protein, 26 aa, synthetic construct
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
  SITE 22: note = 2-aminoisobutyric acid
  SITE 24: note = 2-aminoisobutyric acid
GSGSAXAXAXAXAXAXAXAXAXAXAR

SEQ ID NO: 75 - protein, 28 aa, synthetic construct
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
  SITE 22: note = 2-aminoisobutyric acid
  SITE 24: note = 2-aminoisobutyric acid
  SITE 26: note = 2-aminoisobutyric acid
GSGSGSAXAXAXAXAXAXAXAXAXAXAR

SEQ ID NO: 76 - protein, 30 aa, synthetic construct
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
  SITE 22: note = 2-aminoisobutyric acid
  SITE 24: note = 2-aminoisobutyric acid
  SITE 26: note = 2-aminoisobutyric acid
  SITE 28: note = 2-aminoisobutyric acid
GSGSGSGSAXAXAXAXAXAXAXAXAXAXAR

SEQ ID NO: 77 - protein, 22 aa, synthetic construct
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
EAXAXAXAXAXAXAXAXAXAXA

SEQ ID NO: 78 - protein, 23 aa, synthetic construct
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 22: note = 2-aminoisobutyric acid
  SITE 20: note = 2-aminoisobutyric acid
EEAXAXAXAXAXAXAXAXAXAXA

SEQ ID NO: 79 - protein, 24 aa, synthetic construct
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 17: note = 2-aminoisobutyric acid
  SITE 19: note = 2-aminoisobutyric acid
  SITE 21: note = 2-aminoisobutyric acid
  SITE 23: note = 2-aminoisobutyric acid
EEEAXAXAXAXAXAXAXAXAXAXA

SEQ ID NO: 80 - protein, 25 aa, synthetic construct
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 20: note = 2-aminoisobutyric acid
  SITE 18: note = 2-aminoisobutyric acid
  SITE 22: note = 2-aminoisobutyric acid
  SITE 24: note = 2-aminoisobutyric acid
EEEEAXAXAXAXAXAXAXAXAXAXA

SEQ ID NO: 81 - protein, 31 aa, synthetic construct
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
EAXAXAXAXAXAXAXAXAXAXARGDRGDRGD

SEQ ID NO: 82 - protein, 30 aa, synthetic construct
  SITE 3: note = 2-aminoisobutyric acid
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
EAXAXAXAXAXAXAXAXAXAXAGSGSGSGS

SEQ ID NO: 83 - protein, 22 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
AXAXAXAXAXAXAXAXAXAXAE

SEQ ID NO: 84 - protein, 23 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
AXAXAXAXAXAXAXAXAXAXAEE

SEQ ID NO: 85 - protein, 30 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  REGION 1..30: note = Among the entire sequence, amino acids 1 and 30 may be conjugated with each other to form a cyclic peptide
AXAXAXAXGHHHHHHHHHHGRGDRGDRGDG

SEQ ID NO: 86 - protein, 32 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  REGION 1..32: note = Among the entire sequence, amino acids 1 and 32 may be conjugated with each other to form a cyclic peptide
AXAXAXAXAXGHHHHHHHHHHGRGDRGDRGDG

SEQ ID NO: 87 - protein, 34 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  REGION 1..34: note = Among the entire sequence, amino acids 1 and 34 may be conjugated with each other to form a cyclic peptide
AXAXAXAXAXAXGHHHHHHHHHHGRGDRGDRGDG

SEQ ID NO: 88 - protein, 36 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  REGION 1..36: note = Among the entire sequence, amino acids 1 and 36 may be conjugated with each other to form a cyclic peptide
AXAXAXAXAXAXAXGHHHHHHHHHHGRGDRGDRGDG

SEQ ID NO: 89 - protein, 38 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 16: note = 2-aminoisobutyric acid
  SITE 14: note = 2-aminoisobutyric acid
  REGION 1..38: note = Among the entire sequence, amino acids 1 and 38 may be conjugated with each other to form a cyclic peptide
AXAXAXAXAXAXAXAXGHHHHHHHHHHGRGDRGDRGDG

SEQ ID NO: 90 - protein, 40 aa, synthetic construct
  SITE 2: note = 2-aminoisobutyric acid
  SITE 4: note = 2-aminoisobutyric acid
  SITE 6: note = 2-aminoisobutyric acid
  SITE 8: note = 2-aminoisobutyric acid
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  REGION 1..40: note = Among the entire sequence, amino acids 1 and 40 may be conjugated with each other to form a cyclic peptide
AXAXAXAXAXAXAXAXAXGHHHHHHHHHHGRGDRGDRGDG

SEQ ID NO: 91 - protein, 37 aa, synthetic construct
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
  SITE 23: note = 2-aminoisobutyric acid
  SITE 25: note = 2-aminoisobutyric acid
  SITE 27: note = 2-aminoisobutyric acid
  SITE 29: note = 2-aminoisobutyric acid
  SITE 31: note = 2-aminoisobutyric acid
  REGION 1..37: note = Among the entire sequence, amino acids 1 and 37 may be conjugated with each other to form a cyclic peptide
GRGDRGDRGDGAXAXAXAXAXAXAXAXAXAXAGRGDG

SEQ ID NO: 92 - protein, 37 aa, synthetic construct
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
  SITE 23: note = 2-aminoisobutyric acid
  SITE 25: note = 2-aminoisobutyric acid
GRGDGAXAXAXAXAXAXAXAXAXAXAGRGDRGDRGDG

SEQ ID NO: 93 - protein, 40 aa, synthetic construct
  SITE 10: note = 2-aminoisobutyric acid
  SITE 12: note = 2-aminoisobutyric acid
  SITE 14: note = 2-aminoisobutyric acid
  SITE 16: note = 2-aminoisobutyric acid
  SITE 18: note = 2-aminoisobutyric acid
  SITE 20: note = 2-aminoisobutyric acid
  SITE 22: note = 2-aminoisobutyric acid
  SITE 24: note = 2-aminoisobutyric acid
  SITE 26: note = 2-aminoisobutyric acid
  SITE 28: note = 2-aminoisobutyric acid
GRGDRGDGAXAXAXAXAXAXAXAXAXAXAGRGDRGDRGDG

SEQ ID NO: 94 - protein, 43 aa, synthetic construct
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
  SITE 23: note = 2-aminoisobutyric acid
  SITE 25: note = 2-aminoisobutyric acid
  SITE 27: note = 2-aminoisobutyric acid
  SITE 29: note = 2-aminoisobutyric acid
  SITE 31: note = 2-aminoisobutyric acid
GRGDRGDRGDGAXAXAXAXAXAXAXAXAXAXAGRGDRGDRGDG

SEQ ID NO: 95 - protein, 27 aa, synthetic construct
  SITE 5: note = 2-aminoisobutyric acid
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
GSGAXAXAXAXAXAXAXAXAXAXAGSG

SEQ ID NO: 96 - protein, 31 aa, synthetic construct
  SITE 7: note = 2-aminoisobutyric acid
  SITE 9: note = 2-aminoisobutyric acid
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
  SITE 23: note = 2-aminoisobutyric acid
GSGSGAXAXAXAXAXAXAXAXAXAXAGSGSG

SEQ ID NO: 97 - protein, 39 aa, synthetic construct
  SITE 11: note = 2-aminoisobutyric acid
  SITE 13: note = 2-aminoisobutyric acid
  SITE 15: note = 2-aminoisobutyric acid
  SITE 17: note = 2-aminoisobutyric acid
  SITE 19: note = 2-aminoisobutyric acid
  SITE 21: note = 2-aminoisobutyric acid
  SITE 23: note = 2-aminoisobutyric acid
  SITE 25: note = 2-aminoisobutyric acid
  SITE 27: note = 2-aminoisobutyric acid
  SITE 29: note = 2-aminoisobutyric acid
GSGSGSGSGAXAXAXAXAXAXAXAXAXAXAGSGSGSGSG

SEQ ID NO: 98 - protein, 6 aa, synthetic construct
RGDRGD

SEQ ID NO: 99 - protein, 9 aa, synthetic construct
RGDRGDRGD

SEQ ID NO: 100 - protein, 12 aa, synthetic construct
RGDRGDRGDRGD

SEQ ID NO: 101 - skipped

SEQ ID NO: 102 - protein, 5 aa, synthetic construct
GSGSG

SEQ ID NO: 103 - protein, 7 aa, synthetic construct
GSGSGSG

SEQ ID NO: 104 - protein, 9 aa, synthetic construct
GSGSGSGSG

SEQ ID NO: 105 - protein, 23 aa, synthetic construct
GHHHHHHHHHHHGRGDRGDRGDG

SEQ ID NO: 106 - DNA, 21 bp, synthetic construct
  source 1..21: mol_type = other DNA
acggagactggagtcgaagag

SEQ ID NO: 107 - DNA, 23 bp, synthetic construct
  source 1..23: mol_type = other DNA
gtagggcaactagtgcatctccc

SEQ ID NO: 108-110 - skipped (3 entries)

SEQ ID NO: 111 - protein, 4 aa, synthetic construct
RRRR

SEQ ID NO: 112 - protein, 5 aa, synthetic construct
RRRRR

SEQ ID NO: 113-115 - skipped (3 entries)

SEQ ID NO: 116 - protein, 4 aa, synthetic construct
HHHH

SEQ ID NO: 117 - protein, 5 aa, synthetic construct
HHHHH

SEQ ID NO: 118-119 - skipped (2 entries)

SEQ ID NO: 120 - DNA, 20 bp, synthetic construct
  source 1..20: mol_type = other DNA
ccaaacctaagactgccgtc

SEQ ID NO: 121-122 - skipped (2 entries)

(19) United States
(12) Patent Application Publication
(10) Pub. No.: US 2024/0424134 A1
(43) Pub. Date: Dec. 26, 2024

(54) PEPTIDE HAVING INDEPENDENTLY STABILIZED HYDROPHOBIC ALPHA HELIX, PEPTIDE COMPLEX COMPRISING SAME AND USES THEREOF

(71) Applicant: UIF (University Industry Foundation), Yonsei University, Seoul (KR)

(72) Inventors: Yong-beom LIM, Seoul (KR); You-Jin JUNG, Seoul (KR); Hyoseok KIM, Seoul (KR)

(73) Assignee: UIF (University Industry Foundation), Yonsei University, Seoul (KR)

(21) Appl. No.: 18/517,548

(22) Filed: Nov. 22, 2023

(30) Foreign Application Priority Data
Nov. 29, 2022 (KR) 10-2022-0162494

(51) Int. Cl. A61K 48/00; A61K 31/7105; A61K 31/711; A61K 41/00; C07K 1/107; C07K 7/06; C07K 7/08; C12N 15/113

(52) U.S. Cl. CPC A61K 48/0041; A61K 31/7105; A61K 31/711; A61K 41/00; C07K 1/1077; C07K 7/06; C07K 7/08; C12N 15/113; C12N 2310/11; C12N 2310/12; C12N 2310/127; C12N 2310/14; C12N 2310/16

(57) ABSTRACT

The present disclosure relates to a novel hydrophobic peptide consisting of short amino acid sequences as repeat units, having a perfectly stabilized α-helix structure without help from outside. The peptide has no cytotoxicity and has magnetic responsiveness, i.e., its arrangement and orientation are controlled by a magnetic field. In addition, the hydrophobic peptide according to the present disclosure can not only form complexes of various structures with hydrophilic molecules but also provide suitable nanostructures such as nanoribbons and artificial chromosome-like structures through stepwise association with nucleic acid materials.

### CROSS-REFERENCE TO RELATED APPLICATION

This application claims priority to Korean Patent Application No. 10-2022-0162494 filed on Nov. 29, 2022, and all the benefits accruing therefrom under 35 U.S.C. § 119, the contents of which in its entirety are herein incorporated by reference.

### SEQUENCE LISTING

The instant application contains a Sequence Listing which has been submitted electronically in a computer readable Sequence Listing XML format and is hereby incorporated by reference in its entirety. Said computer readable Sequence Listing in XML format was created on Nov. 22, 2023, is named G1035-26201_SequenceListing.xml and is 472,398 bytes in size.

### BACKGROUND

### 1. Field

The present disclosure relates to a peptide having an independently stabilized hydrophobic α-helix structure without help from outside, a peptide complex including the same, and a use thereof.

### 2. Description of the Related Art

Magnetically responsive materials are highly valuable for biological and medical uses because the magnetic field is radiation-free and safe for humans. A variety of stimuli-responsive self-assembled materials have been developed to date. Although magnetically responsive self-assembled materials based on ferromagnetic/paramagnetic inorganic materials or composites of inorganic and organic materials have been developed, magnetically responsive self-assembled materials based solely on organic materials have not been reported. DNAs and some proteins have been shown to be magnetically responsive. However, there is difficulty in actual commercialization since an extremely strong magnetic field is required.

Self-assembled peptide structures are receiving a lot of attentions due to their potential for various biological applications such as medical or nanotechnological fields. The self-assembled peptide structures have inherently high biological stability because they are made up of short units. These peptide monomers can form secondary structures such as α-helix or β-sheet and can form various structures such as micelles, vesicles, nanofibers, hydrogels, etc. through hydrophobic bonds, hydrogen bonds, electrostatic attractions, π-π bonds, etc.

As such, peptides have attracted much attention as they can realize more precise and stable functional structures by providing not only various structures but also various functions depending on their properties. However, as representative diamagnetic organic molecules, they have been regarded as non-magnetic materials since they are not sensitive to strong magnetic fields of several teslas or higher.

Recently, it was found out that the α-helix structure, which is the secondary structure of peptide monomers, has slight magnetic responsiveness. However, there is a problem that the peptides with an α-helix structure do not maintain the α-helix structure in the monomer state or have the α-helix structure only when they exist in a protein.

Regarding magnetically responsive peptides, a one-dimensional nanostructure formed through self-assembly of dipeptides whose arrangement can be controlled with a magnetic field or an electric field is disclosed in the patent document 1. However, since the structure is limited in organic solvents and the peptide sequence is limited to Phe-Phe, wide application is impossible and an additional modification process is necessary to provide chemical and biological functions.

That is to say, although it should be possible to align the magnetic responsiveness along the direction of the magnetic field while forming a rod-shaped self-assembled peptide structure in order to use diamagnetic organic materials such as peptides as magnetically responsive organic materials capable of controlling the mechanical movement using magnetic force as a noninvasive stimulation source, related researches are very insufficient until now. In addition, although it is very important to maintain the secondary structure of the peptide stably for wide application through interaction with genetic materials, there are few related researches and no research has been conducted on the control of the formation and disassembly of self-assembled peptides.

### References of the Related Art

Patent document 1. Korean Patent Publication No. 10-2008-0102687.

### SUMMARY

The present disclosure is directed to providing a new peptide having an independently stabilized hydrophobic α-helix structure without help from outside and capable of forming various self-assembled structures through binding to hydrophilic molecules.

The present disclosure is also directed to providing a peptide complex including the hydrophobic peptide.

The present disclosure is also directed to providing a new system capable of safely storing nucleic acid information.

The present disclosure provides a hydrophobic peptide represented by General Formula 1 or 2:

-{[Aib]-[Xaa1]}ₘ- [General Formula 1]

Xaa1-{[Aib]-[Xaa1]}m- [General Formula 2]
wherein
Xaa1 is a D- or L-amino acid residue selected from a group consisting of Ala (A), Ile (I), Leu (L), Met (M) and Val (V), and
m is any integer selected from 4 to 50.

In General Formula 1, Xaa1 may be any one selected from a group consisting of Ala (A), Ile (I), Leu (L) and Met (M).

In General Formula 1, Xaa1 may be Ala (A) or Leu (L).

In General Formula 1, m may be any integer selected from 6 to 13.

In General Formula 1, m may be any integer selected from 6 to 10.

The hydrophobic peptide may be a rod-shaped hydrophobic peptide having an α-helix structure.

The hydrophobic peptide may be controlled to be arranged and oriented in the direction of an external magnetic field.

The present disclosure also provides a peptide complex including: the hydrophobic peptide; and at least one hydrophilic polymer or hydrophilic peptide bound to one or both ends of the hydrophobic peptide.

The hydrophilic polymer may be any one selected from a group consisting of polyethylene glycol (PEG), poly(N-(2-hydroxypropyl)methacrylamide), poly(2-(methacryloyloxy)ethyl phosphorylcholine), poly(hydroxyalkyl L-asparagine) and poly(hydroxyalkyl L-glutamine).

The hydrophilic peptide may consist of 3-20 amino acid sequences, wherein 70-100% of the amino acid sequences are hydrophilic amino acids, and the hydrophilic amino acid may be one or more selected from a group consisting of H (histidine), N (asparagine), Q (glutamine), S (serine), T (threonine), C (cysteine), G (glycine), K (lysine), R (arginine), E (glutamic acid) and D (aspartic acid).

The hydrophilic peptide may be any one selected from a group consisting of SEQ ID NOS 98-105.

The peptide complex may be cyclic with both ends of the peptide complex linked.

The peptide complex may further include a positively charged peptide consisting of positively charged 1-4 amino acid residues at the N- or C-terminal.

The positively charged peptide may be any one selected from SEQ ID NOS 108-119.

The peptide complex may self-assemble into a spherical nanoparticle such as a micellar structure or a vesicular structure in solution.

The peptide complex may self-assemble into an anisotropic planar nanostructure or a cruciform anisotropic nanostructure under the condition where a magnetic field is applied.

The intensity of the magnetic field may be 0.1-2 T.

The present disclosure also provides a composition for safely storing nucleic acid information, which contains: the peptide complex; and a nucleic acid material.

The peptide complex and the nucleic acid material may self-assemble into a nanoribbon or a nanostructure having an artificial chromosome-like structure via noncovalent bonding.

A nanoribbon structure may be formed through self-assembly as the peptide complex binds to the nucleic acid material and a nanostructure with an artificial chromosome-like structure may be formed through self-assembly as the nanoribbon is folded and stacked.

The nucleic acid material may be one or more selected from a group consisting of an RNA, a DNA, an siRNA (short interfering RNA), an aptamer, an antisense ODN (oligodeoxynucleotide), an antisense RNA, a ribozyme and a DNAzyme.

The nanostructure may be degraded at a magnetic field intensity of 0.1-2 T and induce the release of the nucleic acid material.

The magnetic field intensity may be 0.1-0.5 T based on a rotating magnetic field.

The present disclosure relates to a novel hydrophobic peptide consisting of short amino acid sequences as repeat units, having a perfectly stabilized α-helix structure without help from outside. The peptide has no cytotoxicity and has magnetic responsiveness, i.e., its arrangement and orientation are controlled by a magnetic field. In addition, the hydrophobic peptide according to the present disclosure can not only form complexes of various structures with hydrophilic molecules but also provide suitable nanostructures such as nanoribbons and artificial chromosome-like structures through stepwise association with nucleic acid materials.

A peptide complex including the hydrophobic peptide according to the present disclosure can provide a magnetically responsive material with a new structure that can completely protect nucleic acid materials from external environment and the assembly and degradation of which can be controlled with a magnetic field.

Since the peptide complex according to the present disclosure forms a nanostructure by associating with a nucleic acid material and can be applied directly to analysis methods such as PCR for identifying the genetic information of the nucleic acid material, it can be usefully used in various drug delivery systems, drug therapy technologies, cosmetic compositions, molecular machines, etc.

### BRIEF DESCRIPTION OF THE DRAWINGS

FIGS. 1-36 show a result of analyzing hydrophobic peptides of Examples 1-36 by MALDI-TOF.

FIG. 37 shows the structure of peptide complexes prepared in Examples 37-40, 125 and 128.

FIG. 38 shows the structure of peptide complexes prepared in Examples 46-50.

FIG. 39 shows the structure of cyclic peptide complexes prepared in Examples 98-102.

FIG. 40 shows the MALDI-TOF mass spectrometry result for peptide complexes prepared in Examples 37, 39, 40, 46-50, 60, 98-102, 125 and 128.

FIG. 41 shows the HPLC chromatograms of peptide complexes prepared in Examples 37, 39, 40, 46-50, 60, 98-102, 125 and 128.

FIG. 42A schematically shows the diamagnetic anisotropy of the peptide bond of a hydrophobic peptide according to the present disclosure, wherein B₀ represents external magnetic field (MF). FIG. 42B shows the α-helix structure and helical axis of the hydrophobic peptide according to the present disclosure. The aligned peptide bonds are shown and the difference in the diamagnetic susceptibility between the parallel and perpendicular directions is shown schematically.

FIG. 42C shows the rod-coil structure of peptide complexes prepared in Examples 37-41 (αₘ-RGD). The peptide complex according to the present disclosure has a rod-coil structure formed as a hydrophobic peptide including a repeat unit (a) consisting of two amino acid residues binds to a hydrophilic peptide (RGD). FIG. 42D shows the CD spectra of the peptide complexes prepared in Examples 37, 39 and 40 (α₄-RGD₂, α₇-RGD₂ and α₁₀-RGD₂).

FIG. 42E shows the structure of a peptide complex prepared in Example 125 (PEG₁₆-α₁₀-PEG₁₆). FIG. 42F shows the CD spectra of the peptide complex prepared in Example 125 depending on concentration and FIG. 42G shows a result of measuring the helicity of the peptide complex prepared in Example 125 depending on concentration.

FIG. 43 shows AFM images of peptide complexes prepared in Examples 37, 39, 40 and 125 (a, b, c, d; α₄-(RGD)₂, α₇-(RGD)₂, α₁₀-(RGD)₂, PEG₁₆-α₁₀-PEG₁₆) for comparing the self-assembly behavior of a peptide complex having a rod-coil structure and a peptide complex having a coil-rod-coil structure.

FIG. 44A shows the ¹H-¹⁵N IPAP-HSQC spectrum of a peptide complex prepared in Example 128 (PEG₃₀-α₁₀-PEG₃₀) and FIG. 44B schematically shows the behavior of the peptide complex prepared in Example 128 (PEG₃₀-α₁₀-PEG₃₀) under a magnetic field. FIG. 44C schematically shows the difference in the chemical structure of a peptide complex of Example 47 and a peptide complex of Example 99. FIG. 44D shows the CD and MCD (magnetic circular dichroism) spectra of the peptide complex of Example 47 (L-α₅-PEG₁₀). FIG. 44E shows the CD and MCD (magnetic circular dichroism) spectra of the peptide complex of Example 99 (C-α₅-PEG₁₀).

FIG. 44F shows a peptide complex (specimen) dispersed in an aqueous solvent placed between neodymium magnets and FIG. 44G shows the AFM images of the peptide complex of Example 99 (C-α₅-PEG₁₀) in the presence (right) or absence (left) of a magnetic field magnetic field. FIG. 44H shows the AFM images of a peptide complex of Example 51 (α₁₀-(RGD)₃) in the presence (right) or absence (left) of a magnetic field. FIG. 44I schematically shows a model of a self-assembly processes in the presence (with) or absence (without) of a magnetic field.

FIG. 45 shows the ¹H NMR spectrum of a peptide complex prepared in Example 128 (PEG₃₀-α₁₀-PEG₃₀) measured at 700.400 MHz (16.45 T) and FIG. 46 shows the ¹H-¹⁵N IPAP-HSQC spectrum of the peptide complex prepared in Example 128 (PEG₃₀-α₁₀-PEG₃₀) measured at 700.400 MHz (16.45 T).

FIG. 47 shows the ¹H NMR spectrum of the peptide complex prepared in Example 128 (PEG₃₀-α₁₀-PEG₃₀) measured at 900.230 MHz (21.14 T) and FIG. 48 shows the ¹H-¹⁵N IPAP-HSQC spectrum of the peptide complex prepared in Example 128 (PEG₃₀-α₁₀-PEG₃₀) measured at 900.230 MHz (21.14 T).

FIG. 49A shows the structure of a peptide complex of Example 60 (R-α₁₀-PEG₁₆). In the peptide complex, the N-terminal of a hydrophobic peptide has two positive charges, one derived from the arginine residue and the other from the N-terminal amine. FIG. 49B shows the AFM image of a linear plasmid DNA (7.5 kb) and FIG. 49C shows the AFM image of the peptide complex of Example 60 (R-α₁₀-PEG₁₆).

FIG. 49D shows an EMSA (electrophoretic mobility shift assay) result for a peptide-DNA nanostructure prepared by mixing a linear plasmid DNA (7.5 kb) with the peptide complex of Example 60 (R-α₁₀-PEG₁₆) at different concentrations.

FIG. 49E to 49H show a process of forming a peptide-DNA nanostructure by mixing a linear plasmid DNA (7.5 kb) with the peptide complex of Example 60 (R-α₁₀-PEG₁₆) and a result of monitoring the morphology transformation of the nanostructure by AFM and TEM. FIG. 49E shows the AFM image of the peptide-DNA nanostructure in the early stage, FIG. 49F shows the AFM image of the peptide-DNA nanostructure in the intermediate stage, FIG. 49G shows the TEM image of the peptide-DNA nanostructure in the intermediate-to-late stage, FIG. 49H shows the TEM image of the peptide-DNA nanostructure in the late stage and FIG. 49I shows the mechanism whereby the peptide-DNA nanostructure is formed into a nanoribbon structure.

FIG. 50 shows the cleavage map of a plasmid DNA used in the present disclosure, which was cleaved at the Bam HI site. FIG. 51 shows the CD spectra of a peptide-DNA nanostructure of the peptide complex of Example 60 and a linear plasmid DNA and FIG. 52A shows the SAED (selected area electron diffraction) pattern of a peptide-DNA nanostructure in nanoribbon state and FIG. 52B shows a peptide-DNA nanostructure in artificial chromosome state. FIG. 53 shows the WAXS (synchrotron wide-angle X-ray scattering) analysis result of a peptide-DNA nanostructure of the peptide complex of Example 60 and a linear plasmid DNA.

FIG. 54A shows the CD spectrum of a peptide-DNA nanostructure (artificial chromosome form).

FIG. 54B shows the electrophoresis result of a peptide-DNA nanostructure (artificial chromosome form) in the presence of DNase 1. Ladder indicates a DNA size marker, DNA indicates the linear plasmid DNA, Complex indicates the peptide-DNA nanostructure (artificial chromosome form) and DNase indicates DNase 1. The arrow indicates the peptide-DNA nanostructures (artificial chromosome form) trapped in the lanes 4 and 5.

FIG. 54C shows a result of adding a peptide-DNA nanostructure (artificial chromosome form) of the peptide complex of Example 60 and a linear plasmid DNA in the presence of DNase I and analyzing its degradation with time (0-600 seconds) by electrophoresis.

FIG. 54D shows the PCR analysis result of a peptide-DNA nanostructure (artificial chromosome form) of the peptide complex of Example 60 and a linear plasmid DNA. Lane 1 indicates a DNA size marker, lane 2 indicates a PCR-amplified linear plasmid DNA and lane 3 indicates a PCR-amplified DNA from the peptide-DNA nanostructure (artificial chromosome form).

FIG. 55A shows the TEM image of a peptide-DNA nanostructure (nanoribbon form) of the peptide complex of Example 60 and a linear plasmid DNA before exposure to a magnetic field and FIG. 55B shows experimental equipment for generating an RMF (rotating magnetic field) from permanent magnets.

FIG. 55C shows the TEM images of a peptide-DNA nanostructure (nanoribbon form) of the peptide complex of Example 60 and a linear plasmid DNA after exposure to a rotating magnetic field.

FIG. 56 shows the AFM image of a peptide-DNA nanostructure (nanoribbon form) of the peptide complex of Example 60 and a linear plasmid DNA after exposure to a static magnetic field (0.1 T) for 2 weeks.

FIG. 57A shows the HPLC analysis result of a peptide complex of Example 37 (1.286 mM) and FIG. 57B shows the MALDI-TOF mass spectrometry analysis result of the peptide complex of Example 37 (1.286 mM).

FIG. 58A shows the HPLC analysis result of a peptide complex of Example 38 and FIG. 58B shows the MALDI-TOF mass spectrometry analysis result of the peptide complex of Example 38.

FIG. 59A shows the HPLC analysis result of a peptide complex of Example 39 and FIG. 59B shows the MALDI-TOF mass spectrometry analysis result of the peptide complex of Example 39.

FIG. 60A shows the HPLC analysis result of a peptide complex of Example 40 and FIG. 60B shows the MALDI-TOF mass spectrometry analysis result of the peptide complex of Example 40.

FIG. 61 shows the HPLC analysis result of a peptide complex of Example 53.

FIG. 62A shows the HPLC analysis result of peptide complexes of Examples 60-63 and FIG. 62B shows the MALDI-TOF mass spectrometry analysis result of the peptide complexes of Examples 60-63. In the graphs, R1 indicates the peptide complex of Example 60, R2 indicates the peptide complex of Example 61, R3 indicates the peptide complex of Example 62 and R4 indicates the peptide complex of Example 63.

FIG. 63A shows the HPLC analysis result of a peptide complex of Example 90 and FIG. 63B shows the MALDI-TOF mass spectrometry analysis result of the peptide complex of Example 90.

FIG. 64A shows the HPLC analysis result of a peptide complex of Example 91 and FIG. 64B shows the MALDI-TOF mass spectrometry analysis result of the peptide complex of Example 91.

FIG. 65A shows the HPLC analysis result of a peptide complex of Example 71 and FIG. 65B shows the MALDI-TOF mass spectrometry analysis result of the peptide complex of Example 71.

FIG. 66 shows the MALDI-TOF mass spectrometry analysis result of a peptide complex of Example 72.

FIG. 67A shows the HPLC analysis result of a peptide complex of Example 66 and FIG. 67B shows the MALDI-TOF mass spectrometry analysis result of the peptide complex of Example 66.

FIG. 68A shows the HPLC analysis result of a peptide complex of Example 67 and FIG. 68B shows the MALDI-TOF mass spectrometry analysis result of the peptide complex of Example 67.

FIG. 69A shows the HPLC analysis result of a peptide complex of Example 52 and FIG. 69B shows the MALDI-TOF mass spectrometry analysis result of the peptide complex of Example 52.

FIG. 70 shows the MALDI-TOF mass spectrometry analysis result of a peptide complex of Example 53.

FIG. 71A shows the HPLC analysis result of a peptide complex of Example 46 and FIG. 71B shows the MALDI-TOF mass spectrometry analysis result of the peptide complex of Example 46.

FIG. 72A shows the HPLC analysis result of a peptide complex of Example 47 and FIG. 72B shows the MALDI-TOF mass spectrometry analysis result of the peptide complex of Example 47.

FIG. 73A shows the HPLC analysis result of a peptide complex of Example 48 and FIG. 73B shows the MALDI-TOF mass spectrometry analysis result of the peptide complex of Example 48.

FIG. 74A shows the HPLC analysis result of a peptide complex of Example 49 and FIG. 74B shows the MALDI-TOF mass spectrometry analysis result of the peptide complex of Example 49.

FIG. 75A shows the HPLC analysis result of a peptide complex of Example 50 and FIG. 75B shows the MALDI-TOF mass spectrometry analysis result of the peptide complex of Example 50.

FIG. 76A shows the HPLC analysis result of a peptide complex of Example 51 and FIG. 76B shows the MALDI-TOF mass spectrometry analysis result of the peptide complex of Example 51.

FIG. 77 shows the CD spectra of peptide complexes of Examples 37 (a), 39 (b) and 40 (c).

FIG. 78 shows the CD spectrum of a peptide complex of Example 38.

FIG. 79 shows the CD spectrum of a peptide complex of Example 40.

FIG. 80A shows the AFM images of peptide complexes of Example 37. FIG. 80B shows the AFM images of peptide complexes of Example 38. FIG. 80C shows the AFM images of peptide complexes of Example 39. FIG. 80D shows the AFM images of peptide complexes of Example 40.

FIG. 81A shows the AFM image of a peptide complex of Example 60 (R-α₁₀-PEG₁₆) and FIG. 81B shows the AFM image of dsDNA.

FIGS. 81C and 81D show the AFM images of an R-α₁₀-PEG₁₆/dsDNA nanostructure obtained 2-3 minutes after preparation, FIG. 81E shows the AFM image of the R-α₁₀-PEG₁₆/dsDNA nanostructure obtained 12 days after the preparation and FIG. 81F shows the AFM image of the R-α₁₀-PEG₁₆/dsDNA nanostructure obtained 22 days after the preparation.

FIG. 82A shows the TEM image of an R-α₁₀-PEG₁₆/dsDNA nanostructure obtained 2-3 minutes after preparation, FIG. 82B shows the TEM image of the R-α₁₀-PEG₁₆/dsDNA nanostructure obtained 12 days after the preparation and FIGS. 82C and 82D show the TEM image of the R-α₁₀-PEG₁₆/dsDNA nanostructure obtained 22 days after the preparation.

FIG. 83 shows the CD spectra of a peptide complex of Example 60 (R-α₁₀-PEG₁₆), dsDNA and an R-α₁₀-PEG₁₆/dsDNA nanostructure.

FIG. 84 shows the SAED (selected area electron diffraction) pattern of an R-α₁₀-PEG₁₆/dsDNA nanostructure having a long nanoribbon structure and FIG. 85 shows the SAED (selected area electron diffraction) pattern of an R-α₁₀-PEG₁₆/dsDNA nanostructure having an artificial chromosome structure.

FIG. 86 shows a UV-vis measurement result obtained after adding DNase I to dsDNA and an R-α₁₀-PEG₁₆/dsDNA nanostructure.

FIG. 87 shows the AFM image of a peptide complex of Example 63, FIG. 88 shows the AFM image of an R₄-α₁₀-PEG₁₆/dsDNA nanostructure obtained 40 minutes after preparation and FIG. 89 shows the AFM image of the R₄-α₁₀-PEG₁₆/dsDNA nanostructure obtained 111 days after the preparation.

FIG. 90 shows the CD spectrum of an R₄-α₁₀-PEG₁₆/dsDNA nanostructure.

FIG. 91A shows the EMSA result for a nanostructure of a peptide complex of Example 60 (R-α₁₀-PEG₁₆) and dsDNA and FIG. 91B shows the EMSA result for a nanostructure of the peptide complex of Example 60 (R-α₁₀-PEG₁₆) and ssDNA.

FIG. 91C shows the EMSA result for a nanostructure of a peptide complex of Example 61 (R-α₁₀-PEG₁₆) and pDNA, FIG. 91D shows the EMSA result for a nanostructure of the peptide complex of Example 61 (R-α₁₀-PEG₁₆) and dsDNA, FIG. 91E shows the EMSA result for a nanostructure of a peptide complex of Example 62 (R-α₁₀-PEG₁₆) and dsDNA and FIG. 91F shows the EMSA result for a nanostructure of a peptide complex of Example 63 (R-α₁₀-PEG₁₆) and dsDNA.

FIG. 92A shows the HPLC result of Example 98. FIG. 92B shows the MALDI-TOF result of Example 98. FIG. 92C shows the CD result of Example 98.

FIG. 93A shows the HPLC result of Example 99. FIG. 93B shows the MALDI-TOF result of Example 99. FIG. 93C shows the CD result of Example 99.

FIG. 94A shows the HPLC result of Example 100. FIG. 94B shows the MALDI-TOF result of Example 100. FIG. 94C shows the CD result of Example 100.

FIG. 95A shows the HPLC result of Example 101. FIG. 95B shows the MALDI-TOF result of Example 101. FIG. 95C shows the CD result of Example 101. FIG. 95D shows the AFM image of the cyclic peptide complex of Example 101.

FIG. 96A shows the HPLC result of Example 102. FIG. 96B shows the MALDI-TOF result of Example 102. FIG. 96C shows the CD result of Example 102.

FIG. 97A shows the HPLC result of Example 103. FIG. 97B shows the MALDI-TOF result of Example 103. FIG. 97C shows the CD result of Example 103.

FIG. 98A shows the HPLC result of Example 106. FIG. 98B shows the MALDI-TOF result of Example 106. FIG. 98C shows the CD result of Example 106.

FIG. 99A shows the HPLC result of Example 109. FIG. 99B shows the MALDI-TOF result of Example 109. FIG. 99C shows the CD result of Example 109.

FIG. 100A shows the HPLC result of Example 110. FIG. 100B shows the MALDI-TOF result of Example 110. FIG. 100C shows the CD result of Example 110.

FIG. 101A shows the HPLC result of Example 111. FIG. 101B shows the MALDI-TOF result of Example 111. FIG. 101C shows the CD result of Example 111.

FIG. 102 shows a result of analyzing the viability of Hela cells for peptide complexes of Examples 40, 60 and 102 and nanostructures with an artificial chromosome-like structure prepared in Test Examples 13 and 14.

FIG. 103A shows the HPLC result of Example 113. FIG. 103B shows the MALDI-TOF result of Example 113.

FIG. 104A shows the HPLC result of Example 114. FIG. 104B shows the MALDI-TOF result of Example 114.

FIG. 105A shows the HPLC result of Example 115. FIG. 105B shows the MALDI-TOF result of Example 115.

FIG. 106 shows the MALDI-TOF result of Example 119.

FIG. 107 shows the MALDI-TOF result of Example 120.

FIG. 108 shows the MALDI-TOF result of Example 121.

FIG. 109A shows the HPLC result of Example 125. FIG. 109B shows the MALDI-TOF result of Example 125.

FIG. 110 shows the MALDI-TOF result of Example 128.

### DETAILED DESCRIPTION

Hereinafter, the present disclosure is described in detail.

The present disclosure is directed to providing a magnetically responsive peptide that can be applied to magnetic applications and a peptide nanostructure including the same. For this, the inventors of the present disclosure have developed a monomeric, nonpolar and perfect α-helix (MNP-helix) that has the propensity to self-assemble and align parallel to the applied magnetic field.

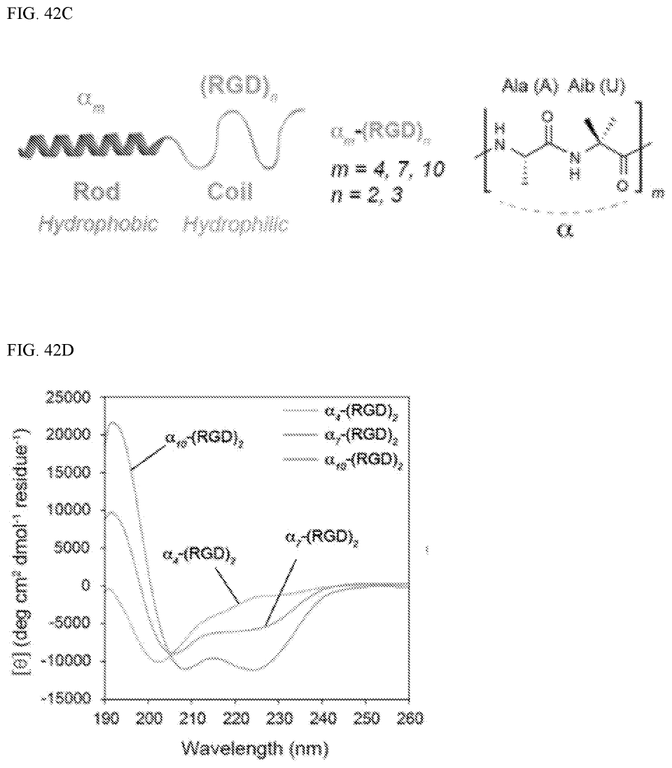

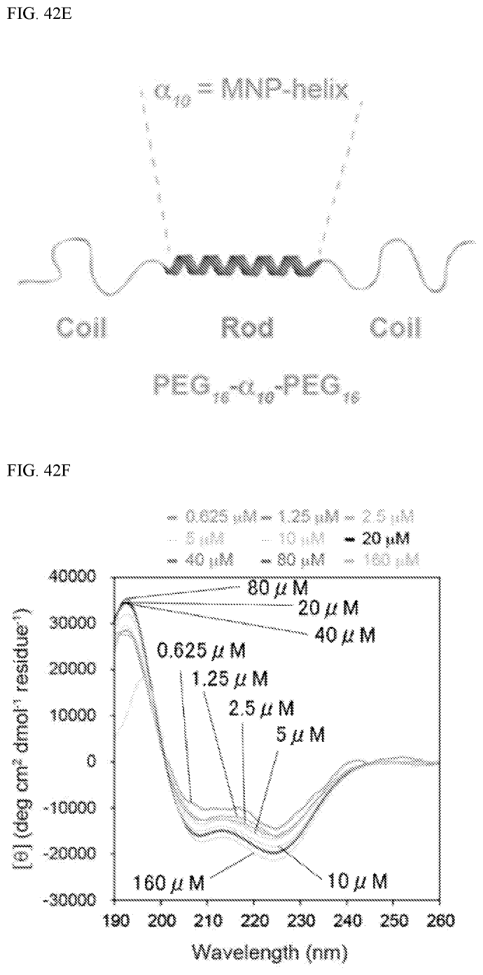

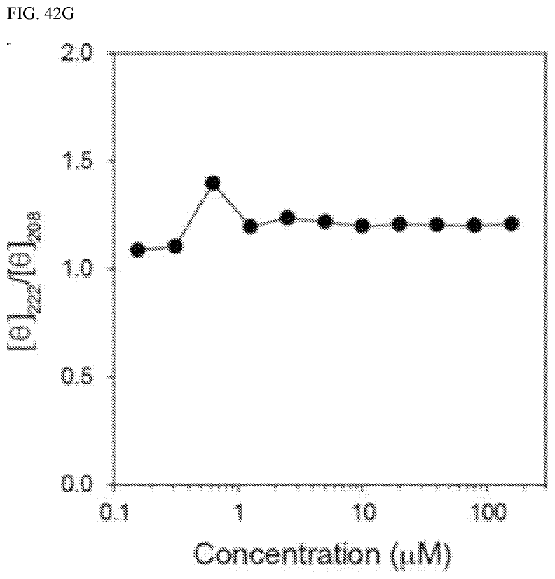

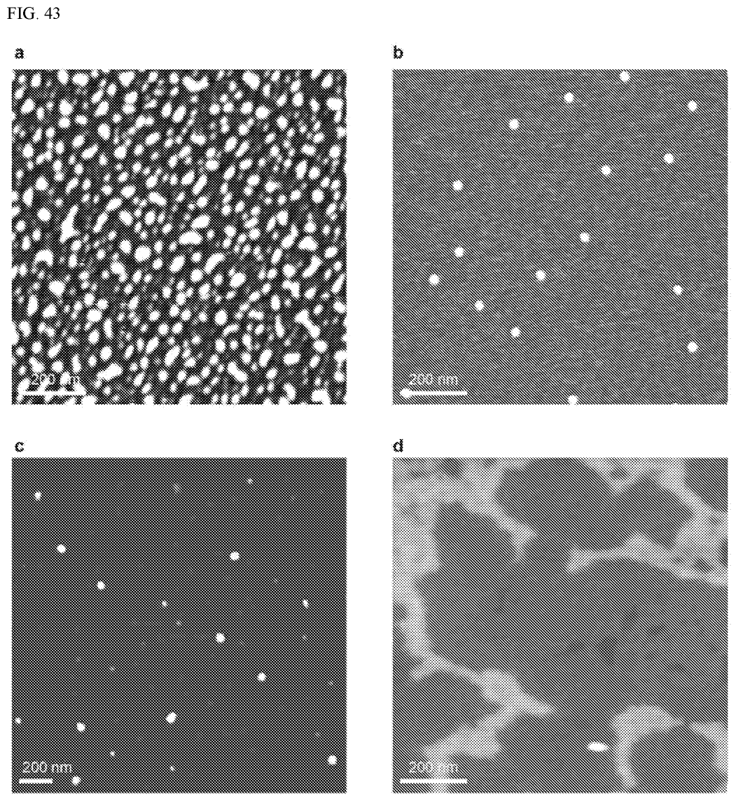

The hydrophobic peptide according to the present disclosure has an α-helix structure from among common secondary structures. Similarly to aromatic rings, the resonance-stabilized peptide bond has a meaningful value of the diamagnetic anisotropy due to it partial double bond character (FIG. 42a). In addition, because the peptide bonds are oriented parallel to the helical axis, the overall diamagnetism of the α-helix can be amplified relative to that of a single peptide bond (FIG. 42b). The hydrophobic peptide according to the present disclosure is a new structure that responds sensitively to a low magnetic field intensity because a plurality of α-helices form an aligned rod structure.

The hydrophobic peptide according to the present disclosure can form a peptide complex with a rod-coil structure by binding to a hydrophilic molecule since it acts as a supramolecular building block.

The hydrophobic peptide according to the present disclosure acts as a hydrophobic block with a rigid rod structure and forms an amphiphilic peptide complex with a rod-coil structure by binding to a hydrophilic block with a flexible coil structure.

The peptide complex having a rod-coil structure has more advantages over the existing molecules with a coil-coil structure in terms of well-defined, dynamic and responsive assembly because the hydrophobic block consisting of hydrophobic peptides imparts orientational organization and the entropic penalty associated with chain stretching in coil-coils can be reduced.

The existing peptides having an α-helix rod structure cannot maintain the α-helix structure in the monomeric state. Also, in proteins, most α-helix peptide sequences can be stabilized only when they make noncovalent interactions with other residues of the proteins. Single α-helix structures found in proteins and some of designed peptides are highly helical in the monomeric state. These helix structures cannot be used as supramolecular building blocks because all of them are highly charged and hydrophilic.

In order to solve the problems described above, the inventors of the present disclosure have explored the synergistic effect of the α-helix and rod-coil structures to develop organic molecules responsive to the magnetic field produced by permanent magnets. To this end, they have designed a new hydrophobic peptide having an MNP α-helix structure as a first step.

By using the hydrophobic peptide according to the present disclosure as a rod block of a rod-coil peptide complex having amphiphilicity, they have confirmed that the self-assembly and disassembly processes can be controlled under a relatively weak magnetic field (0.07-0.25 T).

In addition, since the peptide complex including the hydrophobic peptide of the present disclosure is positively charged, it interacts with negatively charged materials. Especially, it self-assembles with genetic materials (nucleic acid, etc.) to form a nanostructure with an artificial chromosome-like structure. The nanostructure is a new-concept magnetically responsive organic molecule that can safely store and deliver a genetic material by packaging tightly and the release of the genetic material can be controlled with a magnetic field. It was found out that the nanostructure can be usefully used in various fields of applied researches such as medicine, cosmetics, stimuli-responsive molecular machines, motion control of organic materials, etc. because its magnetic responsiveness can be controlled variously.

In an aspect, the present disclosure relates to a hydrophobic peptide represented by General Formula 1 or 2.

-{[Aib]-[Xaa1]}ₘ- [General Formula 1]

-[Xaa2]n-{[Aib]-[Xaa1]}m- [General Formula 2]
In the formulas,
each of Xaa1 and Xaa2 is independently a D- or L-amino acid residue selected from a group consisting of Ala (A), Ile (I), Leu (L), Met (M) and Val (V)
Xaa1 is a D- or L-amino acid residue selected from a group consisting of Ala (A), Ile (I), Leu (L), Met (M) and Val (V),
n is the integer 1, and
m is any integer selected from 4 to 50.

In the present specification, the term “peptide” or “polypeptide” refers to a linear molecule formed as 4-100, specifically 4-80, more specifically 10-80, more specifically 20-80, most specifically 20-40, amino acid residues are linked by peptide bonds.

The hydrophobic peptide represented by General Formula 1 or 2 may be represented by SEQ ID NO 121 or 122. It is not derived from existing proteins but was designed to include [Aib]-[Xaa1] or [Xaa1]-[Aib] as a repeat unit (hereinafter, also referred to as ‘a’) through numerous combinations of existing amino acid residues so as to stably maintain the α-helix structure and impart hydrophobicity and a rod structure.

Through experiments, the inventors of the present disclosure have identified that the hydrophobic peptide has a stable α-helix structure and rod structure despite the short sequence length. In addition, they have identified that it can be aligned in real time in response to a weak magnetic field and forms an artificial chromosome-like structure by interacting with a genetic material.

In the present disclosure, the hydrophobic peptide of General Formula 1 or 2 is a newly designed sequence derived from nowhere. Among the existing peptide secondary structures, the α-helix structure is known to have increased stability as the length of the peptide chain increases. However, as the peptide length increases, it is difficult to synthesize the peptide and to prepare a supramolecular building block having a specific structure. As a result of actually preparing peptides consisting of short repeat units through various combinations of amino acid residues, it was confirmed that most of them failed to form α-helix structures or, even if they did, the structures existed in unfolded states.

Through the experiments described above, the inventors of the present disclosure have identified that a peptide having a repeat unit prepared from a hydrophobic amino acid residue such as alanine (Ala or A) and Aib (2-aminoisobutyric acid, U) has a stable α-helix structure while having appropriate hydrophobicity for self-assembly and can be aligned highly by a magnetic field.

In a specific exemplary embodiment of the present disclosure, the hydrophobic peptide of the present disclosure is advantageous in that it has a perfectly stabilized α-helix structure while existing as monomeric state in solution without aggregation.

In a specific exemplary embodiment of the present disclosure, m may be any integer selected from 4 to 50, more specifically any integer selected from 6 to 13, further more specifically any integer selected from 6 to 10, most specifically 10.

In a specific exemplary embodiment of the present disclosure, Xaa1 may be any one selected from a group consisting of Ala (A), Ile (I), Leu (L) and Met (M), specifically Ala (A) or Leu (L).

In the formulas, Xaa1 may be most specifically alanine (Ala or A) so that the peptide has a stable α-helix structure while having appropriate hydrophobicity for self-assembly and can be aligned highly by a magnetic field.

In addition, in a specific exemplary embodiment of the present disclosure, Xaa1 may be an L-amino acid residue or a D-amino acid residue, although not being specially limited thereto.

In a specific exemplary embodiment of the present disclosure, the hydrophobic peptide represented by General Formula 1 or General Formula 2 is a polypeptide consisting of a repeat unit with the shortest sequence having a newly designed α-helix structure. The hydrophobic peptide may be any one selected from SEQ ID NOS 1-36, specifically any one selected from SEQ ID NOS 5-19 and 24-36, more specifically any one selected from SEQ ID NOS 13-14 and 32-33.

The hydrophobic peptide has a stable α-helix secondary structure while exhibiting hydrophobicity, is aligned along the direction of an external magnetic field and has superior responsiveness to a weak magnetic field of 1 T or lower.

Due to the characteristics described above, the hydrophobic peptide according to the present disclosure has a rigid rod structure capable of stably maintaining a plurality of α-helix structures.

The arrangement and orientation of the hydrophobic peptide according to the present disclosure are controlled under a magnetic field. As described above, the peptide is aligned and oriented parallel to the direction of an external magnetic field.

The existing peptide cannot maintain its α-helix structure unless it exists within a specific structure and cannot respond to a magnetic field because it is a very weak diamagnetic material. In contrast, the peptide of the present disclosure, which consists of a repeat unit with the shortest sequence, [Aib]-[Xaa1] or [Xaa1]-[Aib] (hereinafter, also referred to as ‘a’), maintains a stable α-helix structure in monomeric state even when it is not constrained in a ring or a large protein. In addition, it is a new magnetically responsive peptide that is aligned and oriented parallel to the direction of an external magnetic field.

Furthermore, since the hydrophobic peptide according to the present disclosure is easy to impart new functions through binding with a hydrophilic polymer or a hydrophilic peptide without losing the above-described characteristics and can be stored and delivered in the form of micelle particles through self-assembly, it can be usefully used in various fields such as medicine, stimuli-responsive molecular machines, cosmetics, etc.

In addition, since the hydrophobic peptide according to the present disclosure does not affect the genetic information analysis method such as PCR, even when mixed with a nucleic acid material, without generating noises, it can be advantageously used for analysis of a nucleic acid material without an additional purification process.

In another aspect, the present disclosure provides a peptide complex: including the hydrophobic peptide; and at least one hydrophilic polymer or hydrophilic peptide bound to one or both ends of the hydrophobic peptide.

As described above, in the peptide complex according to the present disclosure, one or more hydrophilic polymer or hydrophilic peptide may be bound to one or both ends of the hydrophobic peptide.

The description of the hydrophobic peptide will be omitted to avoid redundancy.

The one end of the hydrophobic peptide refers to any of the N-terminal or C-terminal of the hydrophobic peptide, and the both ends refers to both the N-terminal and C-terminal of the hydrophobic peptide.

The ‘N-terminal’ refers to the first amino acid residue in the hydrophobic peptide sequence. The N-terminal residue contains a free α-amino group. The ‘C-terminal’ refers to the last amino acid residue in the hydrophobic peptide sequence. The C-terminal residue contains a free carboxylate group.

In the present disclosure, the ‘polymer’ refers to a macromolecule having repeat units linked by covalent bonds. The polymer may be hydrophilic, hydrophobic or amphiphilic. Specifically, it may be a hydrophilic polymer exhibiting hydrophilicity. The polymer may include a homopolymer, a random copolymer and a block copolymer.

The ‘hydrophilic polymer’ is not specially limited as long as it substantially miscible with water. It may be specifically polyethylene glycol (PEG), poly(N-(2-hydroxypropyl)methacrylamide), poly(2-(methacryloyloxy)ethyl phosphorylcholine), poly(hydroxyalkyl L-asparagine) or poly(hydroxyalkyl L-glutamine), more specifically polyethylene glycol (PEG).

Specifically, the hydrophilic polymer may be an unbranched, non-crosslinked linear polymer and may have a molecular weight of about 1-100 kDa, specifically about 1-75 kDa, more specifically about 5-50 kDa, further more specifically about 5-25 kDa.

The polyethylene glycol (PEG) contains PEG monomers and may be represented by the chemical formula CH₃—O(CH₂CH₂O)ₙ— or —O(CH₂CH₂O)ₙ—, wherein n is a positive integer from about 10 to about 2,300.

The ‘hydrophilic peptide’ is not specially limited as long as it is a peptide exhibiting hydrophilicity. As known to those skilled in the art, it refers to a peptide exhibiting hydrophilicity as a whole with a high proportion of polar amino acid or hydrophilic amino acid residues. The hydrophilic amino acids may include H (histidine), N (asparagine), Q (glutamine), S (serine), T (threonine), C (cysteine), G (glycine), K (lysine), R (arginine), E (glutamic acid) and D (aspartic acid).

The hydrophilic peptide may consist of 3-100, specifically 3-60, more specifically 3-20, amino acid sequences and the content of the hydrophilic amino acids may be 70-100%.

The hydrophilic peptide is not specially limited as long as the above-described conditions are satisfied. Specifically, it may be an optional ligand that may or may not be present, selected from RGD, transferrin, folate, a signal peptide or signal sequence, a localization signal or sequence, a nuclear localization signal or sequence (NLS), an antibody, a cell-penetrating peptide (e.g., TAT or KALA), a ligand of a receptor, a cytokine, a hormone, a growth factor, a small molecule, a carbohydrate such as mannose and galactose, a synthetic ligand, a small-molecule agonist and an inhibitor or antagonist of a receptor (e.g., an RGD peptidomimetic analogue).

Specifically, the hydrophilic peptide may include or consist of the following amino acid sequences.

### Hydrophilic Peptide

TABLE 1

| SEQ ID NO | Sequence |
|---|---|
| 98 | RGDRGD |
| 99 | RGDRGDRGD |
| 100 | RGDRGDRGDRGD |
| 101 | GSG |
| 102 | GSGSG |
| 103 | GSGSGSG |
| 104 | GSGSGSGSG |
| 105 | GHHHHHHHHHHHGRGDRGDRGDG |

The hydrophobic peptide and the hydrophilic polymer or hydrophilic peptide are linked by a chemical bond. They may be linked either directly or indirectly via a linker.

The linkage via a chemical bond or a linker may vary depending on the terminal sequence of the peptide and the terminal group of the polymer. It may be for example, an amide bond, an ether bond, a thioether bond, an ester bond, a thioester bond, a carbonate bond, a carbamate bond, a phosphate bond or an oxime bond. Specifically, it may be an amide bond.

The linker may be any one that can be linked to the terminal of the hydrophobic peptide, the terminal of the hydrophilic peptide or the terminal of the hydrophilic polymer and can form the chemical bonds described above.

For example, dicyclohexylcarbodimide (DCC), 1,4-bis-maleimidobutane (BMB), 1,11-bismaleimidotetraethylene glycol (BM(PEG)₄), 11-ethyl-3-[3-dimethylaminopropyl]carbodiimide hydrochloride (EDC), succinimidyl-4-[N-maleimidomethylcyclohexane-1-carboxy-[6-amidocaproate] (SMCC) and a sulfonate thereof (sulfo-SMCC), succinimidyl 6-[3-(2-pyridyldithio)-ropionamido]hexanoate (SPDP) and a sulfonate thereof (sulfo-SPDP), m-maleimidobenzoyl-N-hydroxysuccinimide ester (MBS) and a sulfonate thereof (sulfo-MBS), succinimidyl[4-(p-maleimidophenyl) butyrate (SMPB) and a sulfonate thereof (sulfo-SMPB), etc., specifically dicyclohexylcarbodimide (DCC), may be used. A linker peptide consisting of 1-10 amino acids may also be used.

The linker peptide may consist of 1-10 amino acids selected from H (histidine), G (glycine) and R (arginine).

The peptide sequence may include silent mutations that may occur during an encoding process. The silent mutations may be intended implicitly. The peptide sequence of the present disclosure may include sequence variations for conservative substitutions that provide functionally similar amino acids widely known in the art.

The peptide complex of the present disclosure may further contain a positively charged peptide consisting of positively charged 1-4 amino acid residues at the N- or C-terminal.

The positively charged peptide may be linked to the hydrophobic peptide of the peptide complex of the present disclosure. Specifically, the positively charged peptide is linked via a linker or a direct bond. The design is intended to provide a new approach to easily form a bond with a negatively charged molecule such as an oligonucleotide for better efficiency such as delivery of a nucleic acid material, treatment, storage, etc.

The peptide complex of the present disclosure may form a nanostructure by binding easily to a nucleic acid material even without a positively charged peptide bound thereto. However, when it contains a positively charged peptide, a nanostructure with a highly functional structure in artificial chromosome form may be prepared more effectively.

The positively charged peptide may be any one selected from SEQ ID NOS 108-119.

The peptide complex of the present disclosure may be an amphiphilic molecule having a rod-coil structure. Since the peptide complex of the present disclosure has amphiphilic characteristics, it can form a self-assembled structure in an aqueous solvent through self-assembly.

The peptide complex of the present disclosure may self-assemble into a micelle structure in an aqueous solvent through self-assembly. The micelle structure refers to an aggregated nanostructure of amphiphilic molecules with regular size and shape. The micelle has various shapes including spherical, ellipsoid, cylindrical, ring and lamellar shapes. The peptide complex of the present disclosure may self-assemble to one or more shapes selected from spherical, ellipsoid, cylindrical, ring and lamellar shapes.

The self-assembled structure of the peptide complex of the present disclosure may have an average diameter of usually 10-400 nm.

The form of the self-assembled structure may be controlled by controlling the orientation of the peptide complex according to the present disclosure with a magnetic field. When an electric field is not applied, the peptide complex of the present disclosure is prepared into a spherical nanoparticle as the orientation of the α-helix structure disappears. When an electric field is applied, a self-assembled structure having an anisotropic planar nanostructure or cruciform anisotropic nanostructure may be prepared as the α-helix structure is oriented along the direction of the electric field.

The peptide complex according to the present disclosure responds sensitively to a very low magnetic field intensity of 0.1-2 T.

In another aspect, the present disclosure relates to a composition for storing nucleic acid information safely, which contains the peptide complex and a nucleic acid material.

The peptide complex and the nucleic acid material may self-assemble into a nanoribbon or a nanostructure having an artificial chromosome-like structure via noncovalent bonding.

The composition of the present disclosure can store and deliver the genetic material very stably since the nucleic acid material is surrounded and coated by the peptide complex through interaction between the peptide complex and the nucleic acid material. The above-described process is advantageous in that it proceeds easily and quickly because the self-assembly occurs automatically in the aqueous solvent. In addition, the composition of the present disclosure is advantageous in that the information of the genetic material can be confirmed by usefully used DNA analysis without an additional separation process because the peptide complex does not affect the analysis process such as PCR, etc.

A nanoribbon structure may be formed through self-assembly as the peptide complex binds to the nucleic acid material and a nanostructure with an artificial chromosome-like structure may be formed through self-assembly as the nanoribbon is folded and stacked.

In the present disclosure, the artificial chromosome-like structure refers to a structure formed by condensation of chromatin, a thread-like structure made of nucleic acids and proteins that carries the genetic information of living organisms such as human chromosomes. In the present disclosure, the nanoribbon is formed as the peptide binds to the nucleic acid material and the nanoribbon is condensed to form a chromosome-like system that stores a large amount of genetic information in the minimal space.

The nucleic acid material may be one or more selected from a group consisting of an RNA, a DNA, an siRNA (short interfering RNA), an aptamer, an antisense ODN (oligodeoxynucleotide), an antisense RNA, a ribozyme and a DNAzyme. For example, it may be an RNA, a DNA or a cDNA, and the sequence of the nucleic acid may be a coding region sequence or a non-coding region sequence (e.g., an antisense oligonucleotide or siRNA). As the nucleic acid cargo, a nucleotide may be a standard nucleotide (e.g., adenosine, cytosine, guanine, thymine, inosine or uracil) or an analog thereof (e.g., a phosphorothioate nucleotide). For example, the nucleic acid cargo may be an antisense sequence consisting of phosphorothioate nucleotides or RNAi.

Since the structural degradation of the nanostructure can be induced under a magnetic field intensity of 0.1-2 T, the material release of the nucleic acid can be induced at desired time depending on purposes. Using these properties, it is possible to implement a delivery system which allows the control of long-term storage and release of the nucleic acid material.

Since the composition according to the present disclosure requires 10 days or longer for complete degradation under static magnetic field intensity, the rotating magnetic field condition is preferred for degradation within 1 day. The intensity of the rotating magnetic field may be specifically 0.1-0.5 T, although it can be adjusted adequately depending on the time required for the degradation and purpose.

In a specific exemplary embodiment of the present disclosure, the composition according to the present disclosure is very useful as a system for storing and protecting the information of a nucleic acid material for a long time since it perfectly protects the nucleic acid material from external environment and can restore the genetic information at any time through analysis methods such as PCR.

The composition according to the present disclosure is advantageous in that the genetic information of the nucleic acid material can be confirmed directly through analysis methods such as PCR without an additional purification process.

Hereinafter, the present disclosure will be described in more detail through specific examples. However, these examples are only for describing the present disclosure more specifically and it will be obvious to those having ordinary knowledge in the art that the scope of the present disclosure is not limited by them.

### Experimental Methods

### CD (Circular Dichroism) Spectroscopy

To evaluate the structures of a linear plasmid DNA and peptides, CD spectra were analyzed using a Chirascan CD spectrometer (Applied Photophysics, UK). The concentration of the peptides typically ranged from 0.156 to 160 μM. The final concentration of the linear plasmid DNA was 30 ng/μL. All the samples were dissolved in H₂O. For the formation of the artificial chromosome, a mixture of the peptide and the DNA was incubated for 12 days or longer. Scanning was performed using a 2-mm path-length cuvette. Each scan was repeated three times and averaged. MRE (mean residue ellipticity) was calculated based on the number of amino acid residues.

MCD (magnetic circular dichroism) spectra were obtained using a Jasco-815 159-L spectrophotometer (Jasco, Japan). The concentration of the peptides was 20 μM in distilled water. Measurements were performed using a 2-mm path-length cuvette. The magnetic field of the permanent magnet in the MCD accessory (PM-491) was 1.6 T. Each scan was repeated three times and averaged.

### NMR (Nuclear Magnetic Resonance) Spectroscopy

¹H NMR and ²D ¹H-¹⁵N IPAP-HSQC (in-phase/anti-phase heteronuclear single quantum coherence) NMR spectra were acquired at 298 K using a Bruker AVANCE IV 900 MHz spectrometer and a BrukerAVANCE Ill HD 700 MHz spectrometer, both equipped with cryogenic probes. The NMR sample of PEG_{30-α10-}PEG₃₀ was dissolved in methanol-d₃/chloroform-d (1:1) containing 0.05% TFA at a concentration of 4.26 mM. The acquisition parameters of IPAP were 2048 t₂×1024 t₁ points, 8 scans, 0.2 second recycle delay, 14 ppm spectral width for ¹H and 26 ppm spectral width for ¹⁵N. The acquired data were split into two data sets, IP and AP, using a TopSpin software (Bruker, Germany). Each spectrum was processed by zero-filling up to 8192×8192 points. Peak positions were assigned using an NMRFAM-SPARKY software.

### AFM (Atomic Force Microscopy) Analysis

AFM was performed using an NX10 system (Park Systems, Korea) in a non-contact mode with PPP-NCHR AFM probes (Nanosensors, Switzerland). The DNA concentration ranged from 1 to 30 ng/μL and the peptide concentration ranged from 1 to 32 μM (in H₂O). 2 μL of the sample solution was cast onto a freshly cleaved mica surface and dried. The data obtained by the SmartScan program (Park Systems, Korea) was analyzed using the XEI program (Park Systems, Korea).

### TEM (Transmission Electron Microscopy) and Electron Diffraction

TEM was performed using a JEM-F200 multi-purpose electron microscope (JEOL, Japan) at 200 kV. 2 μL of the sample solution was loaded on a copper grid (carbon type-B grid or Formvar/silicon monoxide grid, 200 mesh copper grids with a 97 μm hole; Ted Pella, USA). 1 hour later, 2 μL of 1-2% uranyl acetate was added to the dried sample for less than 1 minute and the negative stain solution was wicked off with filter paper. To analyze the internal packing structure of the artificial chromosome, the electron diffraction (ED) pattern was observed in selected areas.

### EMSA (Electrophoretic Mobility Shift Assay)

EMSA was performed to investigate the mode of interaction between the peptide complex and the linear plasmid DNA and their binding ratio. The concentration of the peptide complex was increased from 1.5 μM to 6214 μM, while the concentration of the linear plasmid DNA was fixed to 30 ng/μL. The same mixing protocol was used as in the artificial chromosome formation. After incubation at room temperature, 10% glycerol was added to the sample for gel loading. Electrophoresis was performed for 100 minutes at 90 V on 1% agarose gel. The bands were visualized by staining with an SYBR Safe DNA gel stain (Invitrogen, USA).

### DNase I Protection Assay

A sample (the linear plasmid DNA or the artificial chromosome) containing 60 ng (2 μL; 30 ng/μL) of DNA was mixed with 0.1 μL (0.2 unit) of DNase I and 1 μL of a 10× DNase I reaction buffer. The volume of the mixture was 10 μL. After 20 minutes of incubation at 37° C., 0.3 μL of 0.05 M EDTA was added to inactivate the enzyme. Then, the sample was mixed with 1.7 μL of 60% glycerol and electrophoresed on 1.1% agarose gel (120 minutes, 100 V). For visualization, the DNA was stained with an SYBR Safe DNA gel stain (Invitrogen, USA). To evaluate the time-dependent kinetics of DNA degradation, UV absorbance at 260 nm was recorded over time after the addition of DNase I. Each sample (71.2 μL) containing DNA (30 ng/μL) and a 10× DNase I reaction buffer (8 μL) was mixed in a cuvette. Then, DNase I (0.8 μL) was added to the cuvette and the sample was mixed by pipetting for about 90-105 seconds. UV absorbance at 260 nm (A₂₆₀) was measured using a V-650 UV-vis spectrophotometer (JASCO, Japan). A quartz cuvette with a path length of 10 mm was used. The absorbance was recorded at intervals of 10 seconds.

### Information Recovery from Artificial Chromosome

To assess the capability of recovering DNA information stored in the artificial chromosome, PCR (polymerase chain reaction) was conducted to amplify a specific DNA fragment of the sample DNA. The specific 310-bp fragment was amplified using the sense primer 5′-ACG GAG ACT GGA GTC GAA GAG G-3′ (SEQ ID NO 106) and the antisense primer 5′-GTA GGG CAA CTA GTG CAT CTC CC-3′ (SEQ ID NO 107). Each reaction mixture (50 μL) contained 50 ng of DNA, 10 pmol of each primer and 25 μL of 2× Quick Taq HS DyeMix (Toyobo, Japan). PCR amplification (30 cycle) was performed using a T100 thermal cycler (Bio-Rad, USA). After the amplification, the product was electrophoresed for 120 minutes at 80 V on 2% agarose gel (TBE system). A SYBR Safe DNA gel stain (Invitrogen, USA) was used for DNA staining.

### EXAMPLES AND TEST EXAMPLES

### Examples 1-36. Synthesis of Hydrophobic α-Helix Peptides (α₄₋₁₃)

Hydrophobic α-helix peptides (α₄₋₁₃) represented by SEQ ID NOS 1-36 were synthesized as follows. The peptides were synthesized by standard Fmoc solid-phase peptide synthesis (SPPS) on Rink Amide MBHA resin LL (100-200 mesh, 0.30-0.40 mmol g⁻¹, Novabiochem, Germany). All Fmoc-amino acids, Fmoc-NH-PEG8-propionic acids and Fmoc-NH-PEG10-propionic acids were purchased from AAPPTec (USA).

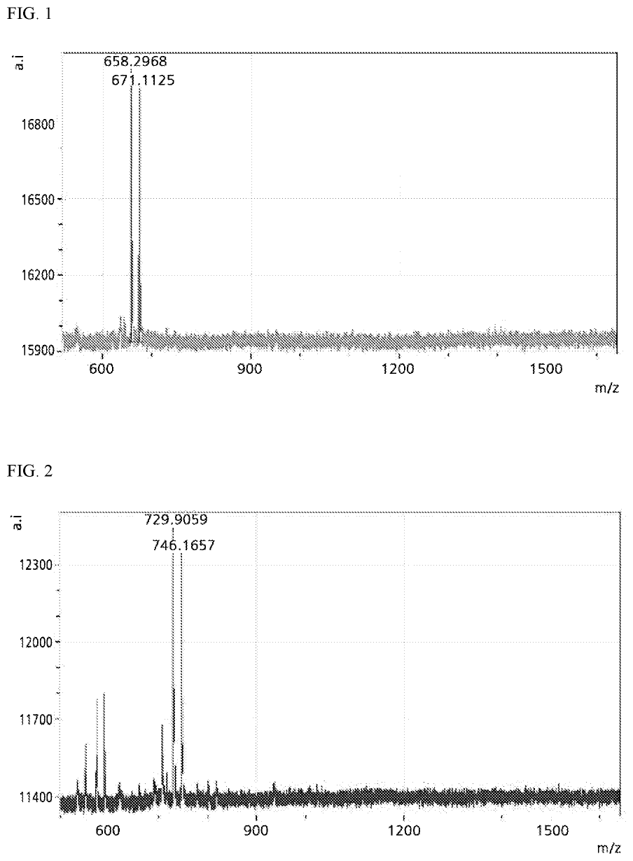

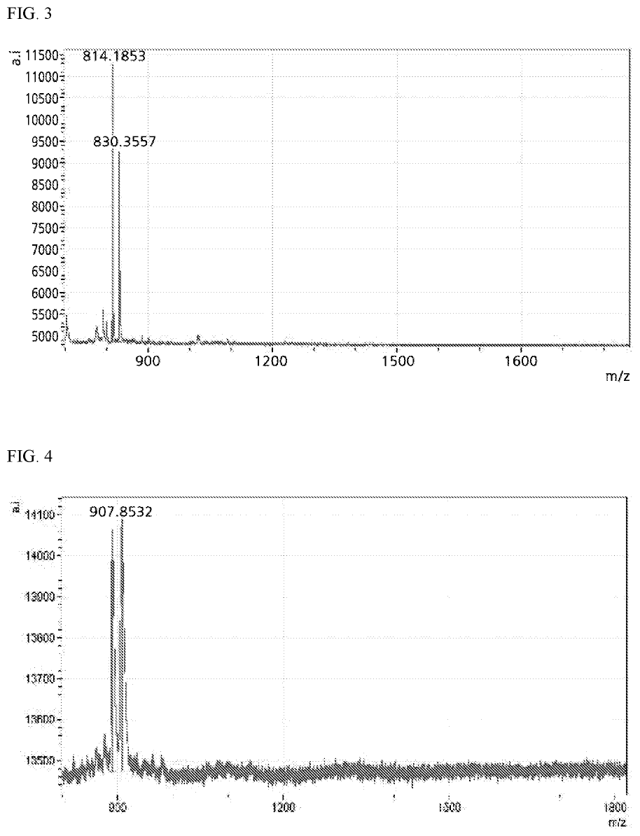

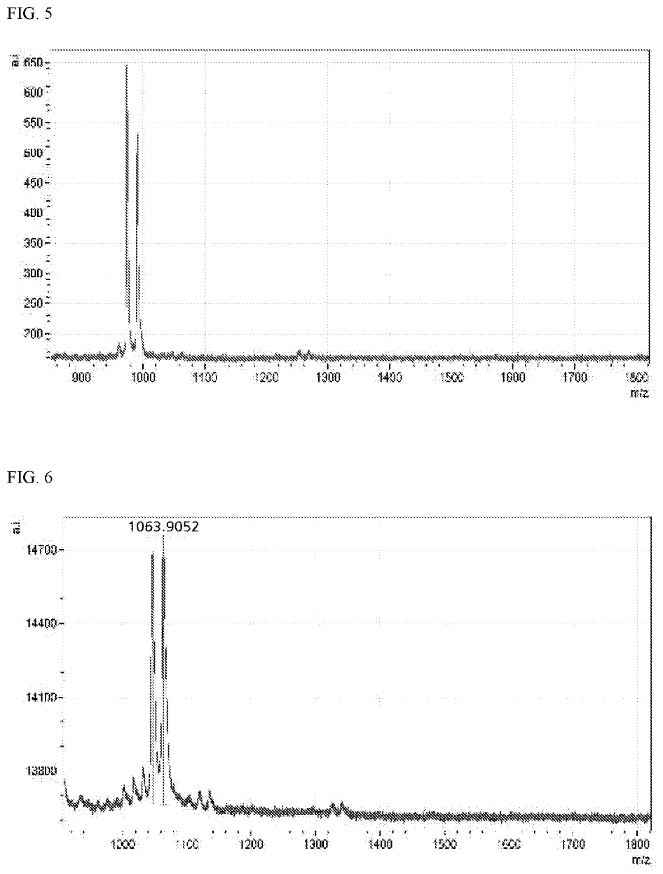

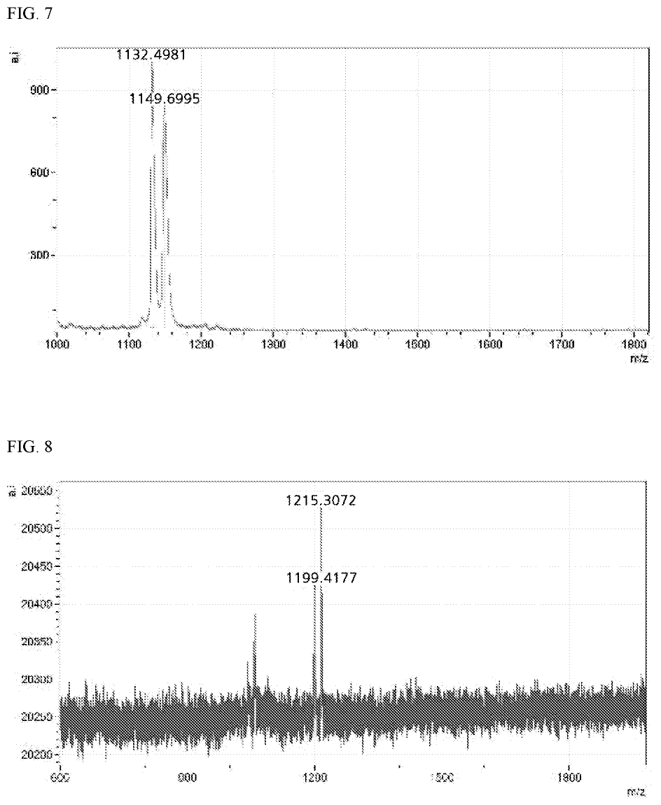

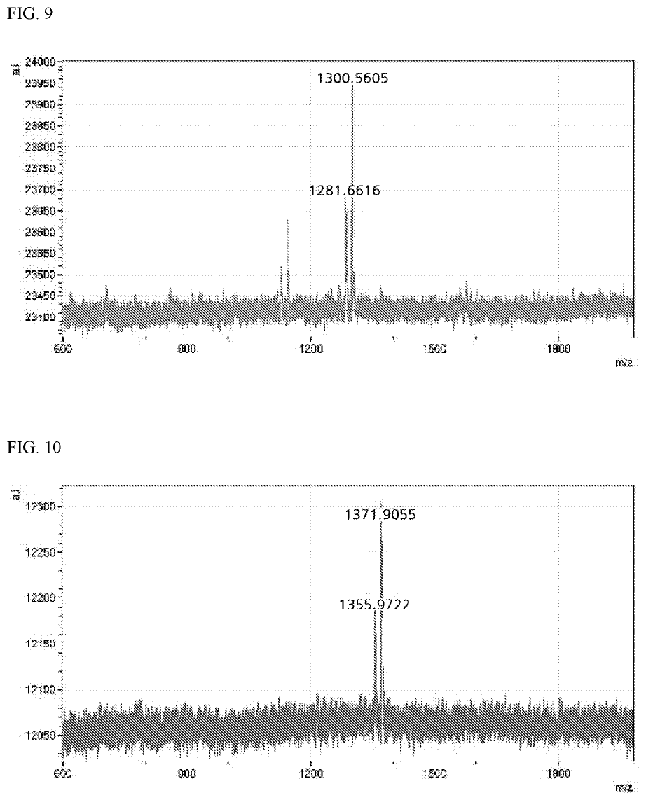

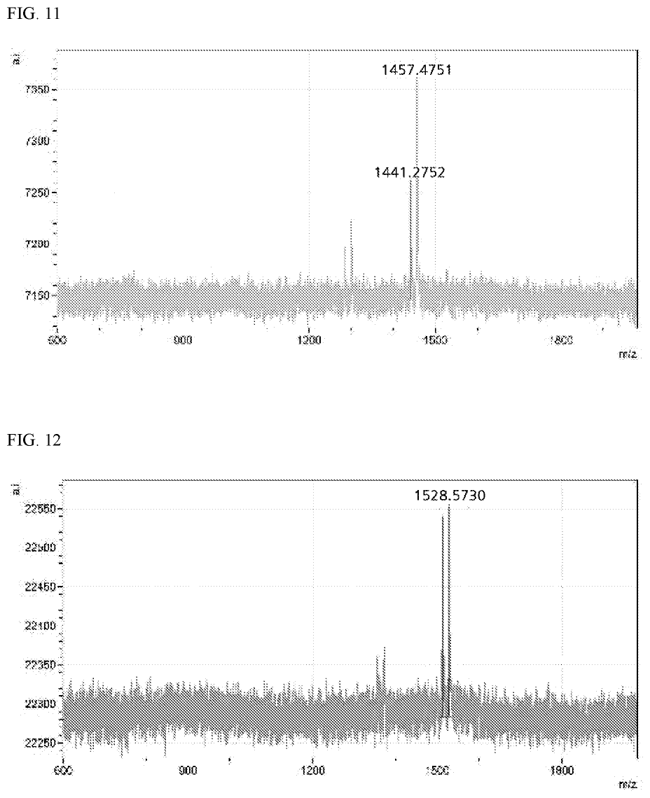

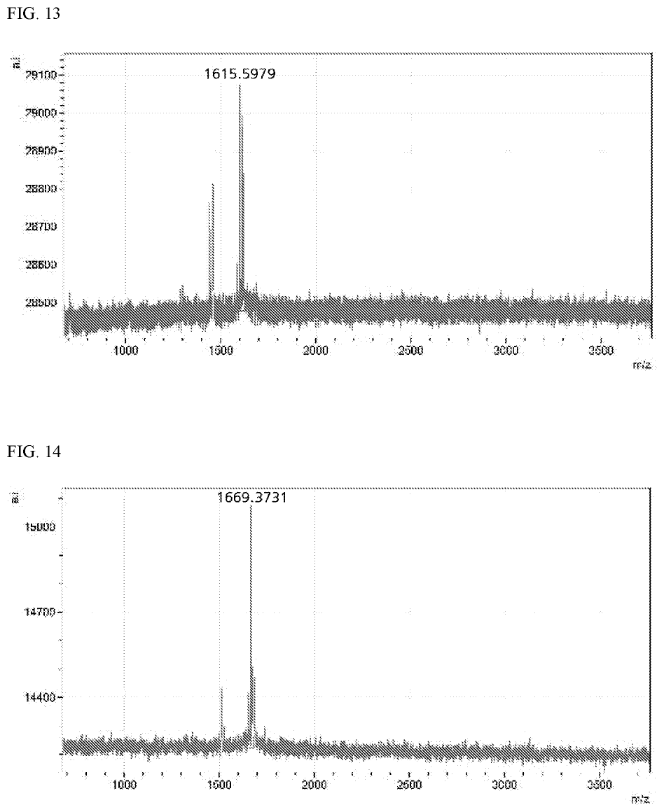

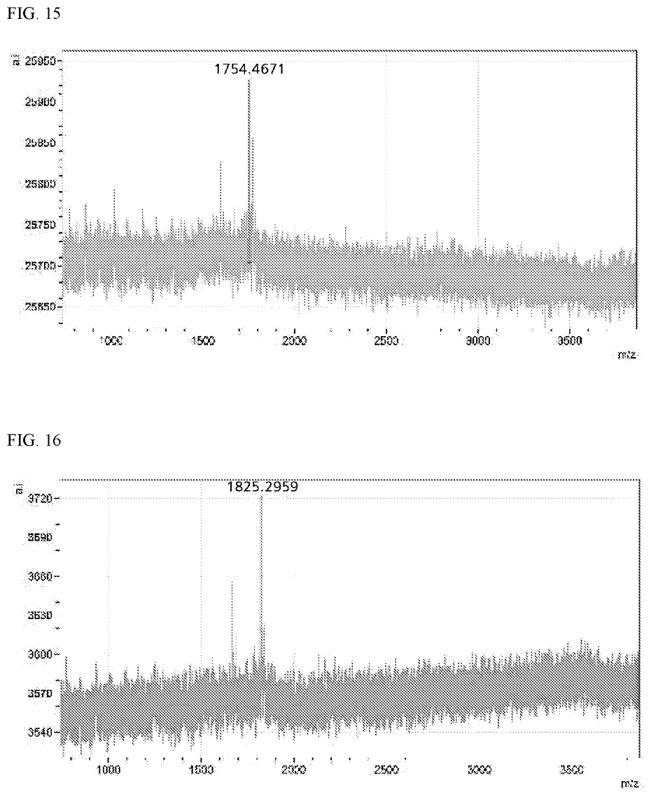

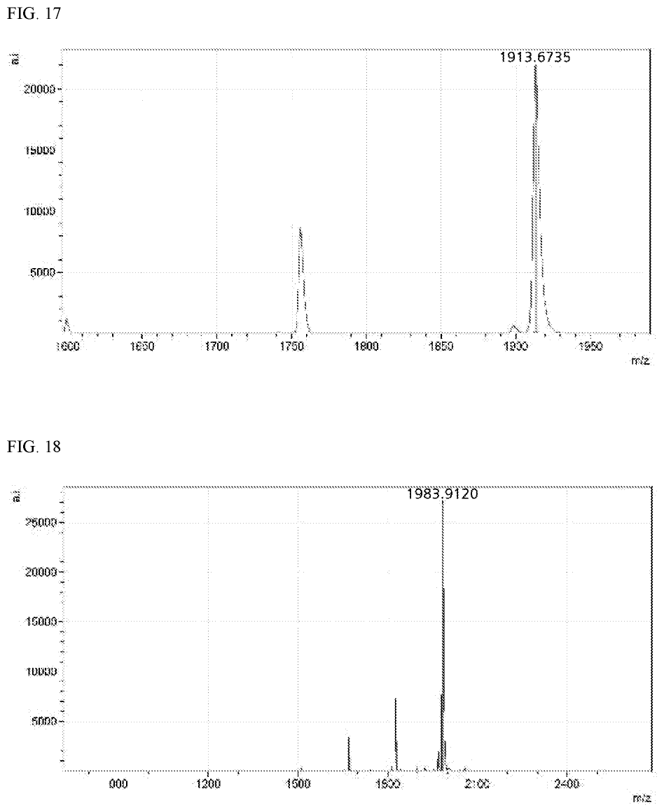

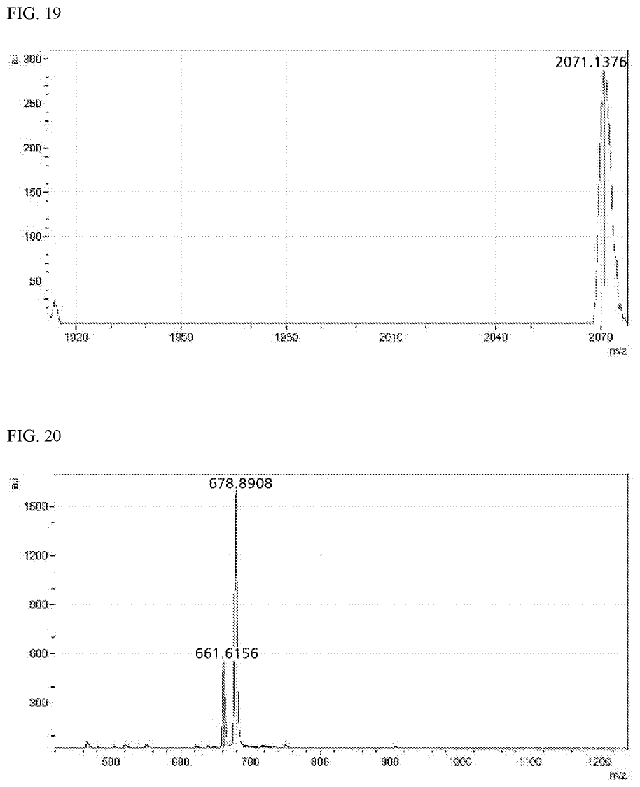

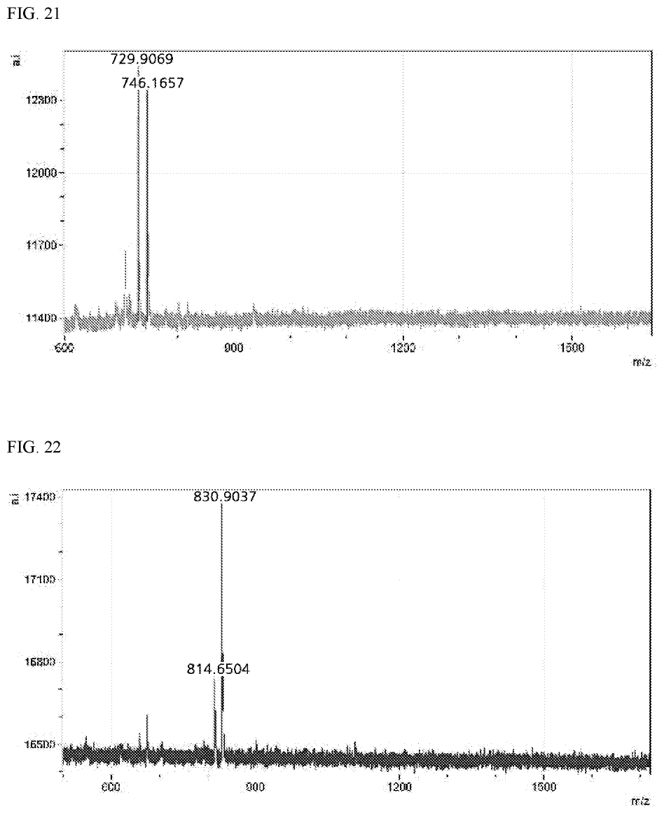

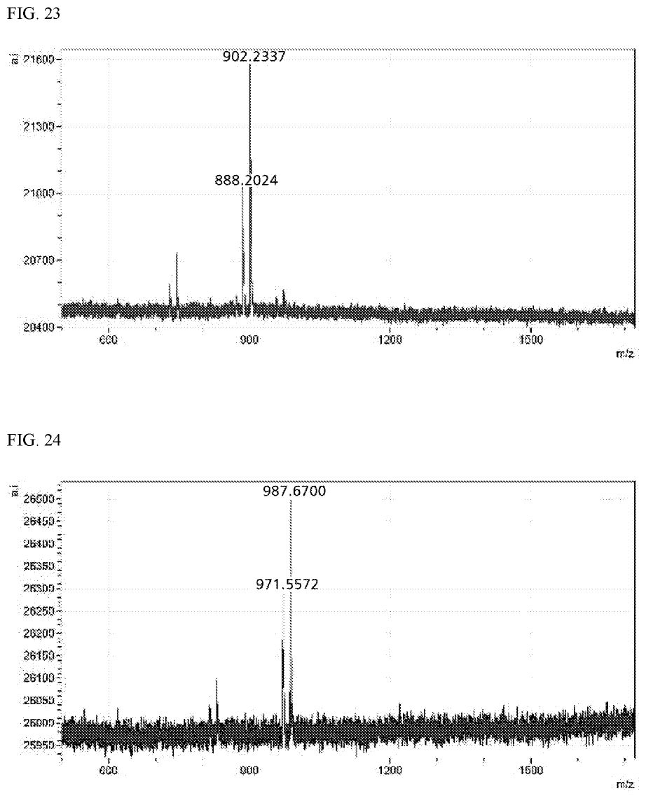

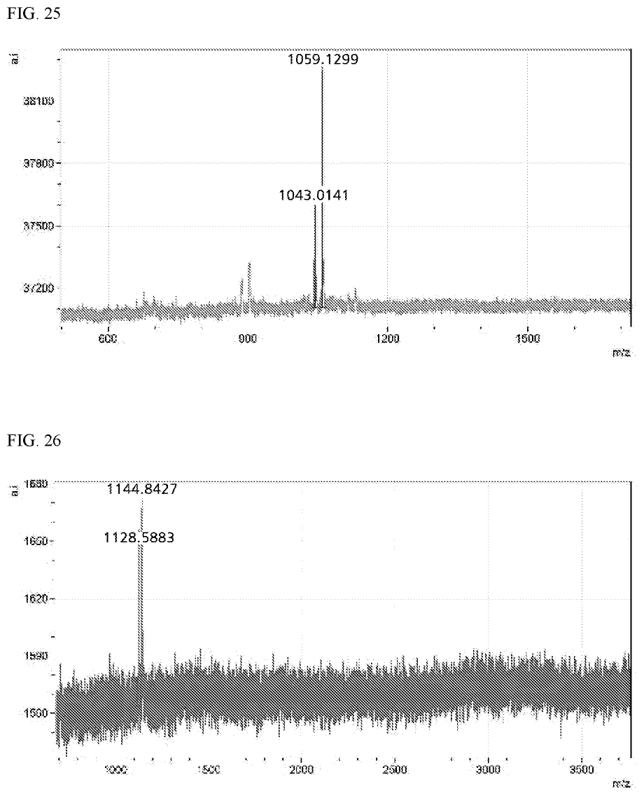

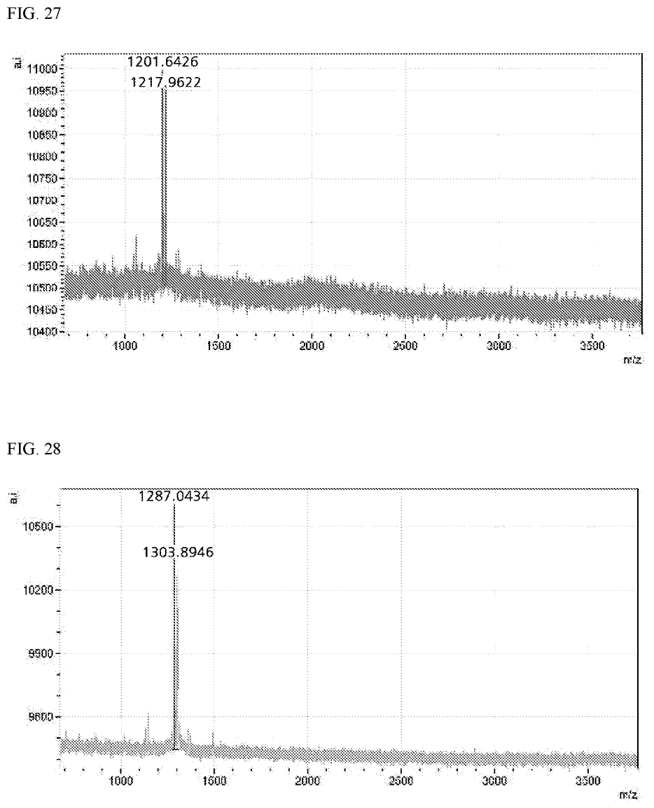

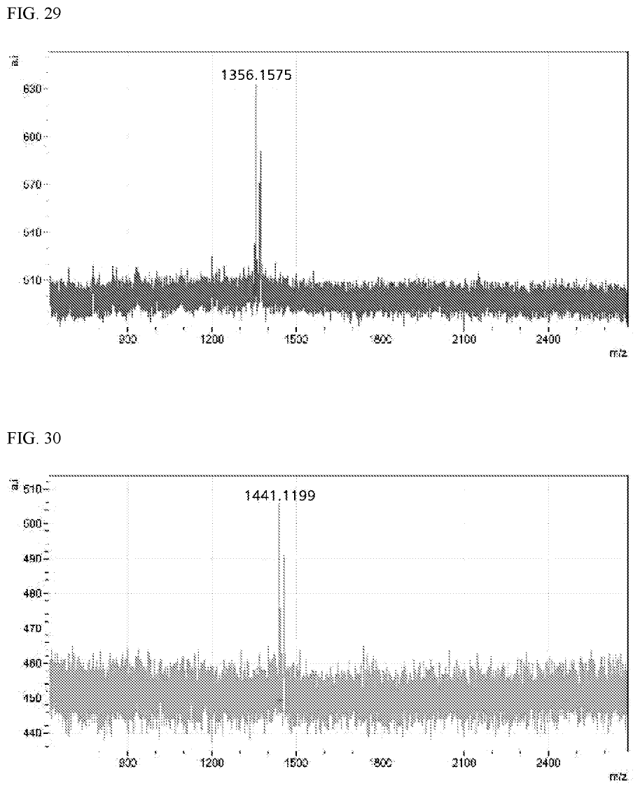

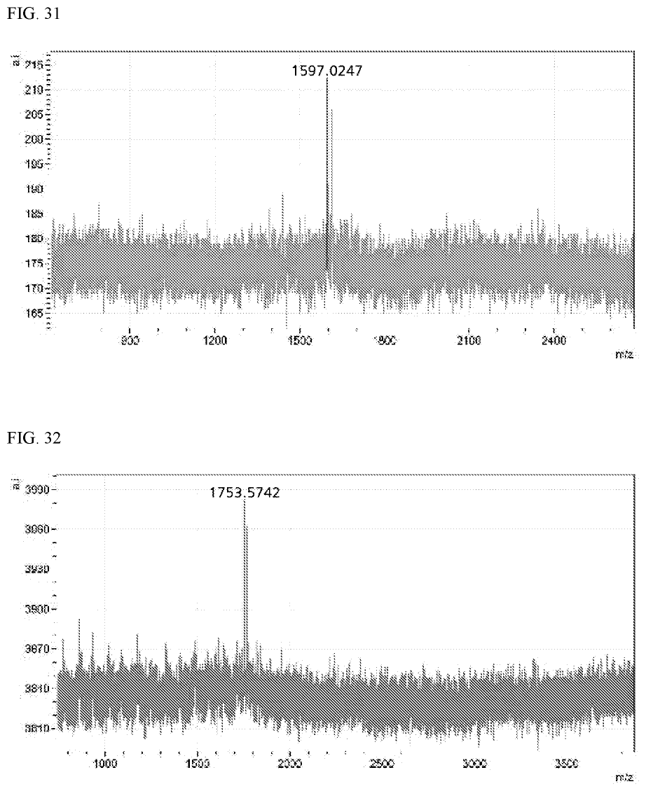

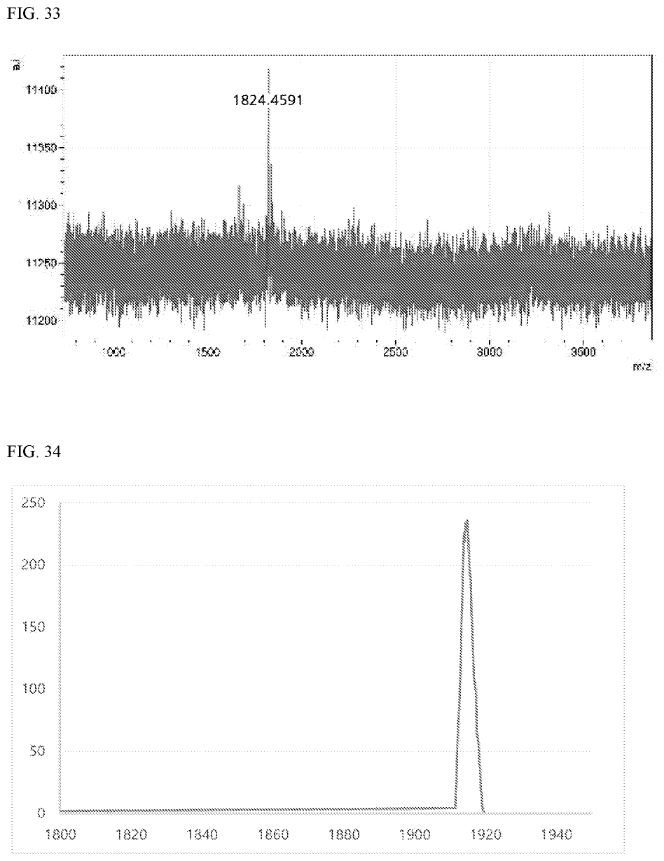

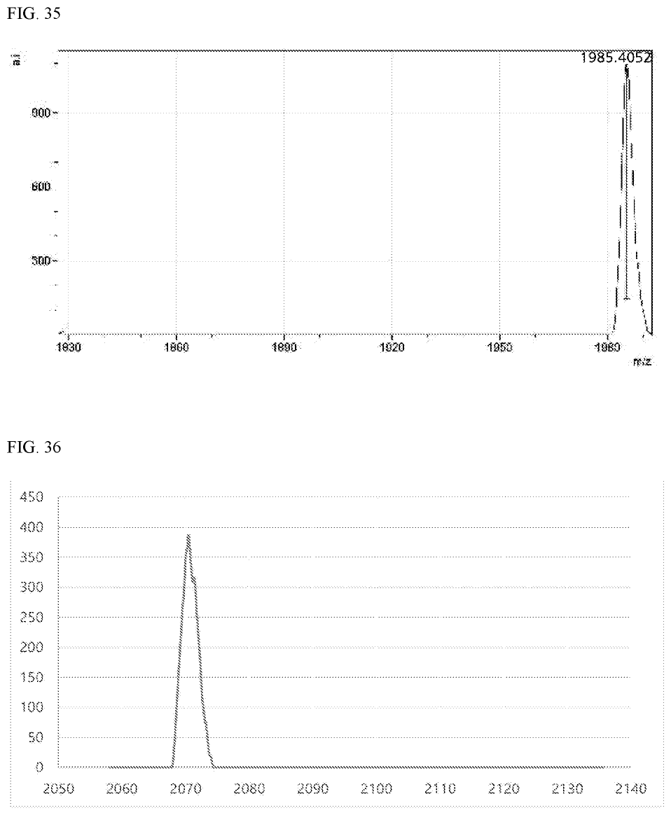

Coupling reagent such as HCTU (2-(6-chloro-1H-benzotriazole-1-yl)-1,1,3,3-tetramethylaminium hexafluorophosphate), HOBt (1-hydroxybenzotriazole) and Fmoc-PEG2-Suc-OH (Fmoc-ebes-OH) were purchased from AnaSpec (USA). The synthesis scale was typically 0.1 mmol and the synthesis was conducted mostly in a 6-mL Resprep SPE (solid-phase extraction) tube (Restek, USA). The synthesized peptides were purified by HPLC (high-performance liquid chromatography) using a C4 reversed-phase column (Waters, USA) at room temperature. The eluents were distilled water (0.1% TFA) and acetonitrile (0.1% TFA). The molecular weight of the peptides was measured using a MALDI-TOF (matrix-assisted laser desorption/ionization time-of-flight) mass spectrometer (FIGS. 1-36). The purity of the peptides was >95%, as determined by analytical HPLC.

### Examples 37-45. Preparation of Peptide Complexes (Wαₘ-RGD, Wαₘ-GS)

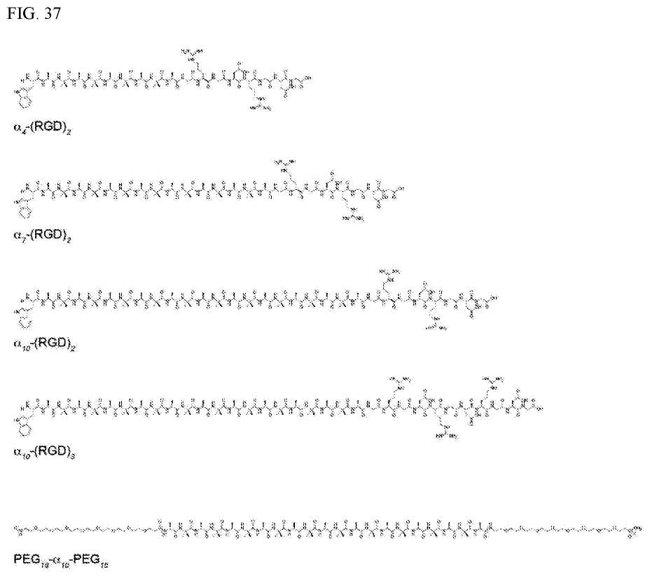

Peptide complexes represented by SEQ ID NOS 37-45 were prepared. Their structures are shown in FIG. 37. The peptide complexes were synthesized by standard Fmoc solid-phase peptide synthesis (SPPS) on Rink Amide MBHA resin LL (100-200 mesh, 0.30-0.40 mmol g⁻¹, Novabiochem, Germany) in the same manner as in Example 1.

The molecular weight of the peptide complexes was measured using a MALDI-TOF (matrix-assisted laser desorption/ionization time-of-flight) mass spectrometer. The purity of the peptide complexes was >95%, as determined by analytical HPLC.

### Examples 46-57. Preparation of Peptide Complexes (Wαₘ-PEG, PEG-αₘ)

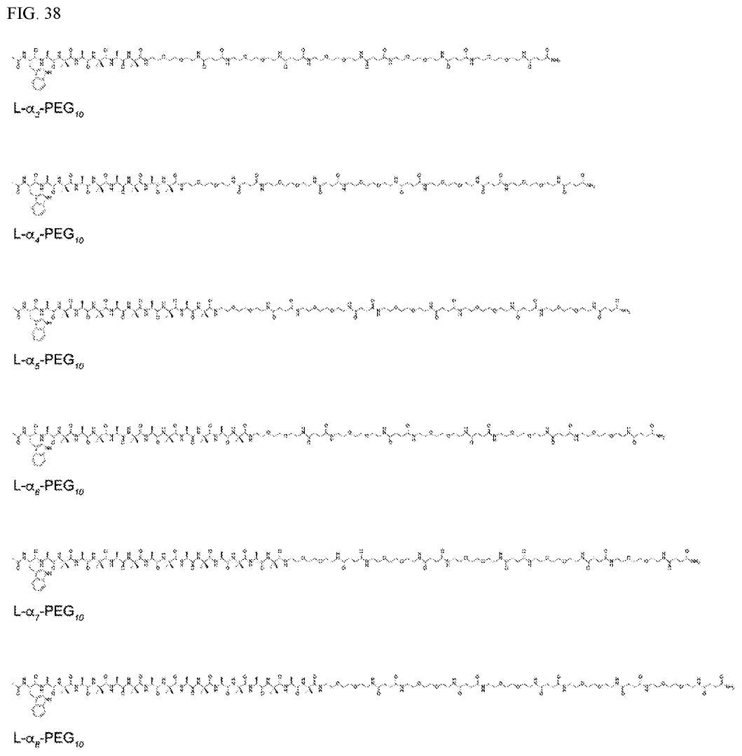

Peptide complexes of Example 46-57 wherein (PEG₂)₅ or (PEG₈)₂ is bound to peptides represented by SEQ ID NOS 2, 8, 14 and 46-51 were prepared by solid-phase peptide synthesis (Table 2). The structures of some of the peptide complexes are shown in FIG. 37 and FIG. 38. The solid-phase peptide synthesis was performed by standard Fmoc solid-phase peptide synthesis (SPPS) on Rink Amide MBHA resin LL (100-200 mesh, 0.30-0.40 mmol g⁻¹, Novabiochem, Germany) in the same manner as in Example 1.

TABLE 2

| | | | SEQ |
|---|---|---|---|
| | | | ID |
| | Name | Sequence | NO |
| 46 | L-α₄-PEG₁₀ | WAUAUAUAU(PEG2)₅ | 46 |
| 47 | L-α₅-PEG₁₀ | WAUAUAUAUAU(PEG2)₅ | 47 |
| 48 | L-α₆-PEG₁₀ | WAUAUAUAUAUAU(PEG2)₅ | 48 |
| 49 | L-α₇-PEG₁₀ | WAUAUAUAUAUAUAU(PEG2)₅ | 49 |
| 50 | L-α₈-PEG₁₀ | WAUAUAUAUAUAUAUAU(PEG2)₅ | 50 |
| 51 | α₈-(PEG2)₅ | WAUAUAUAUAUAUAUAUAU(PEG2)₅ | 51 |
| 52 | α₄-(PEG8)₂ | AUAUAUAUA(PEG8)₂ | 2 |
| 53 | α₇-(PEG8)₂ | AUAUAUAUAUAUAUA(PEG8)₂ | 8 |
| 54 | α₁₀-(PEG8)₂ | AUAUAUAUAUAUAUAUAUAUA(PEG8)₂ | 14 |
| 55 | (PEG8)₂-α₄ | (PEG8)₂ AUAUAUAUA | 2 |
| 56 | (PEG8)₂-α₇ | (PEG8)₂ AUAUAUAUAUAUAUA | 8 |
| 57 | (PEG8)₂-α₁₀ | (PEG8)₂ AUAUAUAUAUAUAUAUAUAUA | 14 |

### Examples 58-97. Preparation of Peptide Complexes (Rαₘ-PEG, Eαₘ-PEG)

Peptide complexes of Examples 58-97 wherein (PEG8)₂, (PEG8)₃, (PEG8)₄, (PEG8)₅ or glucose is bound to peptides represented by SEQ ID NOS 52-84 were prepared by solid-phase peptide synthesis (Table 3). The solid-phase peptide synthesis was performed by standard Fmoc solid-phase peptide synthesis (SPPS) on Rink Amide MBHA resin LL (100-200 mesh, 0.30-0.40 mmol g⁻¹, Novabiochem, Germany) in the same manner as in Example 1.

TABLE 3

| | Name | Sequence | SEQ ID NO |
|---|---|---|---|
| 58 | R-α₄-PEG₁₆ | RAUAUAUAUA(PEG8)₂ | 52 |
| 59 | R-α₇-PEG₁₆ | RAUAUAUAUAUAUAUA(PEG8)₂ | 53 |
| 60 | R-α₁₀-PEG₁₆ | RAUAUAUAUAUAUAUAUAUAUA(PEG8)₂ | 54 |
| 61 | R₂-α₁₀-PEG₁₆ | RRAUAUAUAUAUAUAUAUAUAUA(PEG8)₂ | 55 |
| 62 | R₃-α₁₀-PEG₁₆ | RRRAUAUAUAUAUAUAUAUAUAUA(PEG8)₂ | 56 |
| 63 | R₄-α₁₀-PEG₁₆ | RRRRAUAUAUAUAUAUAUAUAUAUA(PEG8)₂ | 57 |
| 64 | R-α₁₀-RGD | RAUAUAUAUAUAUAUAUAUAUARGDRGDRGD | 58 |
| 65 | R-α₁₀-RGD | RAUAUAUAUAUAUAUAUAUAUARGDRGDRGDR | 59 |
| | | GD | |
| 66 | R-α₁₀-RGD | RAUAUAUAUAUAUAUAUAUAUAGRGDRGDRGD | 60 |
| | | G | |
| 67 | R-α₁₀-RGD | RAUAUAUAUAUAUAUAUAUAUAGRGDRGDRGD | 61 |
| | | RGDG | |
| 68 | R₂-α₁₀-RGD | RRAUAUAUAUAUAUAUAUAUAUAGRGDRGDRG | 62 |
| | | DG | |
| 69 | R₃-α₁₀-RGD | RRRAUAUAUAUAUAUAUAUAUAUAGRGDRGDR | 63 |
| | | GDG | |
| 70 | RGD-α₁₀-R | RGDRGDAUAUAUAUAUAUAUAUAUAUAR | 64 |
| 71 | RGD-α₁₀-R | RGDRGDRGDAUAUAUAUAUAUAUAUAUAUAR | 65 |
| 72 | RGD-α₁₀-R | RGDRGDRGDRGDAUAUAUAUAUAUAUAUAUAU | 66 |
| | | AR | |
| 73 | RGD-α₁₀-R₂ | RGDRGDAUAUAUAUAUAUAUAUAUAUARR | 67 |
| 74 | RGD-α₁₀-R₂ | RGDRGDRGDAUAUAUAUAUAUAUAUAUAUARR | 68 |
| 75 | RGD-α₁₀-R₂ | RGDRGDRGDRGDAUAUAUAUAUAUAUAUAUAU | 69 |
| | | ARR | |
| 76 | R-α₁₀-PEG₁₆-g | RAUAUAUAUAUAUAUAUAUAUA(PEG8)₂-glucose | 54 |
| 77 | R₂-α₁₀-PEG₁₆-g | RRAUAUAUAUAUAUAUAUAUAUA(PEG8)₂- | 55 |
| | | glucose | |
| 78 | R₃-α₁₀-PEG₁₆-g | RRRAUAUAUAUAUAUAUAUAUAUA(PEG8)₂- | 56 |
| | | glucose | |
| 79 | R₄-α₁₀-PEG₁₆-g | RRRRAUAUAUAUAUAUAUAUAUAUA(PEG8)₂- | 57 |
| | | glucose | |
| 80 | g-PEG₁₆-α₁₀-R | Glucose-(PEG8)₂AUAUAUAUAUAUAUAUAUAUAR | 70 |
| 81 | g-PEG₂₄-α₁₀-R | Glucose-(PEG8)₃AUAUAUAUAUAUAUAUAUAUAR | 70 |
| 82 | g-PEG₂₄-α₁₀-R₂ | Glucose- | 71 |
| | | (PEG8)₃AUAUAUAUAUAUAUAUAUAUAUAUAUAR | |
| | | R | |
| 83 | g-PEG₃₂-α₁₀-R₂ | Glucose- | 71 |
| | | (PEG8)₄AUAUAUAUAUAUAUAUAUAUAUAUAUAR | |
| | | R | |
| 84 | g-PEG₃₂-α₁₀-R₂ | Glucose- | 72 |
| | | (PEG8)₄AUAUAUAUAUAUAUAUAUAUAUAUAUAU | |
| | | AUAUARR | |
| 85 | g-PEG₄₀-α₁₀-R₂ | Glucose- | 72 |
| | | (PEG8)₅AUAUAUAUAUAUAUAUAUAUAUAUAUAU | |
| | | AUAUARR | |
| 86 | g-GS-α₁₀-R₂ | Glucose-GSAUAUAUAUAUAUAUAUAUAUAR | 73 |
| 87 | g-GS₂-α₁₀-R₂ | Glucose-GSGSAUAUAUAUAUAUAUAUAUAUAR | 74 |
| 88 | g-GS₃-α₁₀-R₂ | Glucose- | 75 |
| | | GSGSGSAUAUAUAUAUAUAUAUAUAUAR | |
| 89 | g-GS₄-α₁₀-R₂ | Glucose- | 76 |
| | | GSGSGSGSAUAUAUAUAUAUAUAUAUAUAR | |
| 90 | E-α₁₀-PEG₁₆ | EAUAUAUAUAUAUAUAUAUAUA(PEG8)2 | 77 |
| 91 | E2-α₁₀-PEG₁₆ | EEAUAUAUAUAUAUAUAUAUAUA(PEG8)2 | 78 |
| 92 | E3-α₁₀-PEG₁₆ | EEEAUAUAUAUAUAUAUAUAUAUA(PEG8)2 | 79 |
| 93 | E4-α₁₀-PEG₁₆ | EEEEAUAUAUAUAUAUAUAUAUAUA(PEG8)2 | 80 |
| 94 | E-α₁₀-RGD | EAUAUAUAUAUAUAUAUAUAUARGDRGDRGD | 81 |
| 95 | E-α₁₀-GS | EAUAUAUAUAUAUAUAUAUAUAGSGSGSGS | 82 |
| 96 | g-PEG₁₆-α₁₀-E | Glucose-(PEG8)₂AUAUAUAUAUAUAUAUAUAUAE | 83 |
| 97 | g-PEG₁₆-α₁₀-E₂ | Glucose- | 84 |
| | | (PEG8)₂AUAUAUAUAUAUAUAUAUAUAEE | |

### Examples 98-111. Preparation of Cyclic Peptide Complexes (C-αₘ-PEG)

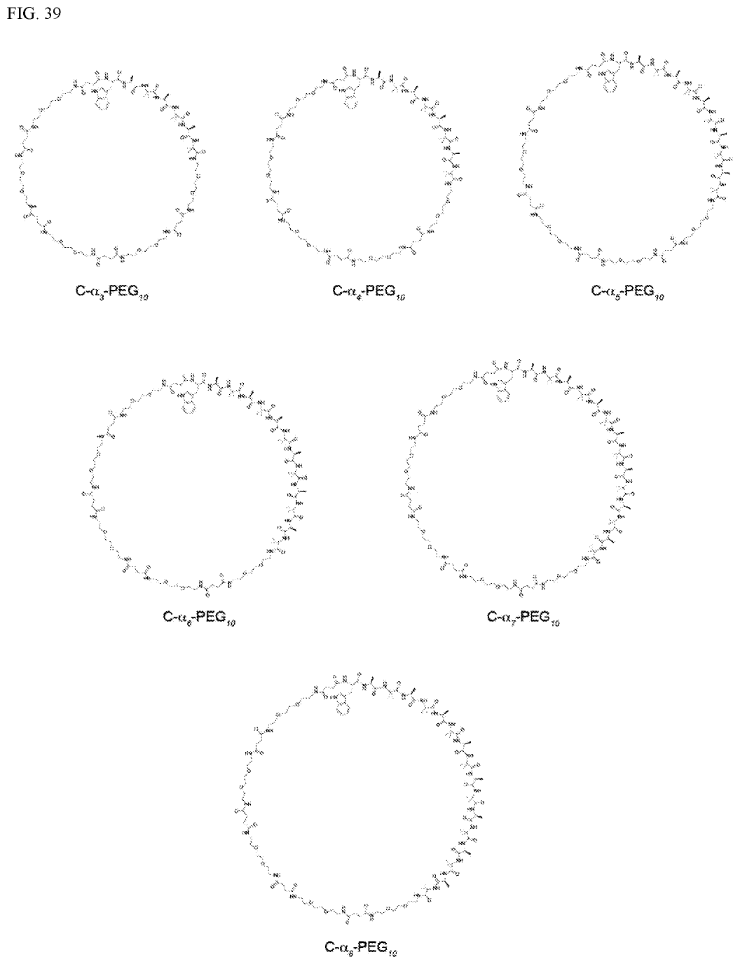

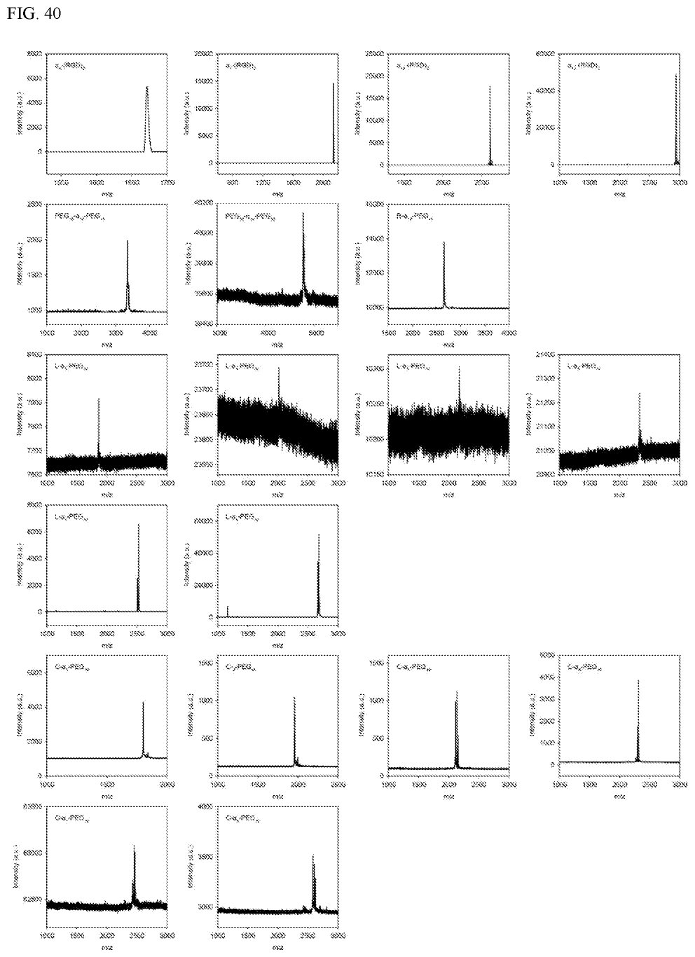

Peptide complexes of Examples 98-111, which have the same sequences as the peptide complexes of Examples 46-51 but are cyclic, were prepared by solid-phase peptide synthesis (Table 4). The structures of some of the cyclic peptide complexes are shown in FIG. 39. The solid-phase peptide synthesis was performed by standard Fmoc solid-phase peptide synthesis (SPPS) in the same manner as in Example 1 except that 2-chlorotrityl chloride resin (100-200 mesh) (Novabiochem, Germany) was used instead of the Rink Amide MBHA resin LL.

TABLE 4

| | Name | Sequence | SEQ ID NO |
|---|---|---|---|
| 98 | C-α₄-PEG₁₀ | WAUAUAUAU(PEG2)₅ | 46 |
| 99 | C-α₅-PEG₁₀ | WAUAUAUAUAU(PEG2)₅ | 47 |
| 100 | C-α₆-PEG₁₀ | WAUAUAUAUAUAU(PEG2)₅ | 48 |
| 101 | C-α₇-PEG₁₀ | WAUAUAUAUAUAUAU(PEG2)₅ | 49 |
| 102 | C-α₈-PEG₁₀ | WAUAUAUAUAUAUAUAU(PEG2)₅ | 50 |
| 103 | C-α₁₀-PEG₁₀ | WAUAUAUAUAUAUAUAUAU(PEG2)₅ | 51 |
| 104 | | WAUAUAUAUGHHHHHHHHHHGRGDRGDRGDG | 85 |
| 105 | | WAUAUAUAUAUGHHHHHHHHHHGRGDRGDRG | 87 |
| | | DG | |
| 106 | | WAUAUAUAUAUAUGHHHHHHHHHHGRGDRGD | 88 |
| | | RGDG | |
| 107 | | WAUAUAUAUAUAUAUGHHHHHHHHHHGRGDR | 89 |
| | | GDRGDG | |
| 108 | | WAUAUAUAUAUAUAUAUGHHHHHHHHHHGRG | 90 |
| | | DRGDRGDG | |
| 109 | | WAUAUAUAUAUAUAUAUAUGHHHHHHHHHHG | 91 |
| | | RGDRGDRGDG | |
| 110 | | WAUAUAUAUAUAUAUAU(PEG2)₅ | 50 + RGDRGD |
| | | RGDRGD(PEG2)₅ | |
| 111 | | (PEG2)₅ | 50 + RRRRRR |
| | | RRRRRR(PEG2)₅WAUAUAUAUAUAUAUAU | |

### Example 112-128. Preparation of Peptide Complexes (PEG-αₘ-PEG)

Peptide complexes of Examples 112-128 wherein (PEG8)₂, (PEG8)₃, (PEG10)₂ or (PEG10)₃ is bound to peptides represented by SEQ ID NOS 2, 8, 14 and 91-97 were prepared by solid-phase peptide synthesis (Table 5). The solid-phase peptide synthesis was performed by standard Fmoc solid-phase peptide synthesis (SPPS) on Rink Amide MBHA resin LL (100-200 mesh, 0.30-0.40 mmol g⁻¹, Novabiochem, Germany) in the same manner as in Example 1.

TABLE 5

| | Name | Sequence | SEQ ID NO |
|---|---|---|---|
| 112 | RGD-α₁₀-RGD | GRGDRGDRGDGWAUAUAUAUAUAUAUAUAUA | 91 |
| | | UAGRGDG | |
| 113 | RGD-α₁₀-RGD | GRGDGWAUAUAUAUAUAUAUAUAUAUAGRGD | 92 |
| | | RGDRGDG | |
| 114 | RGD-α₁₀-RGD | GRGDRGDGWAUAUAUAUAUAUAUAUAUAUAG | 93 |
| | | RGDRGDRGDG | |
| 115 | RGD-α₁₀-RGD | GRGDRGDRGDGWAUAUAUAUAUAUAUAUAUA | 94 |
| | | UAGRGDRGDRGDG | |
| 116 | GS-α₁₀-GS | GSGAUAUAUAUAUAUAUAUAUAUAGSG | 95 |
| 117 | GS-α₁₀-GS | GSGSGAUAUAUAUAUAUAUAUAUAUAGSGSG | 96 |
| 118 | GS-α₁₀-GS | GSGSGSGSGAUAUAUAUAUAUAUAUAUAUAGS | 97 |
| | | GSGSGSG | |
| 119 | PEG₈-α₄-PEG₁₆ | (PEG8)AUAUAUAUA(PEG8)₂ | 2 |
| 120 | PEG₈-α₇-PEG₁₆ | (PEG8)AUAUAUAUAUAUAUA(PEG8)₂ | 8 |
| 121 | PEG₈-α₁₀-PEG₁₆ | (PEG8)AUAUAUAUAUAUAUAUAUAUA(PEG8)₂ | 14 |
| 122 | PEG₁₆-α₁₀-PEG₈ | (PEG8)₂AUAUAUAUAUAUAUAUAUAUA(PEG8) | 14 |
| 123 | PEG₁₆-α₄-PEG₁₆ | (PEG8)₂AUAUAUAUA(PEG8)₂ | 2 |
| 124 | PEG₁₆-α₇-PEG₁₆ | (PEG8)₂AUAUAUAUAUAUAUA(PEG8)₂ | 8 |
| 125 | PEG₁₆-α₁₀-PEG₁₆ | (PEG8)₂AUAUAUAUAUAUAUAUAUAUA(PEG8)₂ | 14 |
| 126 | PEG₁₈-α₁₀-PEG₁₈ | (PEG8)₃AUAUAUAUAUAUAUAUAUAUA(PEG8)₃ | 14 |
| 127 | PEG₂₀-α₁₀-PEG₂₀ | (PEG10)₂ | 14 |
| | | AUAUAUAUAUAUAUAUAUAUA(PEG10)₂ | |
| 128 | PEG₃₀-α₁₀-PEG₃₀ | (PEG10)₃ | 14 |
| | | AUAUAUAUAUAUAUAUAUAUA(PEG10)₃ | |

### Test Example 1. MALDI-TOF Mass Spectrometry for Purified Peptides and Peptide Complexes

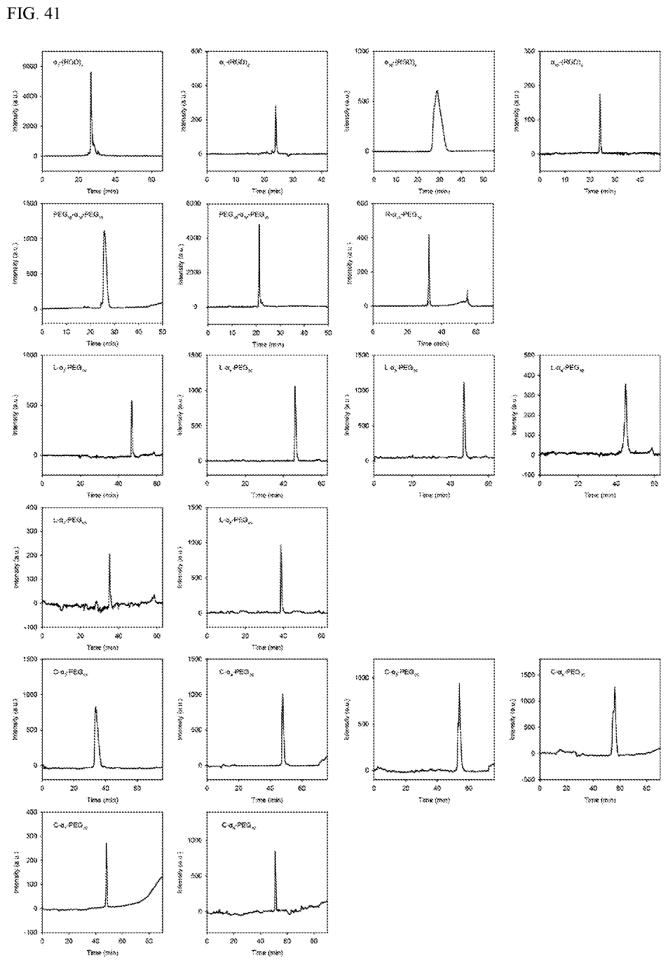

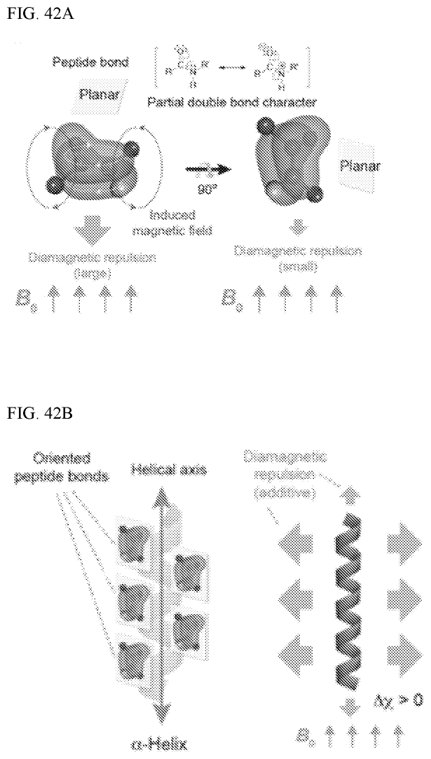

FIG. 40 shows the MALDI-TOF mass spectrometry result for the peptide complexes prepared in Examples 37, 39, 40, 46-50, 60, 98-102, 125 and 128 and FIG. 41 shows the HPLC chromatograms of the peptide complexes prepared in Examples 37, 39, 40, 46-50, 60, 98-102, 125 and 128.

As shown in FIGS. 40 and 41, it was confirmed that the peptide complexes of Examples 37, 39, 40, 46-50, 60, 98-102, 125 and 128 were synthesized successfully.

### Test Example 2. Designing and Analysis of Peptides Having MNP-Helix Rod Structure

Because the hydrophobic peptides of SEQ ID NOS 1-36 have high hydrophobicity, it is difficult to investigate their behaviors in an aqueous solvent. Therefore, peptide complexes with a hydrophilic material bound to one end of the hydrophobic peptides were used for analysis. That is to say, the peptide complexes prepared in Examples 37-40 (αₘ-(RGD)₂) were analyzed by CD spectroscopy.

CD (circular dichroism) spectroscopy was performed as follows. The peptide complexes prepared in Examples 37-40 (αₘ-(RGD)₂) were prepared at various concentrations (0.156-160 μM in H₂O) and analyzed using a Chirascan CD spectrometer (Applied Photophysics, UK. A cuvette with a path length of 2 mm was used. Each scan was repeated three times and averaged. MRE (mean residue ellipticity) was calculated based on the number of amino acid residues.

FIG. 42a schematically shows the diamagnetic anisotropy of the peptide bond of the hydrophobic peptide according to the present disclosure, wherein B₀ represents external magnetic field (MF). FIG. 42b shows the α-helix structure and helical axis of the hydrophobic peptide according to the present disclosure. The aligned peptide bonds are shown and the difference in the diamagnetic susceptibility between the parallel and perpendicular directions is shown schematically.

FIG. 42c shows the rod-coil structure of the peptide complexes prepared in Examples 37-41 (αₘ-RGD). The peptide complex according to the present disclosure has a rod-coil structure formed as a hydrophobic peptide including a repeat unit (a) consisting of two amino acid residues binds to a hydrophilic peptide (RGD). FIG. 42d shows the CD spectra of the peptide complexes prepared in Examples 37, 39 and 40 (α₄-RGD₂, α₇-RGD₂ and α₁₀-RGD₂).

As shown in FIG. 42, it can be seen that the peptide complexes prepared in Examples 37-41 (αₘ-RGD) were formed as the rod portion having the α-helix structure was successfully bound to the portion having the coil structure. In particular as the number of repeat sequences (a) consisting of U and A increased (Example 37→40), the α-helix structure of the peptide complexes also increased gradually. Specifically, the peptide complex prepared in Example 40(α₁₀-RGD₂) had the most perfect α-helix structure.

### Test Example 3. Structural Analysis of Peptide Complexes

An amphiphilic molecule is a molecule consisting of a hydrophilic moiety and a hydrophobic moiety. The amphiphilic molecules can associate in solution due to hydrophobic effect to form self-assembled structures. The peptide complexes prepared in Examples 37, 39 and 40 (α₄-RGD₂, α₇-RGD₂, α₁₀-RGD₂) of the present disclosure are amphiphilic molecules. Although it was confirmed in Test Example 2 that the peptide complexes prepared in Examples 37, 39 and 40 have the α-helix structure, this result only means that the α-helix structure is stabilized in the peptide complexes and it could not be confirmed whether this was due to the stabilized α-helix structure in the hydrophobic peptide or due to the stabilization of the helix structure through binding to the hydrophilic moiety (RGD₂).

Therefore, the structure of the peptide complex prepared in Example 125 was analyzed (CD spectroscopy and AFM measurement). Since the peptide complex prepared in Example 125 has hydrophilic moieties bound to both ends of the hydrophobic peptide, the possibility of the stabilization of the α-helix structure through structural restraint via self-assembly is excluded. The peptide complex prepared in Example 125 had a coil-rod-coil structure.

The CD spectroscopy was performed in the same manner as in Test Example 2 and the AFM was performed using the NX10 system (Park Systems, Korea) in a non-contact mode with PPP-NCHR AFM probes (Nanosensors, Switzerland). The peptide complex concentration ranged from 1 to 32 μM (in H₂O). 2 μL of the sample solution was cast onto a freshly cleaved mica surface and dried. The data obtained by the SmartScan program (Park Systems, Korea) were analyzed using the XEI program (Park Systems, Korea).

FIG. 42e shows the structure of the peptide complex prepared in Example 125 (PEG₁₆-α₁₀-PEG₁₆). FIG. 42f shows the CD spectra of the peptide complex prepared in Example 125 depending on concentration and FIG. 42g shows a result of measuring the helicity of the peptide complex prepared in Example 125 depending on concentration.

As shown in FIG. 42e, the peptide complex of Example 125 existed in monomeric state having the coil-rod-coil structure and the hydrophobic peptide had a rod structure with the α-helix secondary structure.

As shown in FIG. 42f and FIG. 42g, two distinct peaks corresponding to the α-helix structure were observed at 208 nm and 222 nm for the peptide complex of Example 125 (PEG₁₆-α₁₀-PEG₁₆).

The [θ]₂₂₂/[θ]₂₀₃ ratio of the peptide complex of Example 125 (PEG₁₆-α₁₀-PEG₁₆) was maintained at 1.1-1.2, indicating that the peptide complex of Example 125 (PEG₁₆-α₁₀-PEG₁₆) has a perfect α-helix structure and exists in monomeric state without self-assembly.

Typically, a [θ]₂₂₂/[θ]₂₀₃ ratio higher than 1.0 is regarded as a coiled coil structure in which the α-helix structure is stabilized. However, because the peptide complex of the present disclosure has a ratio of 1.0 or lower, it can be seen that the α-helix structure exists independently.

FIG. 43 shows the AFM images of the peptide complexes prepared in Examples 37, 39, 40 and 125 (a, b, c, d; α₄-(RGD)₂, α₇-(RGD)₂, α₁₀-(RGD)₂, PEG₁₆-α₁₀-PEG₁₆) for comparing the self-assembly behavior of the peptide complex having a rod-coil structure and the peptide complex having a coil-rod-coil structure.

The peptide complexes of Examples 37, 39 and 40 are peptide complexes having the rod-coil structure. They self-assembled into spherical particles such as micelles in an aqueous solvent. In contrast, the peptide complex of Example 125 has a coil-rod-coil structure and did not self-assemble in an aqueous solvent. That is to say, it can be seen that the α-helices of the peptide complex of Example 125 do not interact with each other.

From these results, it was confirmed that α₁₀ (Example 14; SEQ ID NO 14) consisting of the shortest repeat unit has a monomeric, nonpolar and perfect α-helix (MNP-helix) structure.

### Test Example 4. Structural Analysis of Peptide Alignment Under Magnetic Field

Anisotropic diamagnetic molecules placed in an isotropic solution are aligned when a magnetic field is applied. The behavior of the peptide according to the present disclosure under a magnetic field was analyzed. For this, RDCs (residual dipolar couplings) between ¹H-¹⁵N amide pairs (¹D_{NH}) were measured by NMR (nuclear magnetic resonance) at 16.45 T (700.400 MHz) and 21.14 T (900.230 MHz).

The peptide complex of Example 128 (PEG₃₀-α₁₀-PEG₃₀) was used as a sample. The NMR sample was prepared by dissolving in a methanol-d₃/chloroform-d (1:1) mixture containing 0.05% TFA to a concentration of 4.26 mM.

Specifically, ¹H NMR and ²D ¹H-¹⁵N IPAP-HSQC (in-phase/anti-phase heteronuclear single quantum coherence) NMR spectra were analyzed at 298 K using a BrukerAVANCE IV 900 MHz spectrometer and a BrukerAVANCE Ill HD 700 MHz spectrometer equipped with cryogenic probes. The NMR sample was prepared by dissolving in a methanol-d₃/chloroform-d (1:1) mixture containing 0.05% TFA to a concentration of 4.26 mM. The acquisition parameters of IPAP were 2048 t₂×1024 t₁ points, 8 scans, 0.2 second recycle delay, 14 ppm spectral width for ¹H and 26 ppm spectral width for ¹⁵N. The acquired data were split into two data sets, IP and AP, using the TopSpin software (Bruker, Germany). Each spectrum was processed by zero-filling up to 8192×8192 points. Peak positions were analyzed using the NMRFAM-SPARKY software.

Magnetic isotropy can be defined by Equation 1.

$$Δχ = χΠ - χ ⊥ \qquad [ Equation 1 ]$$

wherein
χ^{II} is the magnetic susceptibility of the long axis, and
χ^{⊥} is the magnetic susceptibility of the short axis.

If Δ_{χ} is positive, the molecule will align with the direction of the magnetic field and vice versa. It is known that α-helices are aligned parallel to the external magnetic field because Δ_{χ} is positive.

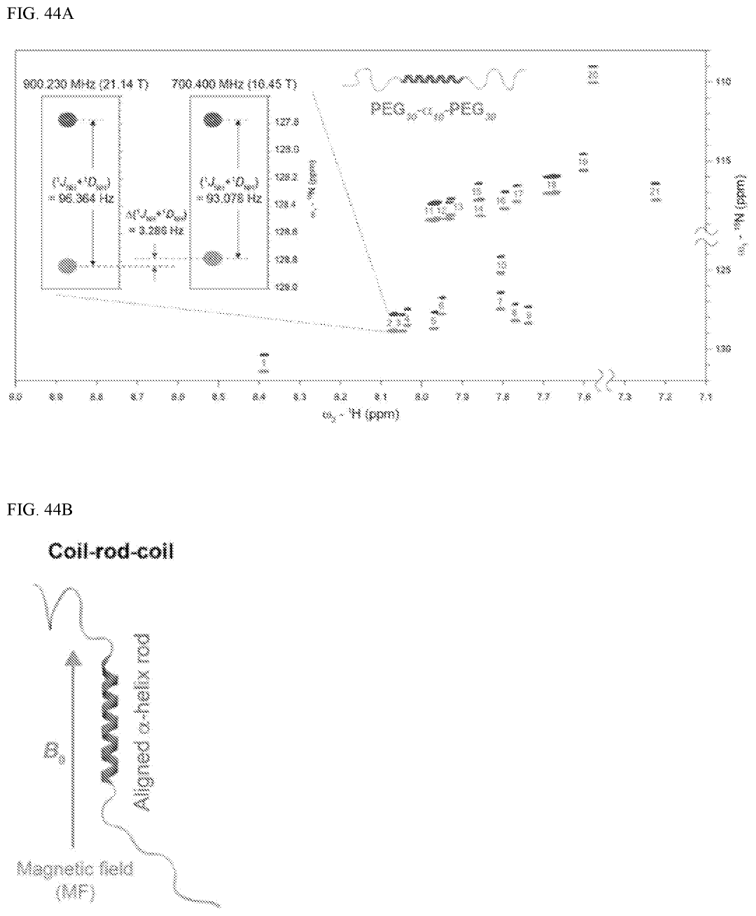

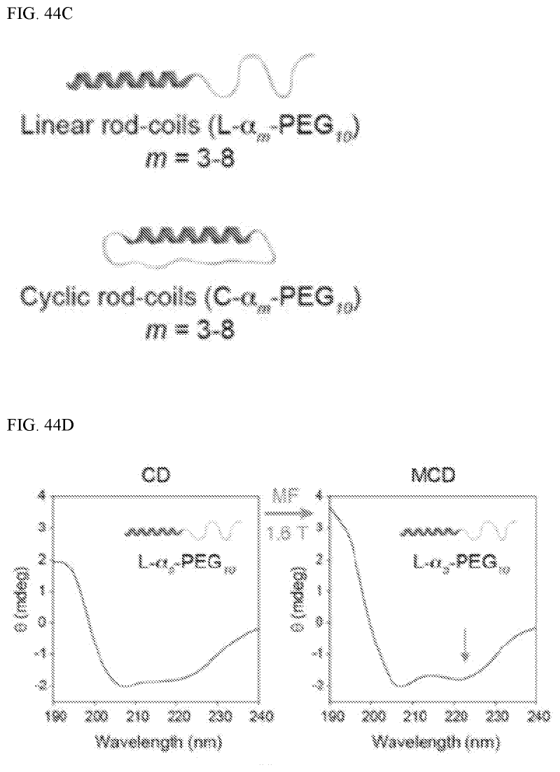

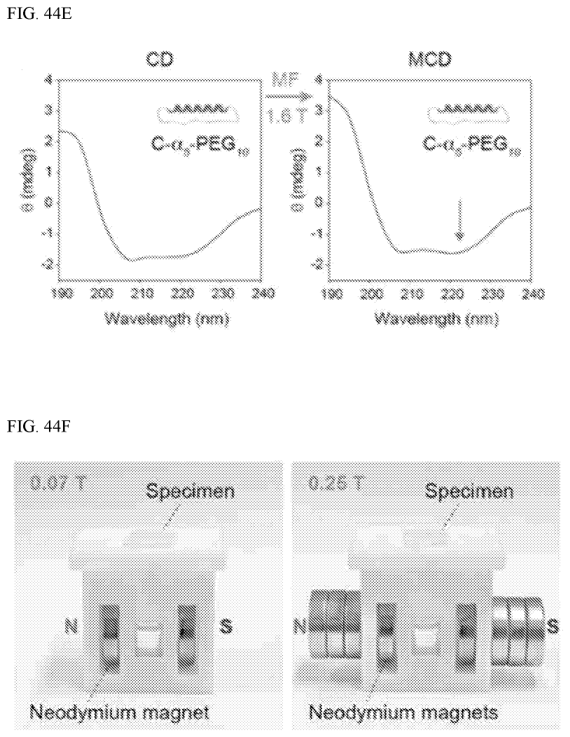

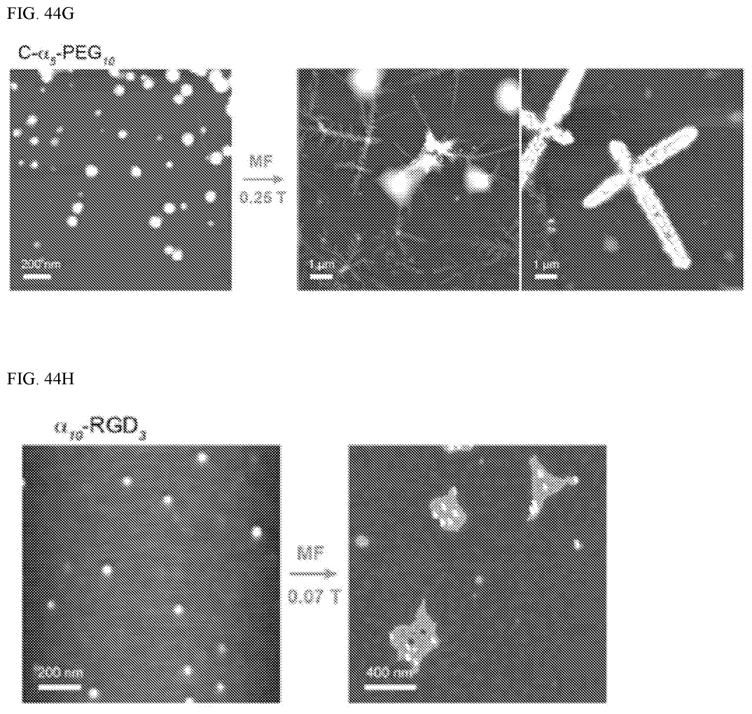

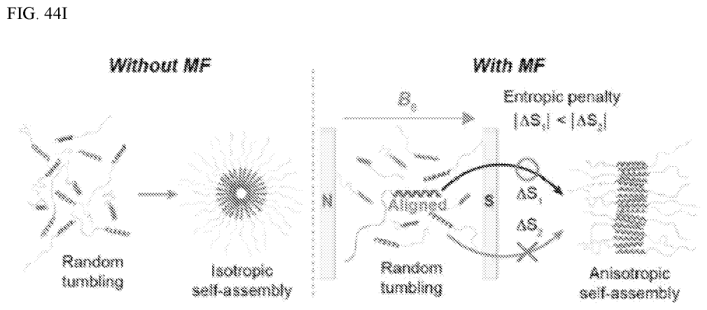

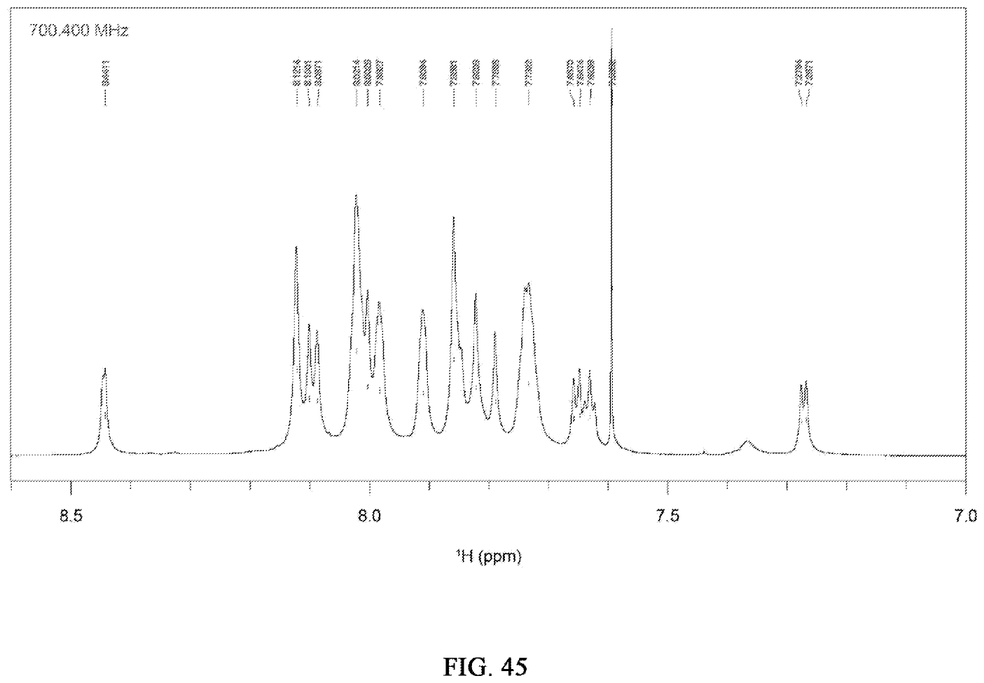

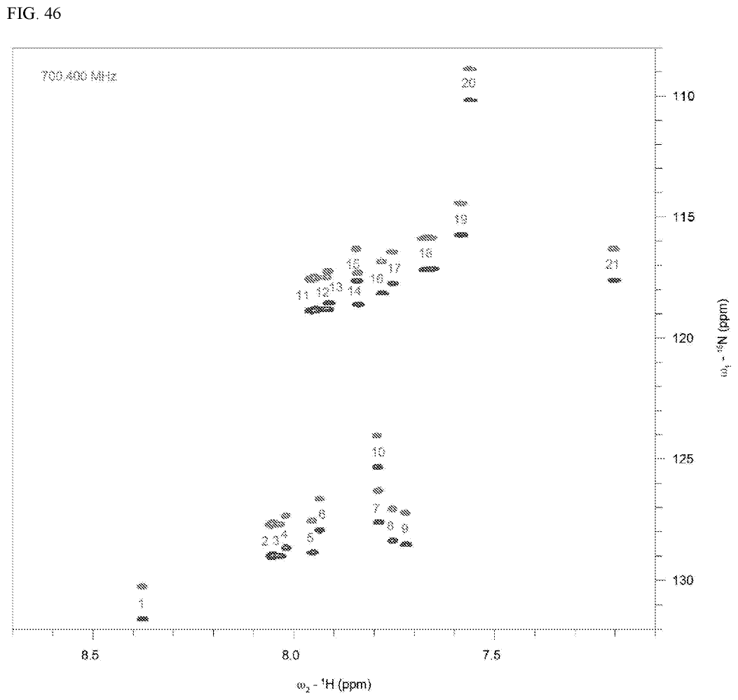

N-H couplings (¹J_{NH}+¹D_{NH}) for 21 amino acid residues of the peptide of SEQ ID NO 10 (α₁₀) in the peptide complex of Example 128 (PEG₃₀-α₁₀-PEG₃₀) according to the present disclosure were measured. The result is shown in FIG. 44a, FIGS. 45, 46, 47 and 48 and Tables 1 and 2.

FIG. 44a shows the ¹H-¹⁵N IPAP-HSQC spectrum of the peptide complex prepared in Example 128 (PEG₃₀-α₁₀-PEG₃₀) and FIG. 44b schematically shows the behavior of the peptide complex prepared in Example 128 (PEG₃₀-α₁₀-PEG₃₀) under a magnetic field.

FIG. 45 shows the ¹H NMR spectrum of the peptide complex prepared in Example 128 (PEG₃₀-α₁₀-PEG₃₀) measured at 700.400 MHz (16.45 T) and FIG. 46 shows the ¹H-¹⁵N IPAP-HSQC spectrum of the peptide complex prepared in Example 128 (PEG₃₀-α₁₀-PEG₃₀) measured at 700.400 MHz (16.45 T). Table 6 shows the ¹J_{NH}+¹D_{NH} splitting of the peptide complex of Example 128 (PEG₃₀-α₁₀-PEG₃₀) measured at 700.400 MHz (16.45 T).

TABLE 6

| | ¹H | | ¹⁵N | | ¹J_{NH} + ¹D_{NH} |
|---|---|---|---|---|---|
| 700.400 MHz | (ppm) | (Hz) | (ppm) | (Hz) | (Hz) |
| 1 | 8.379 | 594.892 | 130.234 | 9246.354 | 93.149 |
| | 8.378 | 594.821 | 131.546 | 9339.503 | |
| 2 | 8.057 | 572.031 | 127.645 | 9062.540 | 93.078 |
| | 8.056 | 571.960 | 128.956 | 9155.618 | |
| 3 | 8.035 | 570.469 | 127.661 | 9063.676 | 93.007 |
| | 8.035 | 570.469 | 128.971 | 9156.683 | |
| 4 | 8.022 | 569.546 | 127.309 | 9038.684 | 92.794 |
| | 8.021 | 569.475 | 128.616 | 9131.479 | |
| 5 | 7.957 | 564.931 | 127.514 | 9053.239 | 92.794 |
| | 7.955 | 564.789 | 128.821 | 9146.033 | |
| 6 | 7.938 | 563.582 | 126.592 | 8987.779 | 92.510 |
| | 7.937 | 563.511 | 127.895 | 9080.289 | |
| 7 | 7.793 | 553.287 | 126.271 | 8964.988 | 92.581 |
| | 7.791 | 553.145 | 127.575 | 9057.570 | |
| 8 | 7.757 | 550.731 | 127.027 | 9018.663 | 92.084 |
| | 7.756 | 550.660 | 128.324 | 9110.747 | |
| 9 | 7.724 | 548.389 | 127.182 | 9029.668 | 92.581 |
| | 7.723 | 548.318 | 128.486 | 9122.249 | |
| 10 | 7.796 | 553.500 | 123.999 | 8803.681 | 92.084 |
| | 7.794 | 553.358 | 125.296 | 8895.765 | |
| 11 | 7.958 | 565.002 | 117.523 | 8343.898 | 93.717 |
| | 7.957 | 564.931 | 118.843 | 8437.615 | |
| 12 | 7.920 | 562.304 | 117.445 | 8338.360 | 94.001 |
| | 7.919 | 562.233 | 118.769 | 8432.361 | |
| 13 | 7.916 | 562.020 | 117.202 | 8321.108 | 93.717 |
| | 7.914 | 561.878 | 118.522 | 8414.825 | |
| 14 | 7.843 | 556.837 | 117.266 | 8325.651 | 93.291 |
| | 7.842 | 556.766 | 118.580 | 8418.943 | |
| 15 | 7.847 | 557.121 | 116.276 | 8255.363 | 93.646 |
| | 7.845 | 556.979 | 117.595 | 8349.010 | |
| 16 | 7.784 | 552.648 | 116.804 | 8292.850 | 93.149 |
| | 7.782 | 552.506 | 118.116 | 8386.000 | |
| 17 | 7.757 | 550.731 | 116.408 | 8264.735 | 92.510 |
| | 7.756 | 550.660 | 117.711 | 8357.246 | |
| 18 | 7.671 | 544.626 | 115.840 | 8224.408 | 92.439 |
| | 7.669 | 544.484 | 117.142 | 8316.848 | |
| 19 | 7.588 | 538.733 | 114.392 | 8121.603 | 92.723 |
| | 7.586 | 538.591 | 115.698 | 8214.327 | |
| 20 | 7.565 | 537.100 | 108.835 | 7727.067 | 92.652 |
| | 7.564 | 537.029 | 110.140 | 7819.720 | |
| 21 | 7.207 | 511.683 | 116.274 | 8255.221 | 92.226 |
| | 7.206 | 511.612 | 117.573 | 8347.448 | |

| | |
|---|---|
| Average | 92.892 |

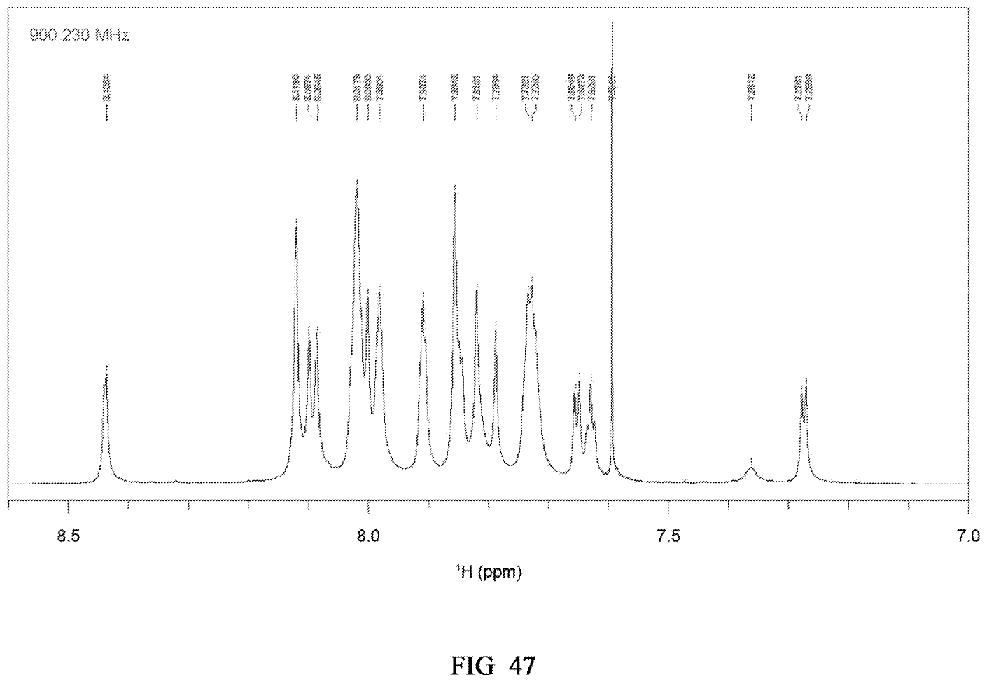

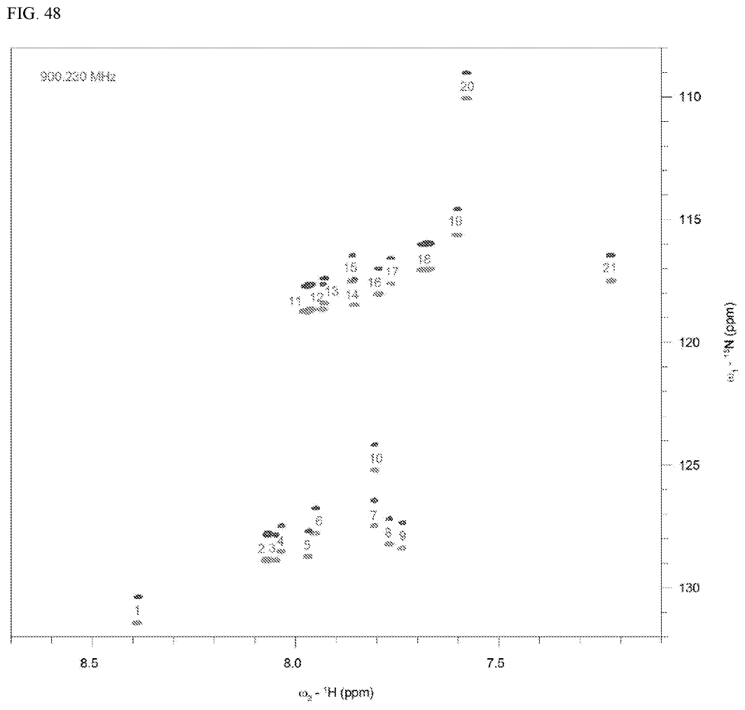

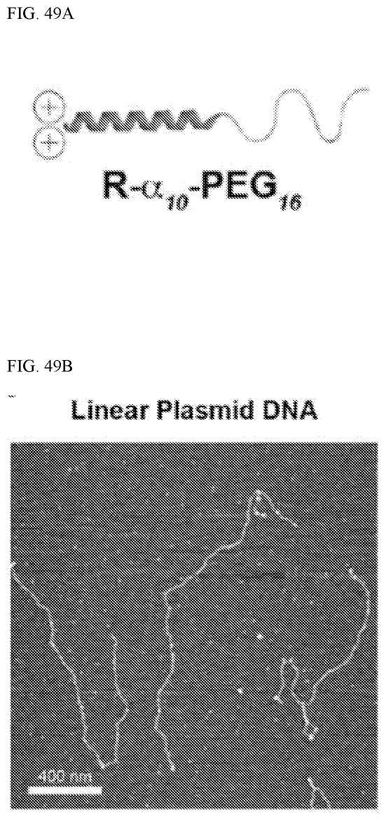

FIG. 47 shows the ¹H NMR spectrum of the peptide complex prepared in Example 128 (PEG₃₀-α₁₀-PEG₃₀) measured at 900.230 MHz (21.14 T) and FIG. 48 shows the ¹H-¹⁵N IPAP-HSQC spectrum of the peptide complex prepared in Example 128 (PEG₃₀-α₁₀-PEG₃₀) measured at 900.230 MHz (21.14 T). Table 7 shows the ¹J_{NH}+¹D_{NH} splitting of the peptide complex of Example 128 (PEG₃₀-α₁₀-PEG₃₀) measured at 900.230 MHz (21.14 T).

TABLE 7

| | ¹H | | ¹⁵N | | ¹J_{NH} + ¹D_{NH} |
|---|---|---|---|---|---|
| 900.230 MHz | (ppm) | (Hz) | (ppm) | (Hz) | (Hz) |
| 1 | 8.386 | 765.256 | 130.351 | 11895.050 | 95.087 |
| | 8.388 | 765.439 | 131.393 | 11990.137 | |
| 2 | 8.068 | 736.237 | 127.769 | 11659.432 | 96.364 |
| | 8.069 | 736.329 | 128.825 | 11755.797 | |
| 3 | 8.046 | 734.230 | 127.798 | 11662.079 | 94.813 |
| | 8.047 | 734.321 | 128.837 | 11756.892 | |
| 4 | 8.033 | 733.043 | 127.439 | 11629.319 | 94.813 |
| | 8.034 | 733.135 | 128.478 | 11724.131 | |
| 5 | 7.967 | 727.021 | 127.641 | 11647.752 | 94.995 |
| | 7.968 | 727.112 | 128.682 | 11742.747 | |
| 6 | 7.949 | 725.378 | 126.715 | 11563.251 | 95.087 |
| | 7.950 | 725.469 | 127.757 | 11658.337 | |
| 7 | 7.804 | 712.146 | 126.400 | 11534.506 | 94.904 |
| | 7.805 | 712.237 | 127.440 | 11629.410 | |
| 8 | 7.768 | 708.861 | 127.150 | 11602.946 | 94.083 |
| | 7.768 | 708.861 | 128.181 | 11697.029 | |
| 9 | 7.736 | 705.941 | 127.311 | 11617.638 | 94.813 |
| | 7.736 | 705.941 | 128.350 | 11712.451 | |
| 10 | 7.803 | 712.055 | 124.138 | 11328.089 | 94.083 |
| | 7.804 | 712.146 | 125.169 | 11422.172 | |
| 11 | 7.972 | 727.477 | 117.658 | 10736.763 | 95.087 |
| | 7.972 | 727.477 | 118.700 | 10831.850 | |
| 12 | 7.932 | 723.827 | 117.578 | 10729.463 | 95.360 |
| | 7.932 | 723.827 | 118.623 | 10824.823 | |
| 13 | 7.928 | 723.462 | 117.335 | 10707.288 | 95.360 |
| | 7.928 | 723.462 | 118.380 | 10802.649 | |
| 14 | 7.854 | 716.709 | 117.394 | 10712.672 | 95.452 |
| | 7.855 | 716.800 | 118.440 | 10808.124 | |
| 15 | 7.858 | 717.074 | 116.406 | 10622.513 | 95.634 |
| | 7.859 | 717.165 | 117.454 | 10718.147 | |
| 16 | 7.794 | 711.234 | 116.933 | 10670.604 | 95.178 |
| | 7.795 | 711.325 | 117.976 | 10765.782 | |
| 17 | 7.763 | 708.405 | 116.525 | 10633.372 | 93.992 |
| | 7.764 | 708.496 | 117.555 | 10727.364 | |
| 18 | 7.679 | 700.739 | 115.952 | 10581.084 | 94.630 |
| | 7.680 | 700.831 | 116.989 | 10675.714 | |
| 19 | 7.600 | 693.530 | 114.526 | 10450.956 | 94.630 |
| | 7.601 | 693.622 | 115.563 | 10545.586 | |
| 20 | 7.578 | 691.523 | 108.969 | 9943.857 | 94.083 |
| | 7.578 | 691.523 | 110.000 | 10037.940 | |
| 21 | 7.224 | 659.219 | 116.401 | 10622.057 | 94.448 |
| | 7.223 | 659.128 | 117.436 | 10716.505 | |

| | |
|---|---|
| Average | 94.900 |

Table 8 shows the experimental RDCs (¹D_{NH, exp}) of the peptide complex of Example 128 (PEG₃₀-α₁₀-PEG₃₀). The experimental RDCs are calculated from Equation 2.

$$( {}_{}^{1} {D}_{NH , exp}^{} ) = {( {}_{}^{1} {J}_{NH}^{} + {}_{}^{1} {D}_{NH}^{} )}^{( 21.14 T )} - {( {}_{}^{1} {J}_{NH}^{} + {}_{}^{1} {D}_{NH}^{} )}^{( 16.45 T )} \qquad [ Equation 2 ]$$

TABLE 8

| | (¹J_{NH} + ¹D_{NH})^{21.14 T} | (¹J_{NH} + ¹D_{NH})^{16.45 T} | ¹D_{NH, exp} |
|---|---|---|---|
| 1 | 95.087 | 93.149 | 1.937 |
| 2 | 96.364 | 93.078 | 3.286 |
| 3 | 94.813 | 93.007 | 1.806 |
| 4 | 94.813 | 92.794 | 2.019 |
| 5 | 94.995 | 92.794 | 2.201 |
| 6 | 95.087 | 92.510 | 2.576 |
| 7 | 94.904 | 92.581 | 2.323 |
| 8 | 94.083 | 92.084 | 1.998 |
| 9 | 94.813 | 92.581 | 2.232 |
| 10 | 94.083 | 92.084 | 1.998 |
| 11 | 95.087 | 93.717 | 1.369 |
| 12 | 95.360 | 94.001 | 1.359 |
| 13 | 95.360 | 93.717 | 1.643 |
| 14 | 95.452 | 93.291 | 2.160 |
| 15 | 95.634 | 93.646 | 1.988 |
| 16 | 95.178 | 93.149 | 2.029 |
| 17 | 93.992 | 92.510 | 1.481 |
| 18 | 94.630 | 92.439 | 2.191 |
| 19 | 94.630 | 92.723 | 1.907 |
| 20 | 94.083 | 92.652 | 1.430 |
| 21 | 94.448 | 92.226 | 2.221 |
| Average | 94.900 | 92.892 | 2.007 |

As shown in FIGS. 44-48 and Table 8, the peptide complex of Example 128 (PEG₃₀-α₁₀-PEG₃₀) has positive RDC (¹D_{NH, exp}) values. Because the dipolar coupling (¹D) correlates positively with Δ_{χ}, it indicates that the peptide complex of Example 128 (PEG₃₀-α₁₀-PEG₃₀) aligns in the direction of the external magnetic field.

The magnitude of the average RDC value (2.007 Hz) of the α₁₀ peptide in the peptide complex of Example 128 is fairly high for an organic molecule. These results reveal that the peptide bonds in all 21 residues of the α₁₀ peptide (SEQ ID NO 14) are involved in the formation of the α-helix structure.

Taken together, it can be seen that the hydrophobic peptides of the present disclosure (SEQ ID NOS 1-36) have superior magnetic responsiveness and the superior responsiveness to a magnetic field can be maintained even when the hydrophobic peptides are prepared into amphiphilic or hydrophilic molecules through binding with a polymer exhibiting hydrophilicity or a peptide exhibiting hydrophilicity.

### Test Example 5. Control of Self-Assembly of Peptide Complexes by Magnetic Field

In order to investigate whether the self-assembly process of the peptide complexes according to the present disclosure can be controlled with a magnetic field, the peptide complexes of Examples 46-50 having a linear rod-coil structure and the peptide complexes of Examples 98-102 having a cyclic rod-coil structure were prepared. The chemical structures of the peptide complexes of Examples 46-50 and the peptide complexes of Examples 98-102 are compared in FIG. 44c. In addition, the change of the α-helix structure was investigated by measuring CD spectra and MCD spectra and the morphological change under a magnetic field was investigated by AFM analysis.

The CD spectra were analyzed using a Chirascan CD spectrometer (Applied Photophysics, UK). The peptide complex concentration ranged from 0.156 to 160 μM. All the samples were dissolved in H₂O. Each scan was repeated three times using a 2-mm path-length cuvette and averaged. MRE (mean residue ellipticity) was calculated based on the number of amino acid residues.

MCD (magnetic circular dichroism) spectra were obtained using a Jasco-815 159-L spectrophotometer (Jasco, Japan). The concentration of the peptide complex was 20 μM in distilled water. Measurements were performed using a 2-mm path-length cuvette. The magnetic field of the permanent magnet in the MCD accessory (PM-491) was 1.6 T. Each scan was repeated three times and averaged.

AFM was performed using the NX10 system (Park Systems, Korea) in a non-contact mode with PPP-NCHR AFM probes (Nanosensors, Switzerland). The peptide complex (specimen) dispersed in an aqueous solvent was placed between neodymium magnets and measurements were compared before and after the application of a magnetic field. The magnetic field intensity was measured using a Model 450 gaussmeter (Lake Shore Cryotronics, USA).

FIG. 44c schematically shows the difference in the chemical structure of the peptide complex of Example 47 and the peptide complex of Example 99. FIG. 44d shows the CD and MCD (magnetic circular dichroism) spectra of the peptide complex of Example 47 (L-α₅-PEG₁₀). FIG. 44e shows the CD and MCD (magnetic circular dichroism) spectra of the peptide complex of Example 99 (C-α₅-PEG₁₀). Table 9 shows the result of analyzing the morphological characteristics of the peptide complexes prepared in Examples 46-50 (L-α₄₋₈-PEG₁₀) and the peptide complexes prepared in Examples 98-102 (C-α₄₋₈-PEG₁₀) measured by CD spectroscopy.

TABLE 9

| Name | [θ]₂₂₂/[θ]₂₀₈ |
|---|---|
| L-a₃-PEG₁₀ | n.d.^{a} |
| Example 46 | n.d. |
| L-a₄-PEG₁₀ | |
| Example 47 | 0.84 |
| L-a₅-PEG₁₀ | |
| Example 48 | 0.88 |
| L-a₆-PEG₁₀ | |
| Example 49 | 1.05 |
| L-a₇-PEG₁₀ | |
| Example 50 | 1.10 |
| L-a₈-PEG₁₀ | |
| C-a₃-PEG₁₀ | n.d. |
| Example 98 | n.d. |
| C-a₄-PEG₁₀ | |
| Example 99 | 0.93 |
| C-a₅-PEG₁₀ | |
| Example 100 | 0.95 |
| C-a₆-PEG₁₀ | |
| Example 101 | 1.01 |
| C-a₇-PEG₁₀ | |
| Example 102 | 1.10 |
| C-a₈-PEG₁₀ | |

^{a}n.d. = not determined

As shown in FIG. 44c and Table 9, the peptide complexes of Examples 46-50 (L-αₘ PEG₁₀) and the peptide complexes of Examples 98-102 (C-α₄₋₈-PEG₁₀) showed gradual increase of α-helices as the length of the α peptide increases.

MCD (magnetic circular dichroism) analysis was performed to investigate whether the helical stability of the αₘ peptide in the peptide complex according to the present disclosure is affected by the magnetic field. The peptide complexes of Examples 47 and 99 have partially stable α-helices. When a magnetic field of 1.6 T was applied to the peptide complexes of Examples 47 and 99 (FIGS. 44d, 44e), the contents of α-helices, i.e. the [θ]₂₂₂/[6]₂₀₈ ratios, were increased in both molecules. That is to say, it was confirmed that the conformational change of the α-helix structure of the peptide complex according to the present disclosure can be induced with a magnetic field.

The extent of the increase in the [6]₂₂₂/[θ]₂₀₈ ratio was higher for the peptide complex of Example 99 (C-α₅-PEG₁₀) (from 0.89 to 1.01) than for the peptide complex of Example 47 (L-α₅-PEG₁₀) (from 0.86 to 0.91). This result suggests that the cyclic peptide complex responds more sensitively to a magnetic field than the linear peptide complex. That is to say, it was confirmed that the sensitivity to a magnetic field can be increased by structural difference even for the same sequence.

FIG. 44f shows the peptide complex (specimen) dispersed in an aqueous solvent placed between neodymium magnets and FIG. 44g shows the AFM images of the peptide complex of Example 99 (C-α₅-PEG₁₀) in the presence (right) or absence (left) of a magnetic field magnetic field. FIG. 44h shows the AFM images of the peptide complex of Example 51 (α₁₀-(RGD)₃) in the presence (right) or absence (left) of a magnetic field. FIG. 44i schematically shows a model of a self-assembly processes in the presence (with) or absence (without) of a magnetic field.

It was confirmed that the peptide complex according to the present disclosure is highly sensitive to a magnetic field and the α-helix structure can be controlled with a magnetic field. It was also investigated whether the self-assembly process of the peptide complex is affected by a magnetic field. The peptide complexes of Examples 51 and 99 were dissolved in aqueous solvents to induce self-assembly. The aqueous solvents were sonicated to prevent aggregation for clear observation. The aqueous solvent was placed in the equipment shown in FIG. 44f and measured by AFM after incubation overnight. The AFM measurement was also made before the treatment for comparison before and after the application of the magnetic field.

As shown in FIG. 44g, the peptide complex of Example 99 (C-α₅-PEG₁₀) formed vesicle-like spherical self-assembled structures in the absence of the magnetic field. However, when the magnetic field (0.25 T) was applied, cruciform anisotropic nanostructures, which are unlikely to be found in typical molecular assemblies, were formed.

Referring to FIG. 44h, the peptide complex of Example 47 (L-α₅-PEG₁₀) formed highly uniform micelle-like spherical self-assembled structures in the absence of the magnetic field, but formed anisotropic and unique planar nanostructures when the magnetic field (0.07 T) was applied.

It was confirmed that the self-assembly behavior of the peptide complexes according to the present disclosure is changed by a very weak magnetic field of 0.07-0.25 T. It is because of the restriction in the motional degree of freedom of the peptide complex with the rod-coil during the self-assembly process due to the sensitivity of the MNP helix structure in the peptide complex to the magnetic field. Considering that molecules with the ¹H-¹⁵N RDC values of about 20 kHz are considered to be fully oriented under a magnetic field, only a small fraction of the peptide complex of the present disclosure should be aligned in solution considering the experimental RDC value of α₁₀ (see Table 3). However, the entropic penalty associated with self-assembly should be smaller for the aligned rod-coils than for the rod-coils randomly tumbling in solution (FIG. 44i). Thus, the aligned rod-coils will participate in the self-assembly process more readily than the others, which would alter the overall dynamics of supramolecular crystal growth. Accordingly, since the peptide complex according to the present disclosure includes a hydrophobic peptide with an MNP-helix structure, it has an aligned rod-coil structure when a magnetic field is applied. The peptide complex according to the present disclosure responds sensitively to a magnetic field and its α-helix structure and self-assembled structure are controlled thereby.

### Test Example 6. Analysis of Structural Change of Nanostructure of Peptide Complex and Genetic Material

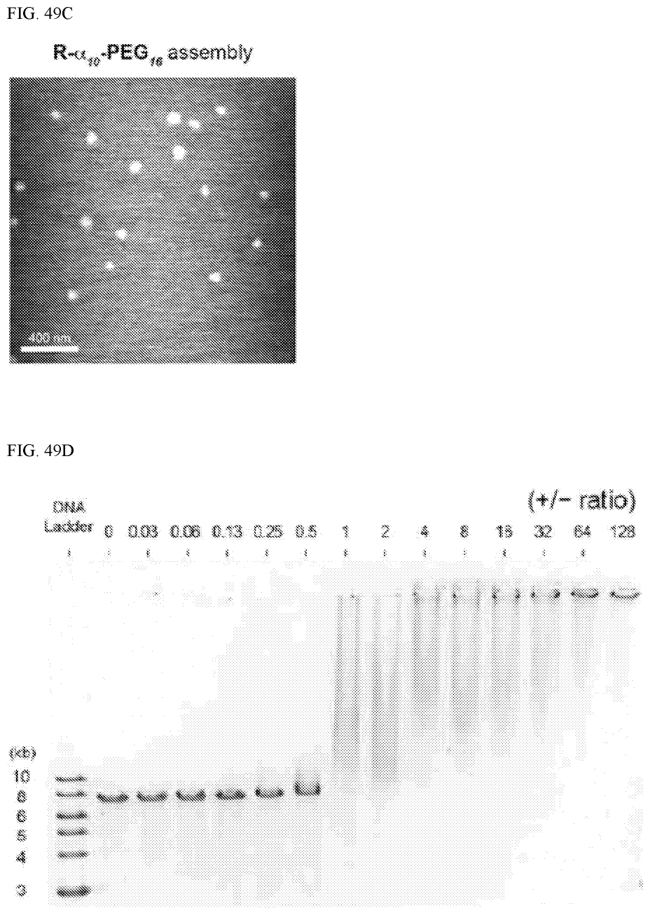

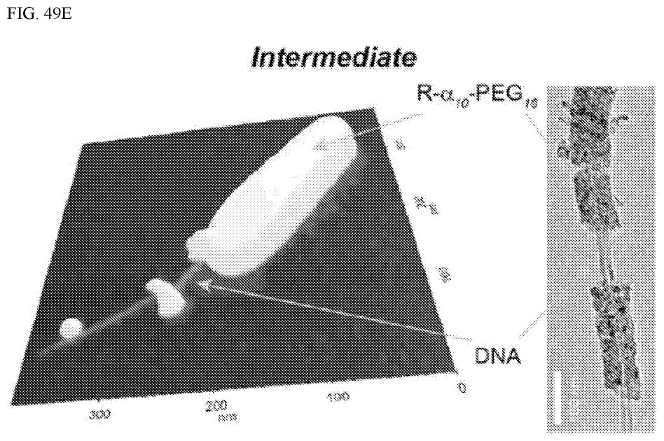

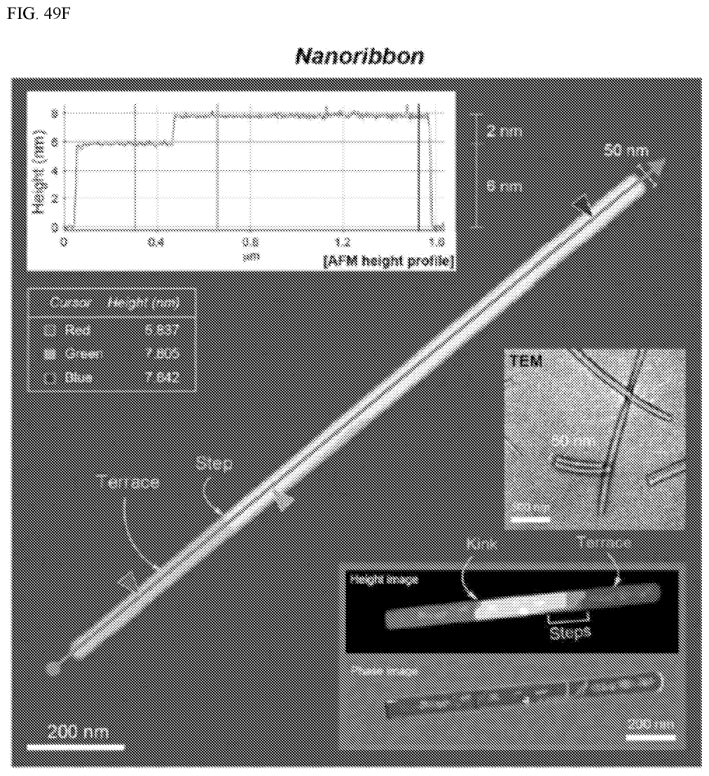

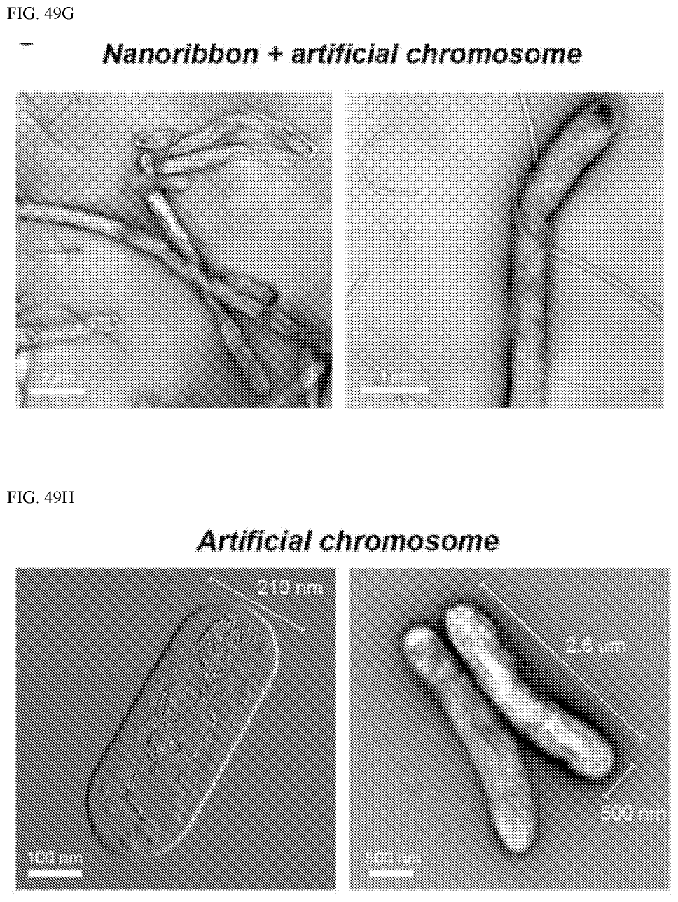

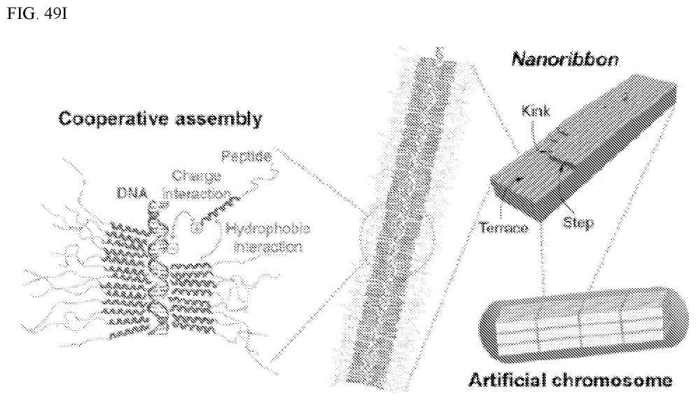

It was investigated whether a complex can be formed through the interaction of the peptide complex according to the present disclosure and a negatively charged genetic material by applying a magnetic field. For this, the peptide complex of Example 60 (R-α₁₀-PEG₁₆) wherein a positively charged arginine (Arg or R) residue is added to the hydrophobic peptide and a linear plasmid DNA were prepared (FIG. 49a).

### 1) Preparation of Linear DNA

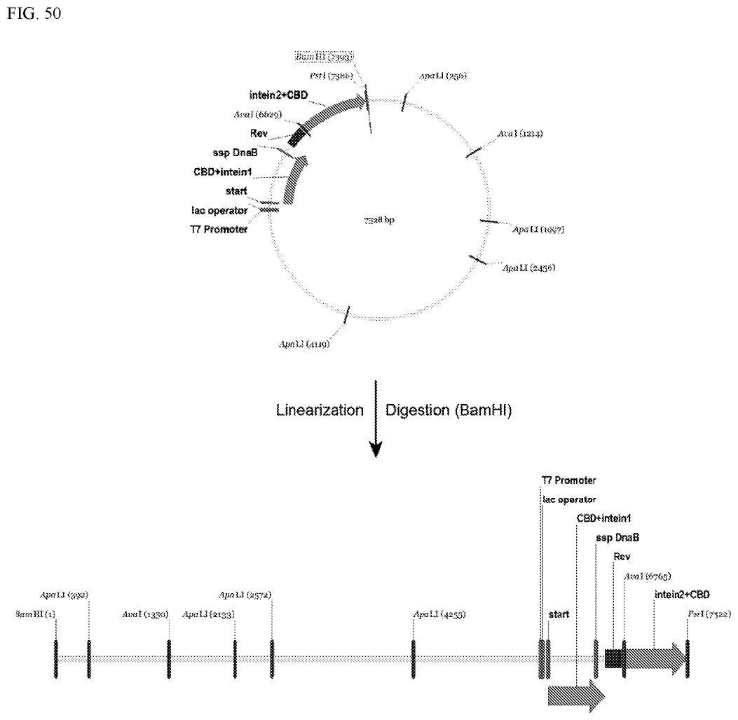

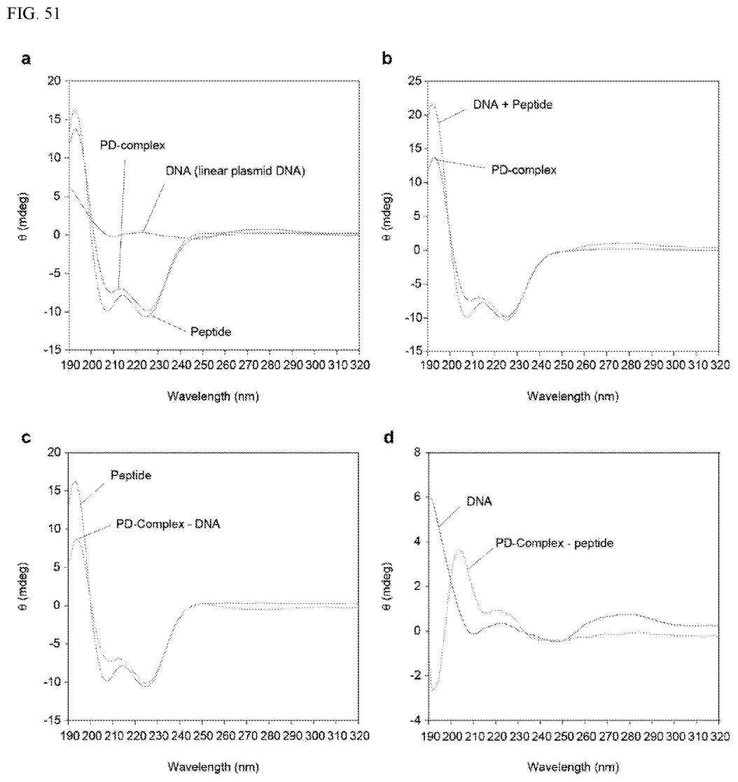

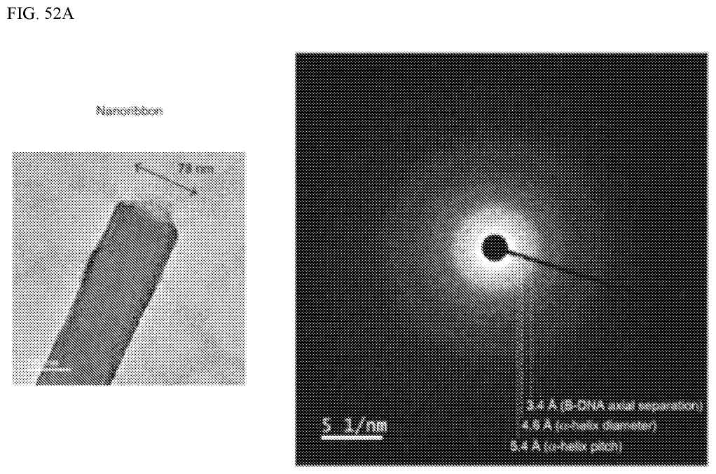

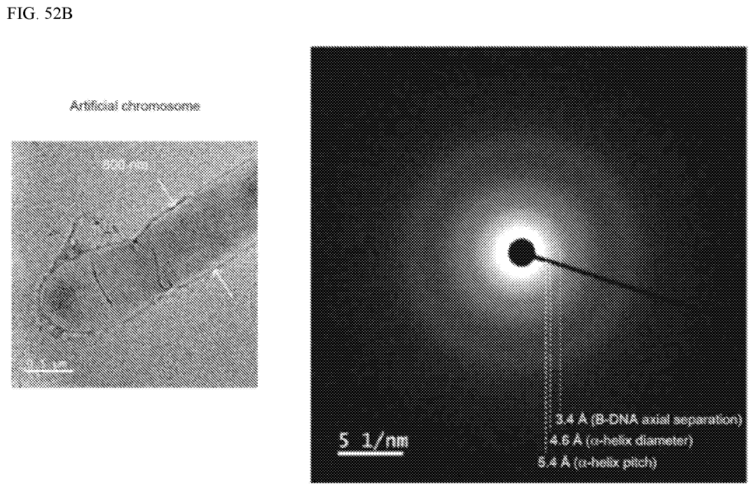

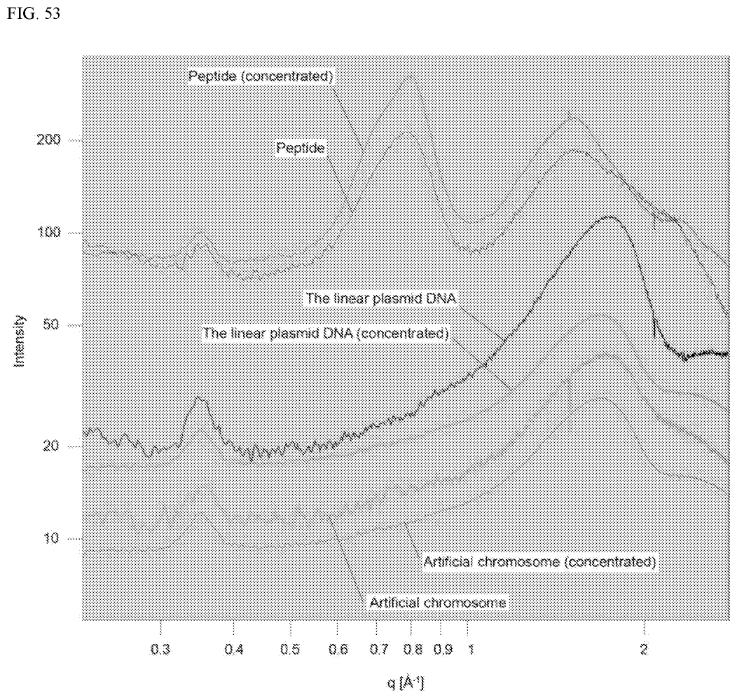

A linearized plasmid DNA with a sufficient length (estimated length: approximately 2.6 mm) was used for facile structural analysis by AFM and TEM. The cleavage map is shown in FIG. 50. A large-scale plasmid DNA (pTWIN-Rev; 7,528 bp) was prepared using the NucleoBond Xtra Maxi Plus kit (Macherey-Nagel, Germany). The plasmid DNA was digested into linear double-stranded DNA with the BamH1 restriction enzyme (New England Biolabs, USA). The linear plasmid DNA was purified from the restriction digest using 1.1% agarose gel. Electrophoresis was conducted at 120 V for 180 minutes in a 1× TBE buffer. After the electrophoresis, the DNA was stained with the SYBR Safe DNA gel stain (Invitrogen, USA). A slice of the DNA band that corresponds to 7.5-kb linear dsDNA was cut out and the linear plasmid DNA was extracted using the MEGA Quick-Spin™ Plus kit (iNtRON, Korea). The purity of the DNA was found to be high (A₂₆₀/A₂₈₀ ratio was 1.8 and A₂₆₀/A₂₃₀ ratio was 2.0-2.2) as measured with a Nanodrop 1000 spectrophotometer (Thermo Scientific, USA).

### 2) Preparation of Peptide-DNA Nanostructure

The peptide complex of Example 60 (R-α₁₀-PEG₁₆) (34 μM in H₂O) was slowly injected into a linear plasmid DNA of equal volume (30 ng/μL in H₂O). Before the mixing, the peptide complex solution was sonicated to prevent nonspecific aggregation of the peptide complex. The peptide-DNA mixture solution was mixed further by gentle pipetting for 1 hour and incubated at room temperature for a prolonged period. Occasionally, 2 μL of the mixture was sampled and the morphological state was characterized by AFM and TEM.

### 3) EMSA (Electrophoretic Mobility Shift Assay)

EMSA was performed to investigate the mode of interaction between the peptide complex of Example 60 (R-α₁₀-PEG₁₆) and the linear plasmid DNA and their binding ratio. The concentration of the peptide complex was increased from 0 μM to 6214 μM, while the concentration of the linear plasmid DNA was fixed to 30 ng/μL (charge ratios (+/−): 0, 0.03, 0.06, 0.13, 0.25, 0.5, 1, 2, 4, 8, 16, 32, 64 and 128). After incubation at room temperature, 10% glycerol was added to the sample for gel loading. Electrophoresis was performed for 100 minutes at 90 V on 1% agarose gel. The bands were visualized by staining with the SYBR Safe DNA gel stain (Invitrogen, USA).

### 4) AFM (Atomic Force Microscopy) Analysis

AFM was performed using the NX10 system (Park Systems, Korea) in a non-contact mode with PPP-NCHR AFM probes (Nanosensors, Switzerland). 2 μL of the sample solution was cast onto a freshly cleaved mica surface and dried. The data obtained with the SmartScan program (Park Systems, Korea) was analyzed using the XEI program (Park Systems, Korea).

### 5) TEM (Transmission Electron Microscopy) and Electron Diffraction

TEM was performed using a JEM-F200 multi-purpose electron microscope (JEOL, Japan) at 200 kV. 2 μL of the sample solution was loaded on a copper grid (carbon type-B grid or Formvar/silicon monoxide grid, 200 mesh copper grids with a 97 μm hole; Ted Pella, USA). 1 hour later, 2 μL of 1-2% uranyl acetate was added to the dried sample for less than 1 minute and the negative stain solution was wicked off with filter paper. To analyze the internal packing structure of the artificial chromosome, the electron diffraction (ED) pattern was observed in selected areas.

FIG. 49a shows the structure of the peptide complex of Example 60 (R-α₁₀-PEG₁₆). In the peptide complex, the N-terminal of the hydrophobic peptide has two positive charges, one derived from the arginine residue and the other from the N-terminal amine. FIG. 49b shows the AFM image of the linear plasmid DNA (7.5 kb) and FIG. 49c the AFM image of the peptide complex of Example 60 (R-α₁₀-PEG₁₆).

As shown in FIGS. 49b and 49c, the linear plasmid DNA was a hair-shaped flexible molecule. It was confirmed that the peptide complex according to Example 60 (R-α₁₀-PEG₁₆) formed a micelle-like spherical self-assembled structure through self-assembly. The peptide complex according to Example 60 (R-α₁₀-PEG₁₆) formed irregular spherical particles due to electrostatic repulsion of positive charges in the micelle core.

FIG. 49d shows the EMSA (electrophoretic mobility shift assay) result for the peptide-DNA nanostructure prepared by mixing the linear plasmid DNA (7.5 kb) with the peptide complex of Example 60 (R-α₁₀-PEG₁₆) at different concentrations.

As shown in FIG. 49d, gradual band smearing occurred in EMSA as the charge ratio (+/−) was increased. This suggests the formation of the peptide-DNA nanostructure. The band smearing that occurred at a charge ratio (+/−) of 1 indicates that the peptide-DNA nanostructure can be formed when the charge ratio (+:−) of the linear plasmid DNA (7.5 kb) and the peptide complex of Example 60 (R-α₁₀-PEG₁₆) is between 0.5:1 and 1:1.

Through the EMSA, it was confirmed that the peptide-DNA nanostructure is formed when the charge ratio (+:−) of the peptide complex of Example 60 (R-α₁₀-PEG₁₆) and the linear plasmid DNA is between 0.5:1 and 1:1. A peptide-DNA nanostructure was prepared by mixing the peptide complex of Example 60 (R-α₁₀-PEG₁₆) and the linear plasmid DNA to have a charge ratio (+/− ratio) of 0.7, i.e., by mixing them at a charge ratio (+:−) of 0.5:1. The mixture was sampled 1 hour, 24 hours and 14 days later and then imaged by AFM and TEM.

FIGS. 49e to 49h show a process of forming the peptide-DNA nanostructure by mixing the linear plasmid DNA (7.5 kb) with the peptide complex of Example 60 (R-α₁₀-PEG₁₆) and a result of monitoring the morphology transformation of the nanostructure by AFM and TEM. FIG. 49e shows the AFM image of the peptide-DNA nanostructure in the early stage, FIG. 49f shows the AFM image of the peptide-DNA nanostructure in the intermediate stage, FIG. 49g shows the TEM image of the peptide-DNA nanostructure in the intermediate-to-late stage, FIG. 49h shows the TEM image of the peptide-DNA nanostructure in the late stage and FIG. 49i shows the mechanism whereby the peptide-DNA nanostructure is formed into a nanoribbon structure.

As shown in FIGS. 49e to 49h, the peptide complex of Example 60 (R-α₁₀-PEG₁₆) and the linear plasmid DNA formed the nanostructure very slowly in a stepwise manner.

First, it was confirmed that the PD complex of the peptide complex of Example 60 (R-α₁₀-PEG₁₆) and the linear plasmid DNA successfully forms a nanostructure even at a charge ratio as low as 0.7.

From FIG. 49e, it was confirmed that the peptide complex of Example 60 (R-α₁₀-PEG₁₆) surrounds the linear plasmid DNA and forms a nanowire-shaped intermediate complex. From FIG. 49f, it can be seen that a rigid ribbon-like structure (nanoribbon) began to appear one day after the mixing of the peptide complex of Example 60 (R-α₁₀-PEG₁₆) and the linear plasmid DNA.

As described above, the peptide complex according to the present disclosure self-assembles cooperatively with the genetic material, during which sequential assembly allows the formation of ordered nanostructures. Flat layers (terraces) and kinks were observed in the nanoribbons, indicating that they are formed by the epitaxial growth mechanism of crystal growth. The presence of steps and kinks between the flat layers (terraces) suggests that the nanoribbon growth can be described by the TSK (terrace-step-kink) model of crystal surface formation. The heights of the steps coincided with the diameter of DNA (2 nm) and its multiples. Thus, it was confirmed that the peptide-DNA nanostructure consists of multiple layers.

The peptide-DNA nanostructure in nanoribbon form according to the present disclosure has a living character. In polymer chemistry, the term “living” refers to the ability of an already formed polymer chain to further react with a freshly supplied monomer. Living behavior has been widely reported during the polymer synthesis such as living polymerization and living supramolecular polymerization. Recently, living behavior has also been found to occur in CDSA (living crystallization-driven self-assembly) or fibrilization of amyloid-β peptides. The discovery in the present disclosure is remarkable in that it presents a new structure.

The peptide-DNA nanostructure formed by mixing of the peptide complex of Example 60 (R-α₁₀-PEG₁₆) and the linear plasmid DNA has a living nanoribbon structure.

The peptide-DNA nanostructure having a living nanoribbon structure has been finally (2 weeks after incubation) confirmed as a nanostructure self-assembled into micrometer-scale objects, similar to human chromosomes in structure. The structure was termed ‘artificial chromosome’ structure. The ‘artificial chromosome’ structure of the peptide-DNA nanostructure was final and maintained for a long time.

FIG. 50 shows the cleavage map of the plasmid DNA used in the present disclosure, which was cleaved at the Bam HI site. FIG. 51 shows the CD spectra of the peptide-DNA nanostructure of the peptide complex of Example 60 and a linear plasmid DNA and FIG. 52A shows the SAED (selected area electron diffraction) pattern of the peptide-DNA nanostructure in nanoribbon state and FIG. 52B shows the peptide-DNA nanostructure in artificial chromosome state. FIG. 53 shows the WAXS (synchrotron wide-angle X-ray scattering) analysis result of the peptide-DNA nanostructure of the peptide complex of Example 60 and a linear plasmid DNA.

As shown in FIG. 51, FIG. 52A, FIG. 52B and FIG. 53, the peptide-DNA nanostructure (PD complex) maintained the B-DNA structure, DNA double helix structure and α-helix structure in both the peptide complex of Example 60 (R-α₁₀-PEG₁₆) and the linear plasmid DNA.

Through these results, it was confirmed that a nanostructure with an artificial chromosome-like structure is prepared through the following steps when the peptide complex of Example 60 (R-α₁₀-PEG₁₆) is mixed with the linear plasmid DNA: when the peptide complex of Example 60 (R-α₁₀-PEG₁₆) is mixed with the linear plasmid DNA, a nanostructure with a living nanoribbon structure is formed as the peptide complex surrounds the linear plasmid DNA and a nanostructure with an artificial chromosome structure is formed finally as the structure is stabilized gradually.

It is formed through charge interaction between the arginine residue (positively charged amino acid) in the peptide complex of Example 60 (R-α₁₀-PEG₁₆) and DNA and cooperative hydrophobic interaction between MNP-helices. The finally formed nanostructure in artificial chromosome form consists of a crystalline core and a solvated corona with a width of 200-500 nm and a length of 1-3 μm.

### Test Example 7. Analysis of Stability of Nanostructure in Artificial Chromosome Form

### 1) Preparation of Linear DNA

A linearized plasmid DNA with a sufficient length (estimated length: approximately 2.6 mm) was used for facile structural analysis by AFM and TEM. The cleavage map is shown in FIG. 50. A large-scale plasmid DNA (pTWIN-Rev; 7,528 bp) was prepared using the NucleoBond Xtra Maxi Plus kit (Macherey-Nagel, Germany). The plasmid DNA was digested into linear double-stranded DNA with the BamH1 restriction enzyme (New England Biolabs, USA). The linear plasmid DNA was purified from the restriction digest using 1.1% agarose gel. Electrophoresis was conducted at 120 V for 180 minutes in a 1× TBE buffer. After the electrophoresis, the DNA was stained with the SYBR Safe DNA gel stain (Invitrogen, USA). A slice of the DNA band that corresponds to 7.5-kb linear dsDNA was cut out and the linear plasmid DNA was extracted using the MEGA Quick-Spin™ Plus kit (iNtRON, Korea). The purity of the DNA was found to be high (A₂₆₀/A₂₈₀ ratio was 1.8 and A₂₆₀/A₂₃₀ ratio was 2.0-2.2) as measured with a Nanodrop 1000 spectrophotometer (Thermo Scientific, USA).

### 2) Preparation of Peptide-DNA Nanostructure

The peptide complex of Example 60 (R-α₁₀-PEG₁₆) (34 μM in H₂O) was slowly injected into a linear plasmid DNA of equal volume (30 ng/μL in H₂O). Before the mixing, the peptide complex solution was sonicated to prevent nonspecific aggregation of the peptide complex. The peptide-DNA mixture solution was mixed further by gentle pipetting for 1 hour and incubated at room temperature for 14 days to prepare a peptide-DNA nanostructure (artificial chromosome form).

### 3) DNase I Protection Assay

A sample (the linear plasmid DNA or the artificial chromosome) containing 60 ng (2 μL; 30 ng/μL) of DNA was mixed with 0.1 μL (0.2 unit) of DNase I and 1 μL of a 10× DNase I reaction buffer. The volume of the mixture was 10 μL. After 20 minutes of incubation at 37° C., 0.3 μL of 0.05 M EDTA was added to inactivate the enzyme. Then, the sample was mixed with 1.7 μL of 60% glycerol and electrophoresed on 1.1% agarose gel (120 minutes, 100 V). For visualization, the DNA was stained with an SYBR Safe DNA gel stain (Invitrogen, USA). To evaluate the time-dependent kinetics of DNA degradation, UV absorbance at 260 nm was recorded over time after the addition of DNase I. Each sample (71.2 μL) containing DNA (30 ng/μL) and a 10× DNase I reaction buffer (8 μL) was mixed in a cuvette. Then, DNase I (0.8 μL) was added to the cuvette and the sample was mixed by pipetting for about 90-105 seconds. UV absorbance at 260 nm (A₂₆₀) was measured using a V-650 UV-vis spectrophotometer (JASCO, Japan). A quartz cuvette with a path length of 10 mm was used. The absorbance was recorded at intervals of 10 seconds.

4) Information Recovery from Artificial Chromosome

To assess the capability of recovering DNA information stored in the artificial chromosome, PCR (polymerase chain reaction) was conducted to amplify a specific DNA fragment of the sample DNA. The specific 310-bp fragment was amplified using the sense primer 5′-ACG GAG ACT GGA GTC GAA GAG G-3′ (SEQ ID NO 106) and the antisense primer 5′-GTA GGG CAA CTA GTG CAT CTC CC-3′ (SEQ ID NO 107). Each reaction mixture (50 μL) contained 50 ng of DNA, 10 pmol of each primer and 25 μL of 2× Quick Taq HS DyeMix (Toyobo, Japan). PCR amplification (30 cycle) was performed using a T100 thermal cycler (Bio-Rad, USA). After the amplification, the product was electrophoresed for 120 minutes at 80 V on 2% agarose gel (TBE system). A SYBR Safe DNA gel stain (Invitrogen, USA) was used for DNA staining.

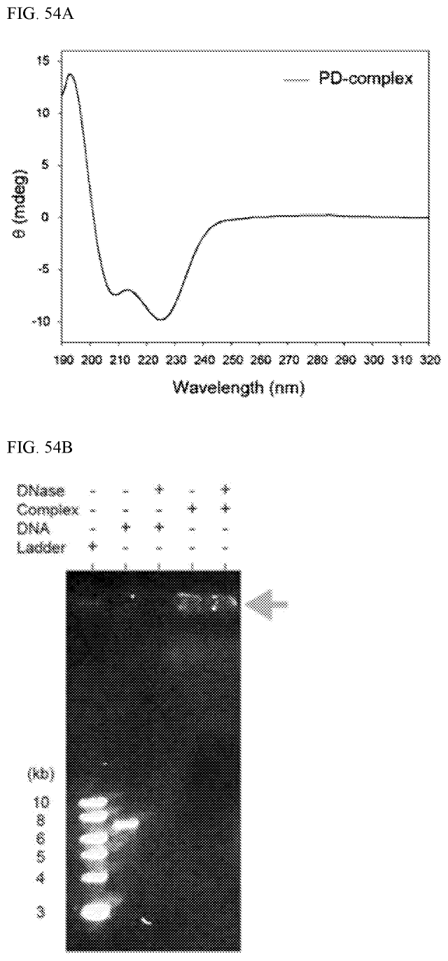

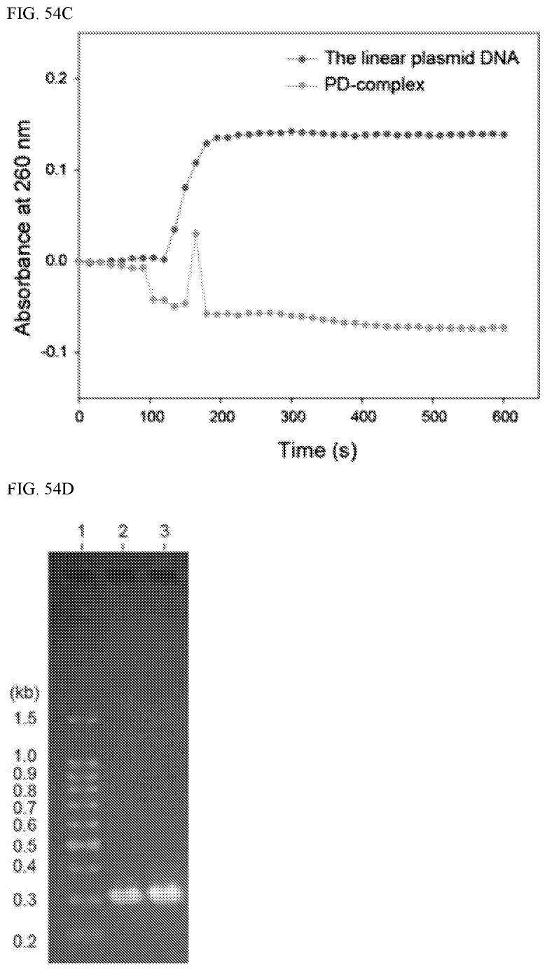

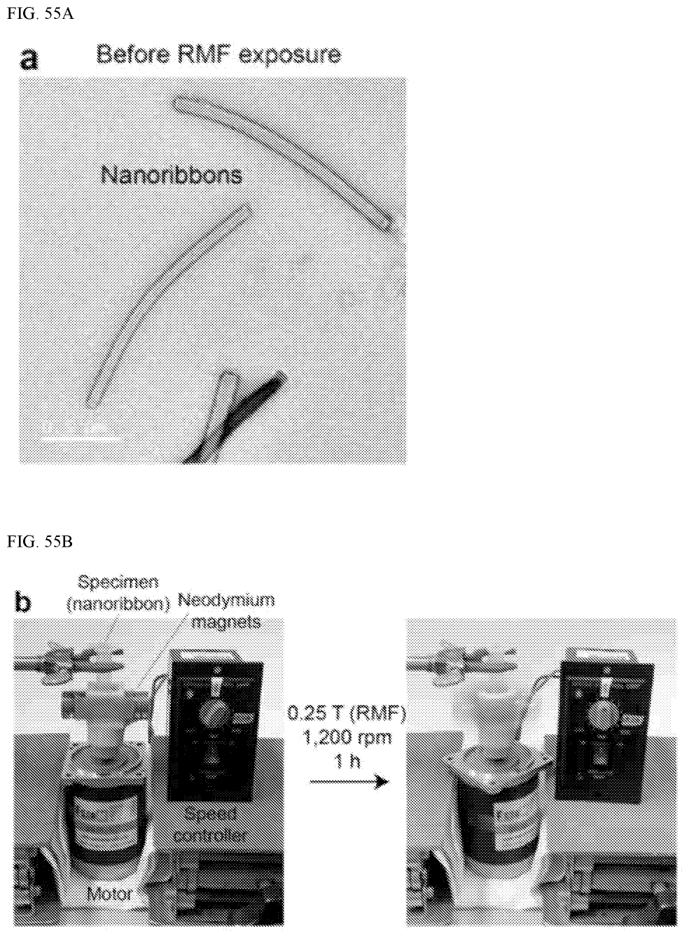

FIG. 54a shows the CD spectrum of the peptide-DNA nanostructure (artificial chromosome form) of the peptide complex of Example 60 and the linear plasmid DNA.

As shown in FIG. 54a, in the peptide-DNA nanostructure (artificial chromosome form), DNA is packaged tightly by the peptide complex of Example 60 (R-α₁₀-PEG₁₆). In human chromosomes, 2 nm DNA in the small cell nucleus (˜6 μm in diameter) is packaged tightly with the help of positively charged histone proteins. Therefore, the peptide-DNA nanostructure (artificial chromosome form) is structurally very similar to human chromosomes.

The peptide-DNA nanostructure (artificial chromosome form) according to the present disclosure is also very similar to human chromosomes (length: about 2-13 μm) in dimension and shape. Due to this structure, the peptide-DNA nanostructure (artificial chromosome form) according to the present disclosure can store a large quantity of biological data of genetic materials in minimal space. It can stably store the biological data without loss and the information of the genetic material can be retrieved through general analysis methods such as PCR, etc. Since the peptide-DNA nanostructure (artificial chromosome form) according to the present disclosure can store DNA information at high density, it can be utilized as a new DNA data storage medium.

FIG. 54b shows the electrophoresis result of the peptide-DNA nanostructure (artificial chromosome form) in the presence of DNase 1. Ladder indicates a DNA size marker, DNA indicates the linear plasmid DNA, Complex indicates the peptide-DNA nanostructure (artificial chromosome form) and DNase indicates DNase 1. The arrow indicates the peptide-DNA nanostructures (artificial chromosome form) trapped in the lanes 4 and 5.

FIG. 54c shows the result of adding the peptide-DNA nanostructure (artificial chromosome form) of the peptide complex of Example 60 and the linear plasmid DNA in the presence of DNase I and analyzing its degradation with time (0-600 seconds) by electrophoresis.

FIG. 54d shows the PCR analysis result of the peptide-DNA nanostructure (artificial chromosome form) of the peptide complex of Example 60 and the linear plasmid DNA. Lane 1 indicates a DNA size marker, lane 2 indicates a PCR-amplified linear plasmid DNA and lane 3 indicates a PCR-amplified DNA from the peptide-DNA nanostructure (artificial chromosome form).

To use the peptide-DNA nanostructure (artificial chromosome form) for the storage of genetic information and biological data, it should be able to protect DNA from enzymatic, chemical and physical damage. Therefore, the peptide-DNA nanostructure (artificial chromosome form) was exposed to DNase I for a long time and it was investigated whether the DNA is maintained without degradation.

From FIG. 54b, it can be seen that the peptide-DNA nanostructure (artificial chromosome form) of the peptide complex of Example 60 according to the present disclosure and the linear plasmid DNA effectively protects DNA from the attack by DNase 1.

As shown in FIG. 54c, it can be seen that the peptide-DNA nanostructure (artificial chromosome form) according to the present disclosure safely stores DNA without damage even after exposure to DNase I for 600 seconds or longer. In contrast, in naked state, the DNA was quickly degraded upon the addition of DNase 1.

As shown in FIG. 54d, the specific fragment of the linear plasmid DNA was amplified successfully when the peptide-DNA nanostructure (artificial chromosome form) according to the present disclosure was subjected to the standard PCR protocol. Through this, it was confirmed that, when mixed with the linear plasmid DNA, the peptide complex of Example 60 (R-α₁₀-PEG₁₆) forms a nanostructure in artificial chromosome form and stably stores genetic materials such as DNA and the stored or delivered genetic materials such as DNA are maintained without damage. In addition, since the peptide complex of Example 60 (R-α₁₀-PEG₁₆) does not interfere with the DNA amplification process by primers, PCR can be performed on the peptide-DNA nanostructure (artificial chromosome form) according to the present disclosure without an additional purification process.

### Test Example 8. Control of Degradation (Disassembly) of Nanostructure in Artificial Chromosome Form with Magnetic Field

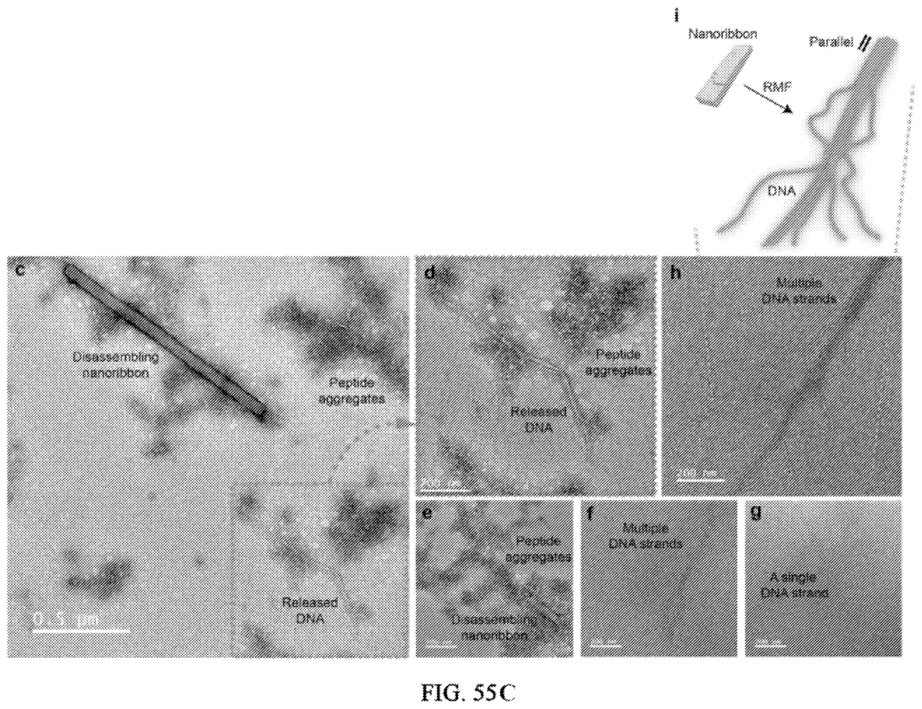

The peptide-DNA nanostructure prepared in Test Example 7 (nanoribbon form) was prepared. The peptide-DNA nanostructure (artificial chromosome form) was placed in the equipment shown in FIG. 55b as a ‘specimen’. The equipment is for analyzing the structural change of the peptide-DNA nanostructure (nanoribbon form) by an RMF (rotating magnetic field) generated by permanent magnets. After exposure to the rotating magnetic field (RMF, 0.25 T, 1200 rpm) for 1 hour, the nanostructure was imaged by AFM and TEM. The AFM and TEM analysis conditions were the same as in Test Examples 6 and 7.

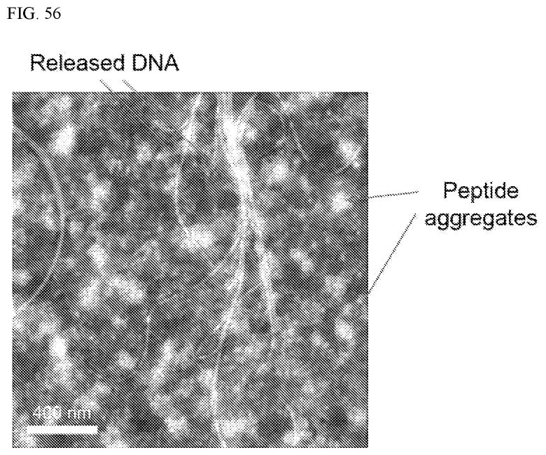

FIG. 55a shows the TEM image of the peptide-DNA nanostructure (nanoribbon form) of the peptide complex of Example 60 and the linear plasmid DNA before exposure to the magnetic field and FIG. 55b shows the experimental equipment for generating an RMF (rotating magnetic field) from permanent magnets. FIG. 55c shows the TEM images of the peptide-DNA nanostructure (nanoribbon form) of the peptide complex of Example 60 and the linear plasmid DNA after exposure to the rotating magnetic field. FIG. 56 shows the AFM image of the peptide-DNA nanostructure (nanoribbon form) of the peptide complex of Example 60 and the linear plasmid DNA after exposure to a static magnetic field (0.1 T) for 2 weeks.

As shown in FIG. 56, the peptide-DNA nanostructure according to the present disclosure (nanoribbon form) was degraded when exposed to the magnetic field. However, 2 weeks or longer was required under a static magnetic field (0.1 T) condition.

As shown in FIGS. 55A to 55C, the peptide-DNA nanostructure according to the present disclosure (nanoribbon form) was degraded within 1 hour when exposed to the rotating magnetic field. From FIGS. 55a to 55c, it was confirmed that DNAs, peptide aggregate sand partially unfolded nanoribbons were released as the peptide-DNA nanostructure according to the present disclosure (nanoribbon form) was degraded. The released DNAs existed as single strands or multiple DNA strands. Several DNA strands lied parallel to each other (as shown in h and i of FIG. 55C).

That is to say, it can be seen that the assembly or disassembly of the peptide-DNA nanostructure according to the present disclosure can be controlled effectively with a magnetic field.

### Test Example 9. Analysis of α-Helix Structure of Hydrophobic Peptides of Examples 1-36

The CD spectra of the hydrophobic peptides of Examples 1-36 were measured and the θ_{222 nm}/θ₂₀₈ₙₘ ratio was analyzed therefrom. The result is shown in Table 10. It can be seen that the helix structure is stabilized when the θ₂₂₂ₙₘ/θ₂₀₈ₙₘ ratio is 1.0-1.2.

TABLE 10

| | θ_{222 nm}/θ_{208 nm} ratio |
|---|---|
| Example 1 | 0.64 |
| Example 2 | 0.72 |
| Example 3 | 0.85 |
| Example 4 | 0.92 |
| Example 5 | 0.99 |
| Example 6 | 1.1 |
| Example 7 | 1.08 |
| Example 8 | 1.13 |
| Example 9 | 1.13 |
| Example 10 | 1.21 |
| Example 11 | 1.19 |
| Example 12 | 1.24 |
| Example 13 | 1.2 |
| Example 14 | 1.26 |
| Example 15 | 1.29 |
| Example 16 | 1.28 |
| Example 17 | 1.21 |
| Example 18 | 1.24 |
| Example 19 | 1.25 |
| Example 20 | 0.63 |
| Example 21 | 0.74 |
| Example 22 | 0.8 |
| Example 23 | 0.88 |
| Example 24 | 0.95 |
| Example 25 | 1.02 |
| Example 26 | 1.08 |
| Example 27 | 1.09 |
| Example 28 | 1.13 |
| Example 29 | 1.08 |
| Example 30 | 1.14 |
| Example 31 | 1.24 |
| Example 32 | 1.3 |
| Example 33 | 1.24 |
| Example 34 | 1.28 |
| Example 35 | 1.32 |
| Example 36 | 1.25 |

As shown in Table 10, it was confirmed that the hydrophobic peptides of Examples 6-19 and 25-36 have a perfect α-helix structure. It can be seen that it is preferred that the repeat unit a constituting the hydrophobic peptide is repeated 6-13 times, most specifically 6-10 times, to achieve the most stable α-helix structure.

### Test Example 10. Purification of Peptide Complexes and Analysis of Molecular Weight

The peptide complexes of Examples 37-40, 46-53, 60-63, 66-67, 71-72 and 90-91 were purified by HPLC and analyzed by MALDI-TOF spectroscopy. The HPLC condition was as follows: C4 semiprep TFA ACN 15-35% 10-60 min, mobile phase: ACN (TFA 0.1%), gradient: 15-35%/10-60 min (+0.4%/min), flow rate: 2.000 mL/min, injection volume: 4.5 mL, injection solvent: ACN 5%, temperature: 25° C., UV detection: 230 nm.

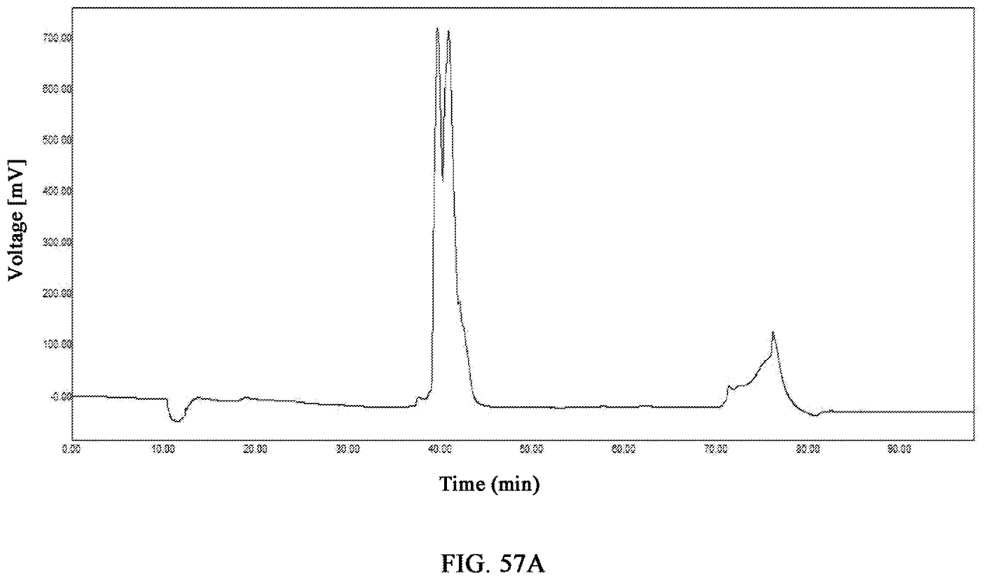

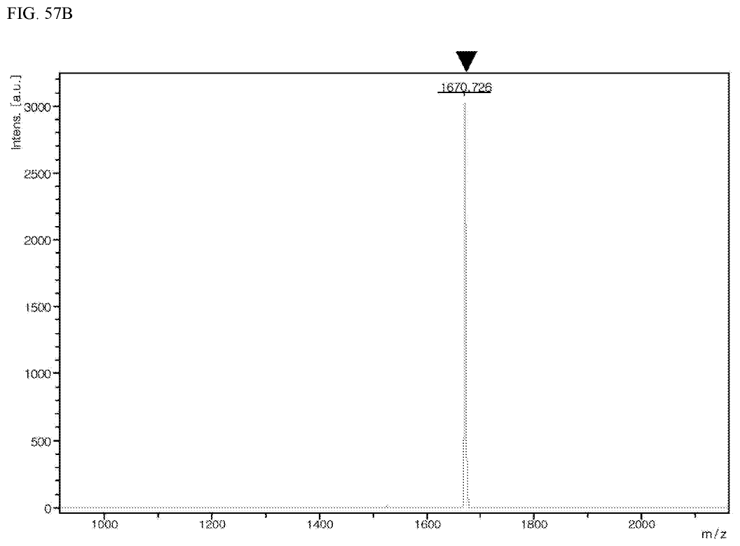

FIG. 57A shows the HPLC analysis result of the peptide complex of Example 37 (1.286 mM) and FIG. 57B shows the MALDI-TOF mass spectrometry analysis result of the peptide complex of Example 37 (1.286 mM). As shown in FIGS. 57A and 571B, the peptide complex of Example 37 had a molecular weight of 1670.78 g/mol and a purity >95%.

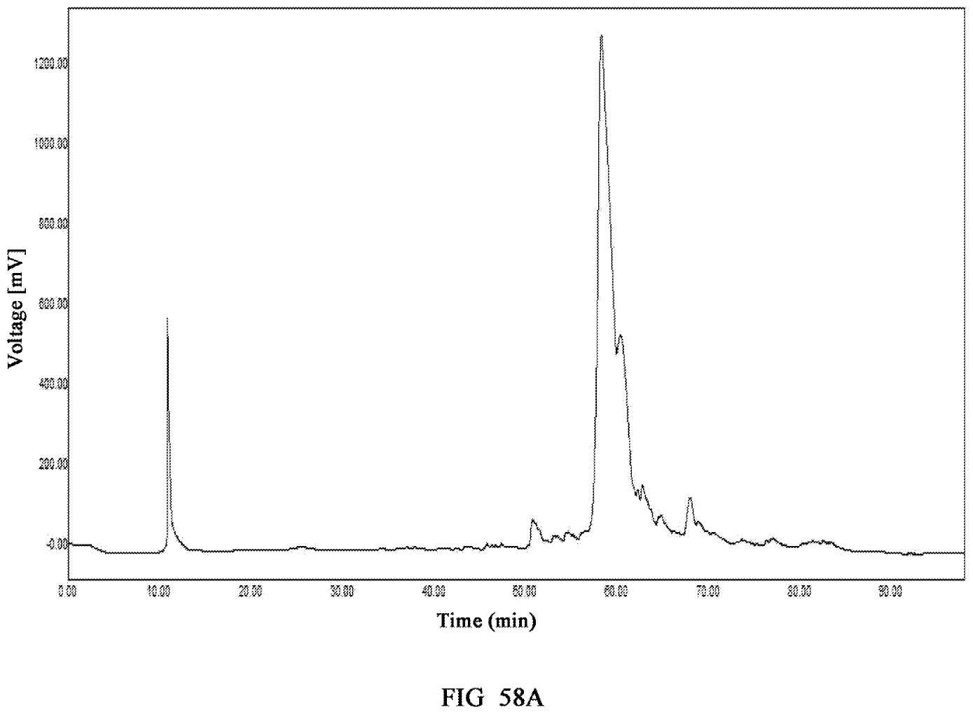

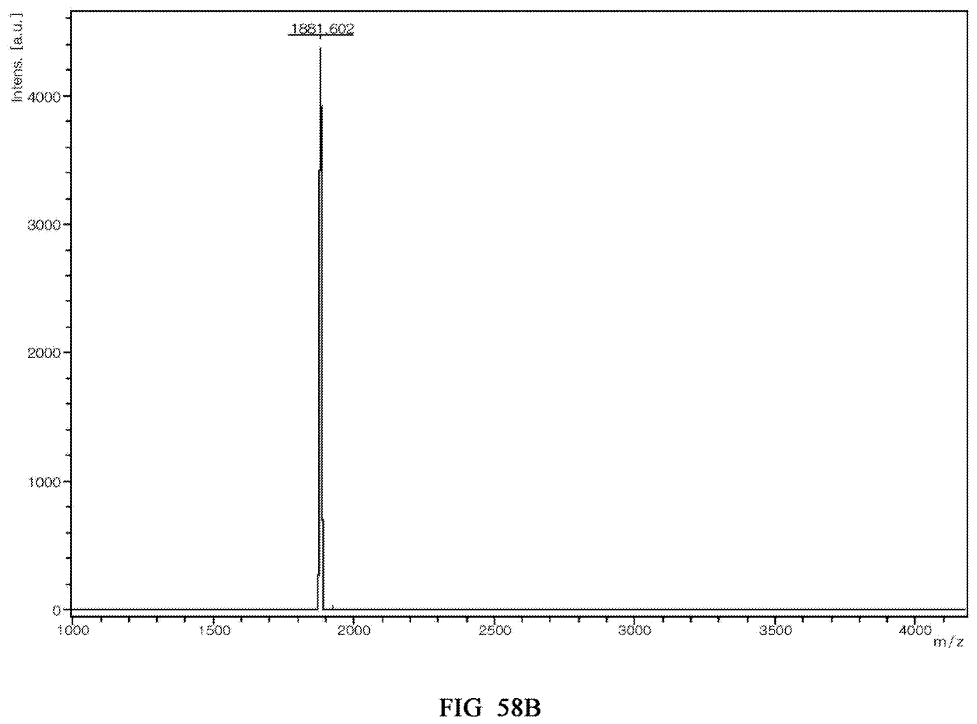

FIG. 58A shows the HPLC analysis result of the peptide complex of Example 38 and FIG. 58B shows the MALDI-TOF mass spectrometry analysis result of the peptide complex of Example 38. It was confirmed that the peptide complex of Example 38 was synthesized successfully with a purity >95%.

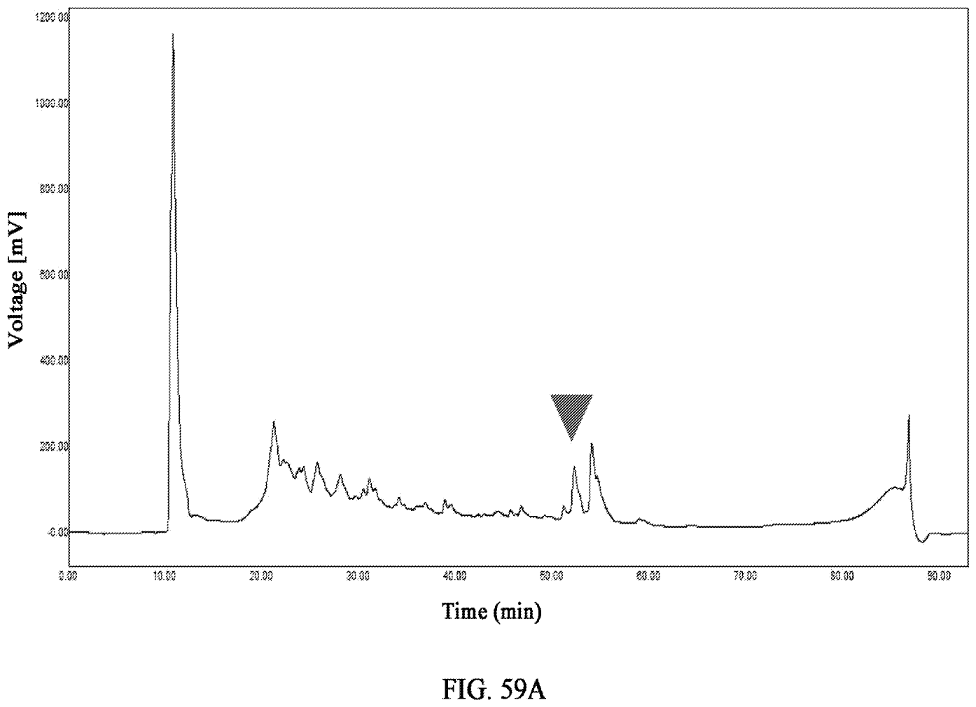

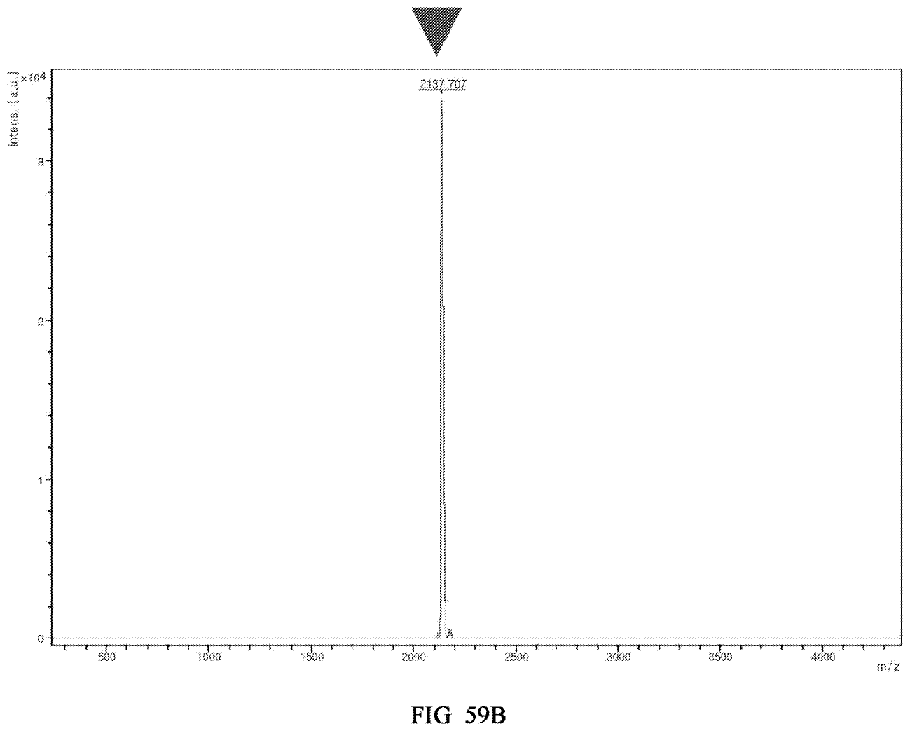

FIG. 59A shows the HPLC analysis result of the peptide complex of Example 39 and FIG. 59B shows the MALDI-TOF mass spectrometry analysis result of the peptide complex of Example 39. The peptide complex of Example 39 was synthesized successfully with a purity >95%. The molecular weight of the peptide complex of Example 39 was: molecular weight calc.=2139.33 g/mol and molecular weight mes.=2137.71 g/mol.

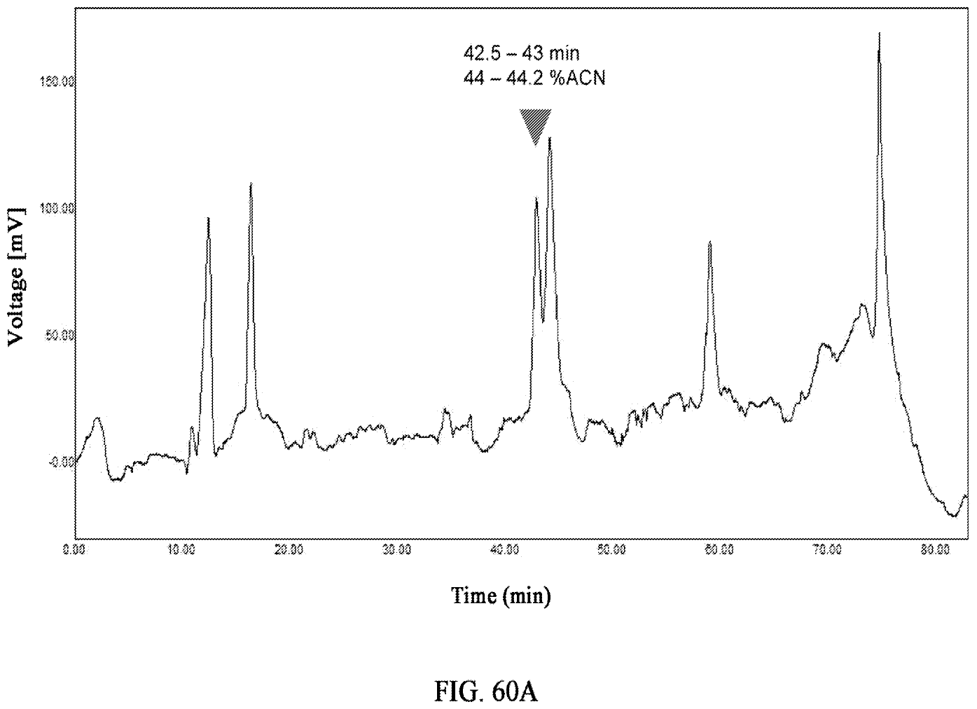

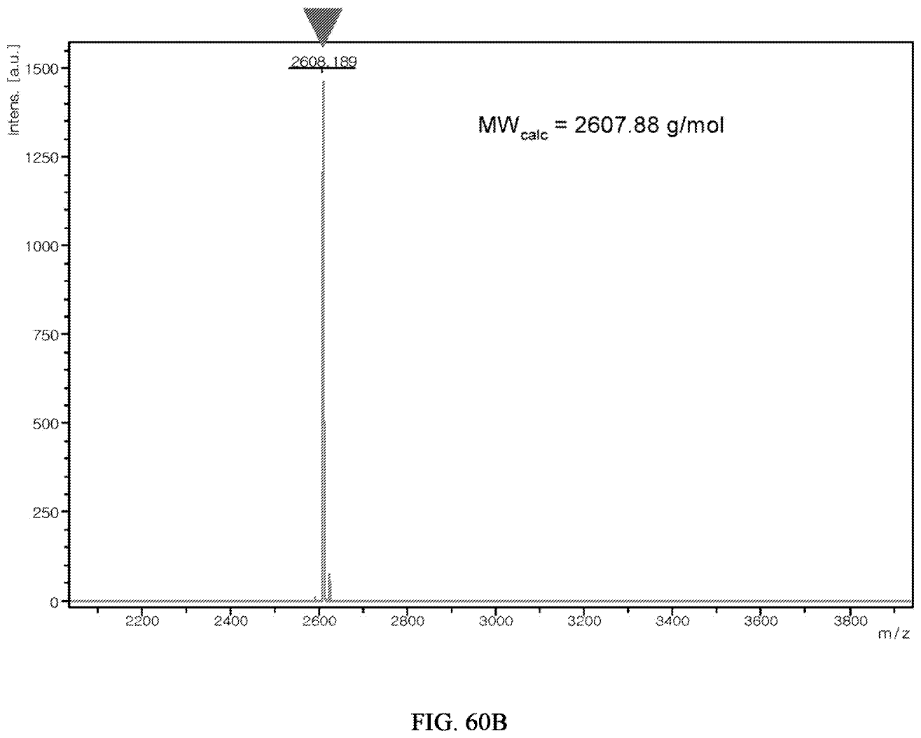

FIG. 60A shows the HPLC analysis result of the peptide complex of Example 40 and FIG. 60B shows the MALDI-TOF mass spectrometry analysis result of the peptide complex of Example 40. The peptide complex of Example 40 was synthesized successfully with a purity >95%. The molecular weight of the peptide complex of Example 40 was: molecular weight calc.=2607.88 g/mol.

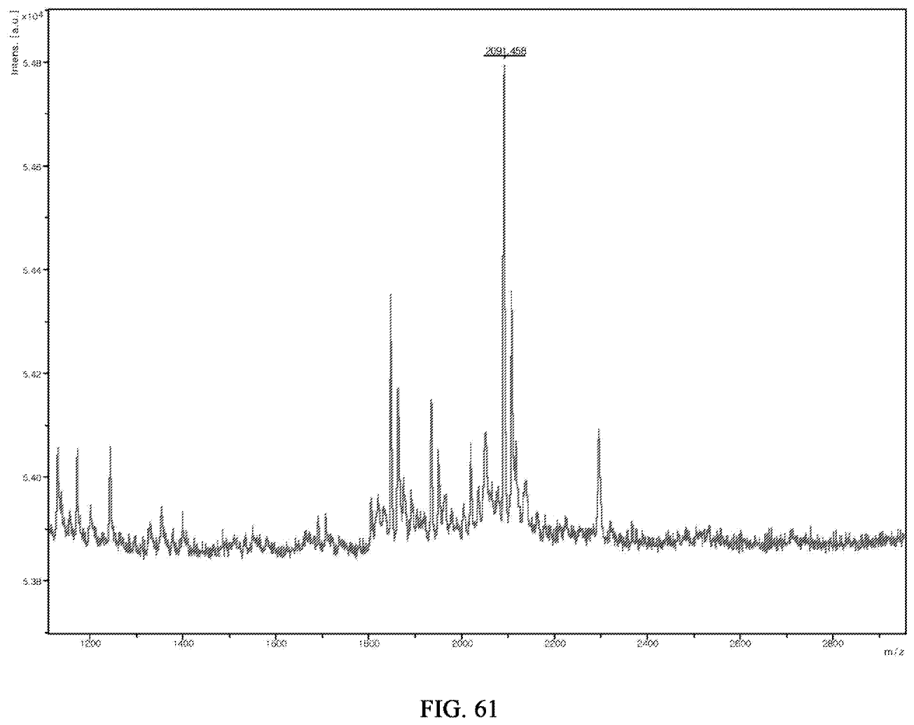

FIG. 61 shows the HPLC analysis result of the peptide complex of Example 53. It can be seen that the peptide complex of Example 53 was synthesized successfully.

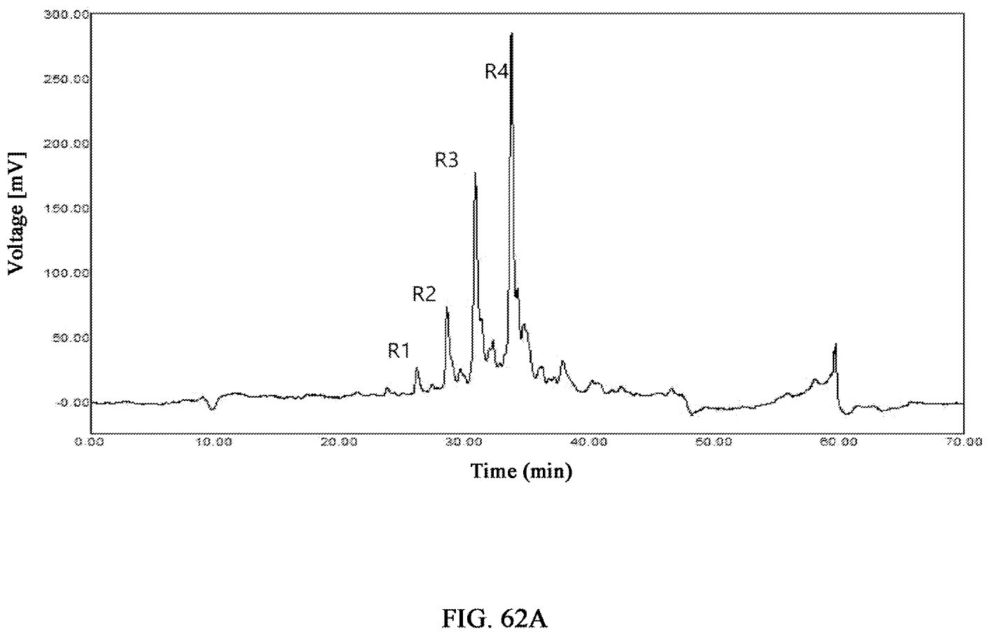

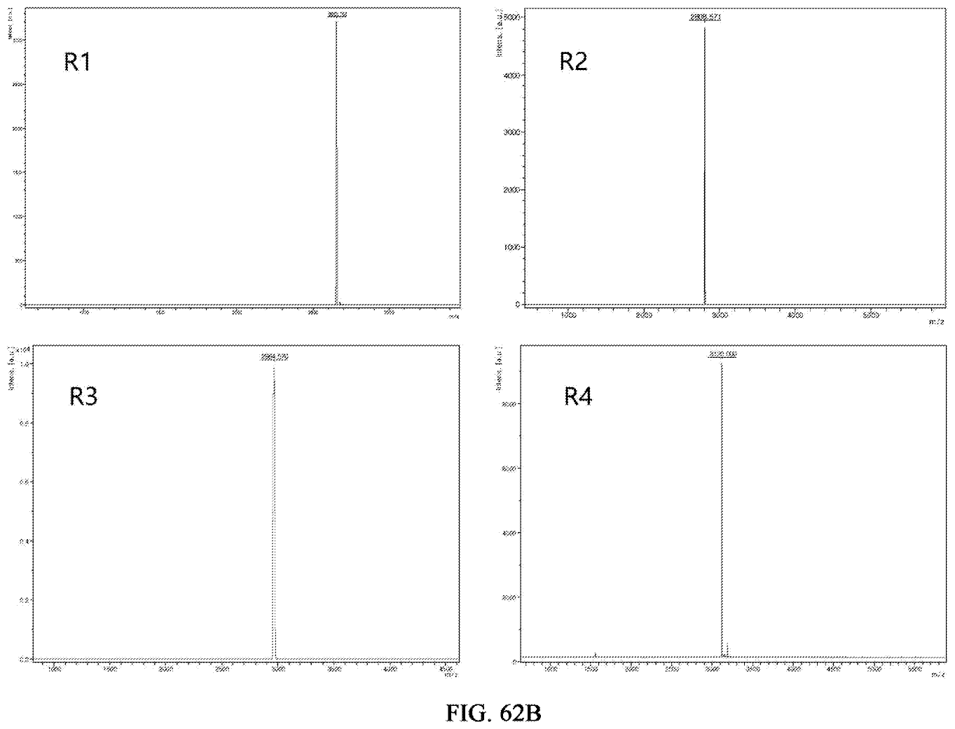

FIG. 62A shows the HPLC analysis result of the peptide complexes of Examples 60-63 and FIG. 62B shows the MALDI-TOF mass spectrometry analysis result of the peptide complexes of Examples 60-63. In the graphs, R1 indicates the peptide complex of Example 60, R2 indicates the peptide complex of Example 61, R3 indicates the peptide complex of Example 62 and R4 indicates the peptide complex of Example 63.

As seen from FIGS. 62A and 62B, the peptide complexes of Examples 60-63 were synthesized successfully with a purity >95%. The molecular weight of the peptide complexes of Examples 60-63 was 2653.15 g/mol, 2809.34 g/mol, 2965.53 g/mol and 3121.72 g/mol, respectively.

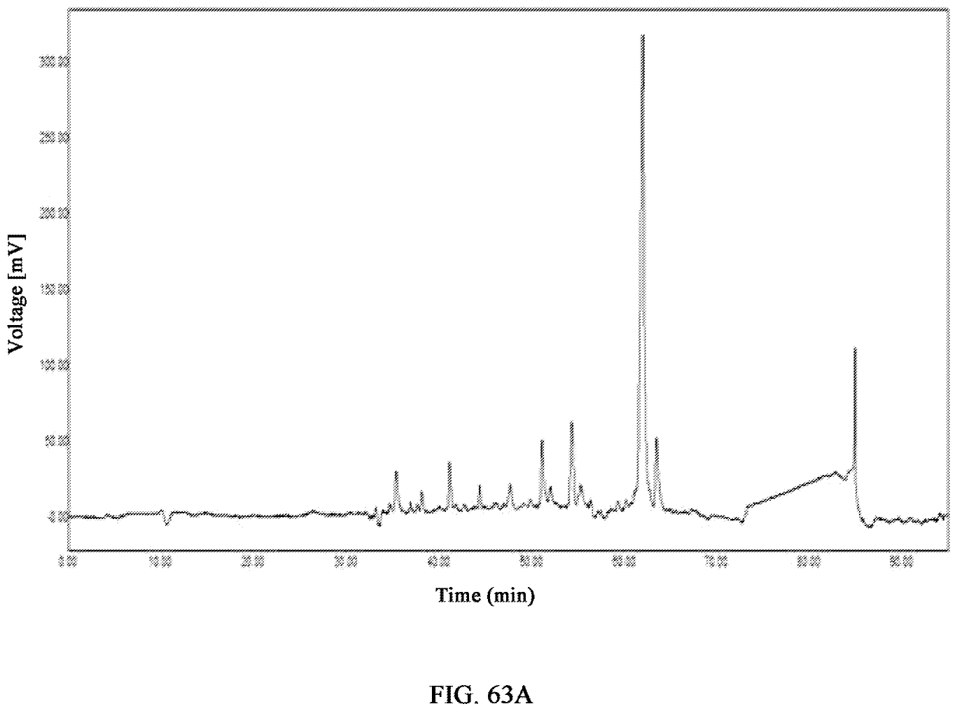

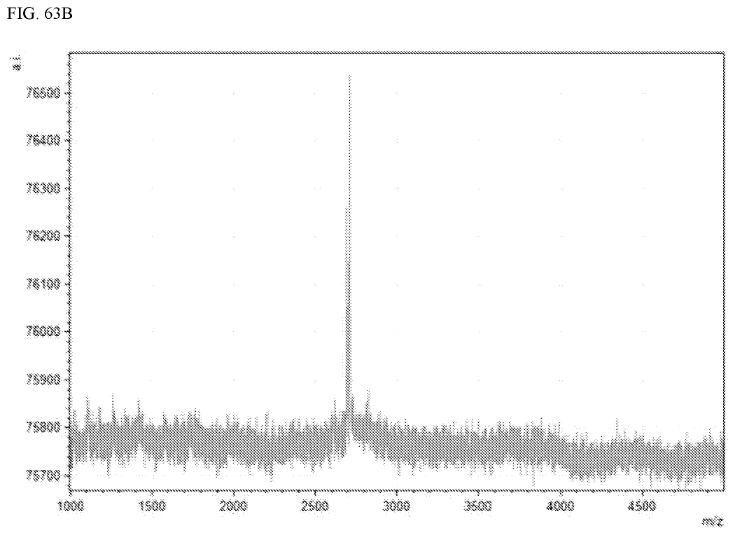

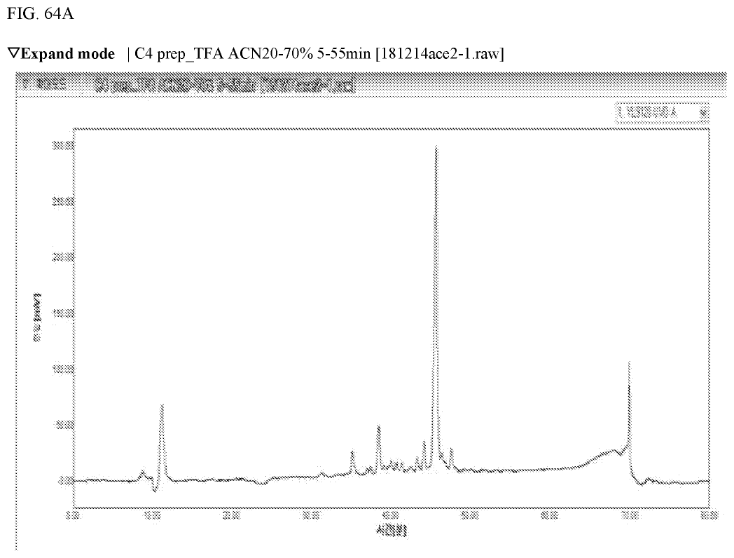

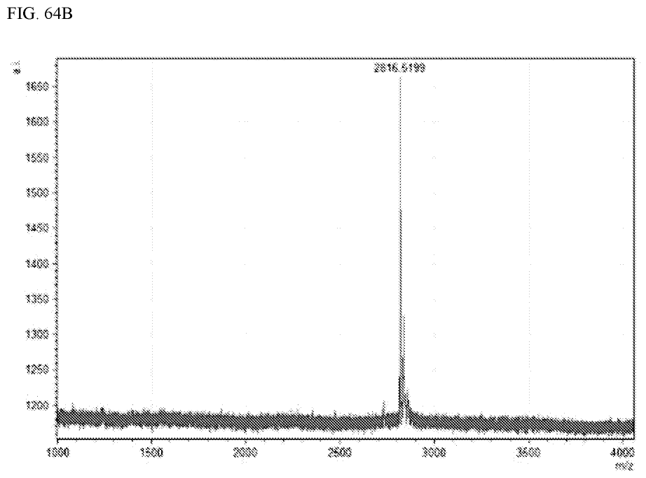

FIG. 63A shows the HPLC analysis result of the peptide complex of Example 90 and FIG. 63B shows the MALDI-TOF mass spectrometry analysis result of the peptide complex of Example 90. It was confirmed that the peptide complex of Example 90 was synthesized successfully with a purity >95%.

FIG. 64A shows the HPLC analysis result of the peptide complex of Example 91 and FIG. 64B shows the MALDI-TOF mass spectrometry analysis result of the peptide complex of Example 91. It was confirmed that the peptide complex of Example 91 was synthesized successfully with a purity >95%.

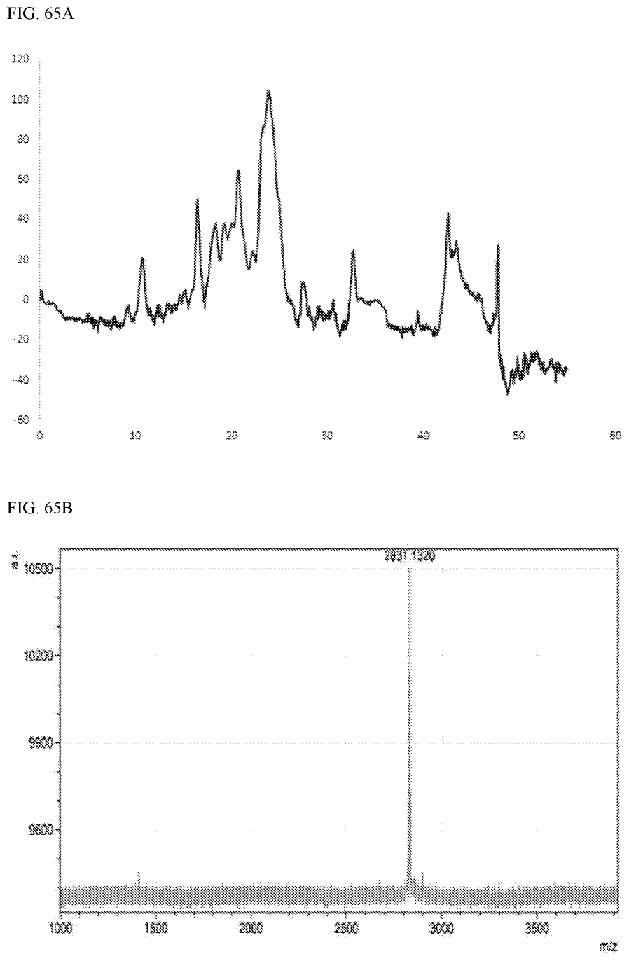

FIG. 65A shows the HPLC analysis result of the peptide complex of Example 71 and FIG. 65B shows the MALDI-TOF mass spectrometry analysis result of the peptide complex of Example 71. It was confirmed that the peptide complex of Example 71 was synthesized successfully with a purity >95%. The molecular weight of the peptide complex of Example 71 was 2833.17 g/mol.

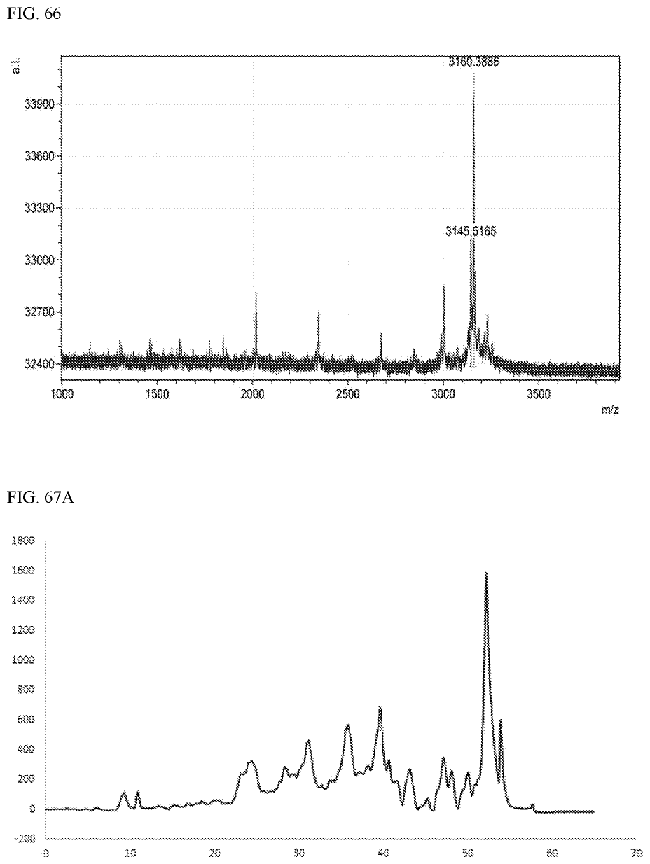

FIG. 66 shows the MALDI-TOF mass spectrometry analysis result of the peptide complex of Example 72. It was confirmed that the peptide complex of Example 72 was synthesized successfully and the molecular weight was 2833.17 g/mol.

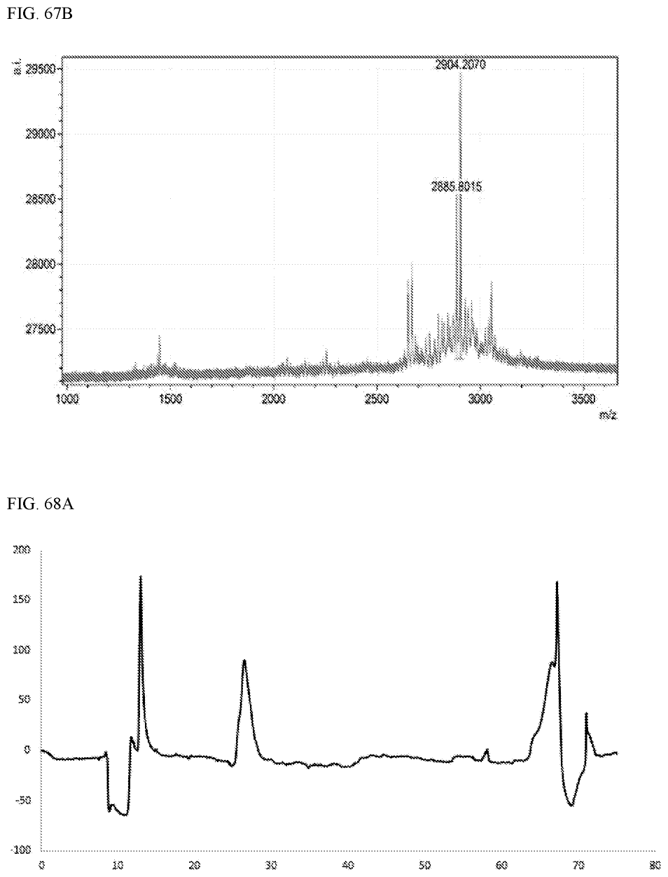

FIG. 67A shows the HPLC analysis result of the peptide complex of Example 66 and FIG. 67B shows the MALDI-TOF mass spectrometry analysis result of the peptide complex of Example 66. It was confirmed that the peptide complex of Example 66 was synthesized successfully with a purity >95%.

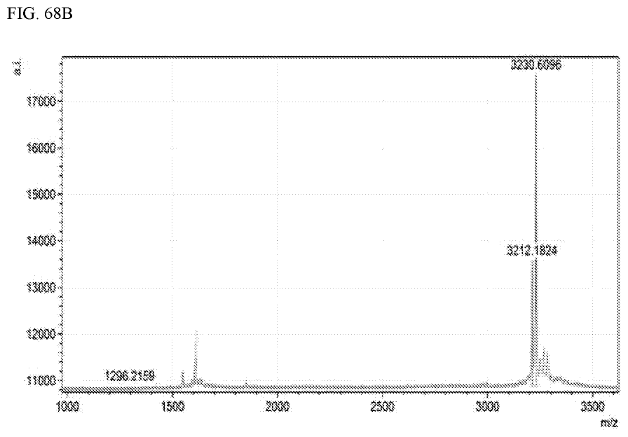

FIG. 68A shows the HPLC analysis result of the peptide complex of Example 67 and FIG. 68B shows the MALDI-TOF mass spectrometry analysis result of the peptide complex of Example 67. It was confirmed that the peptide complex of Example 67 was synthesized successfully with a purity >95%.

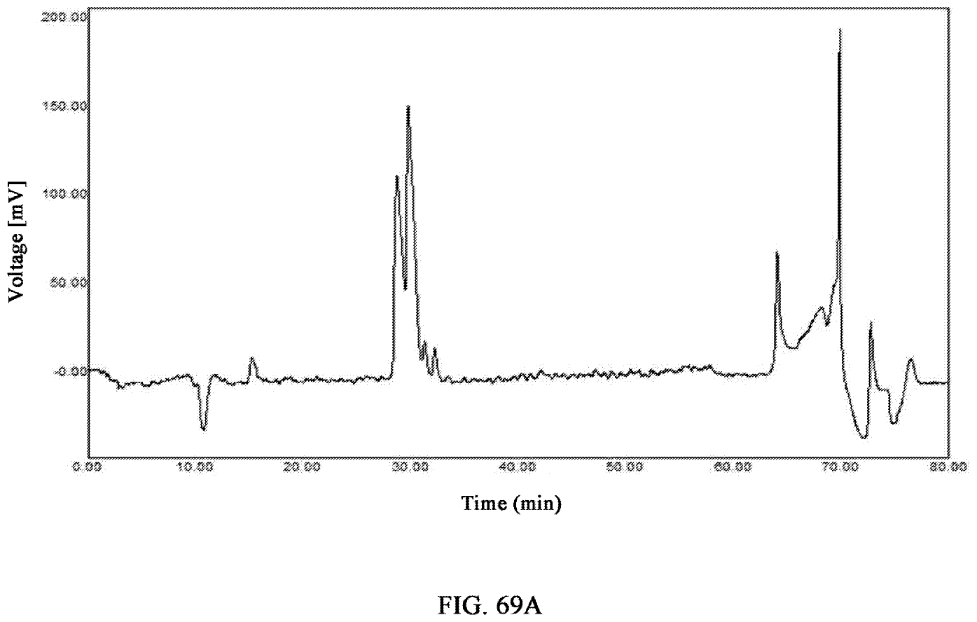

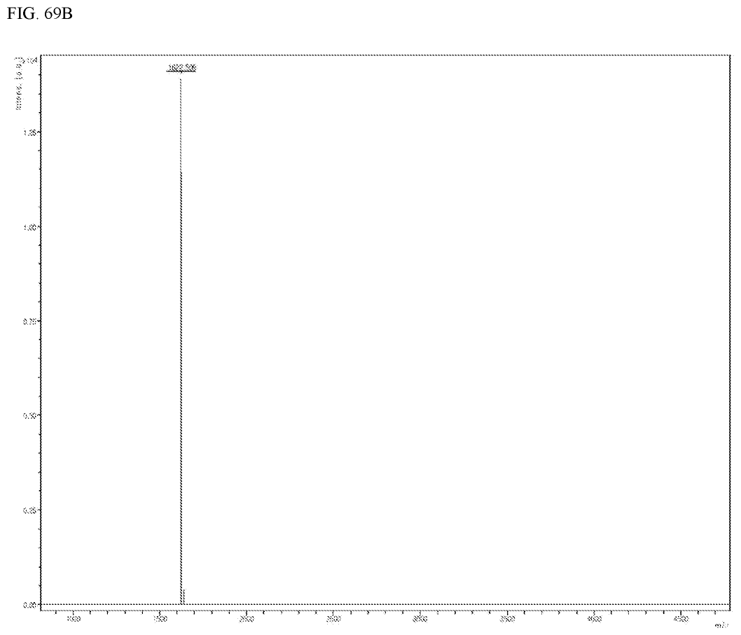

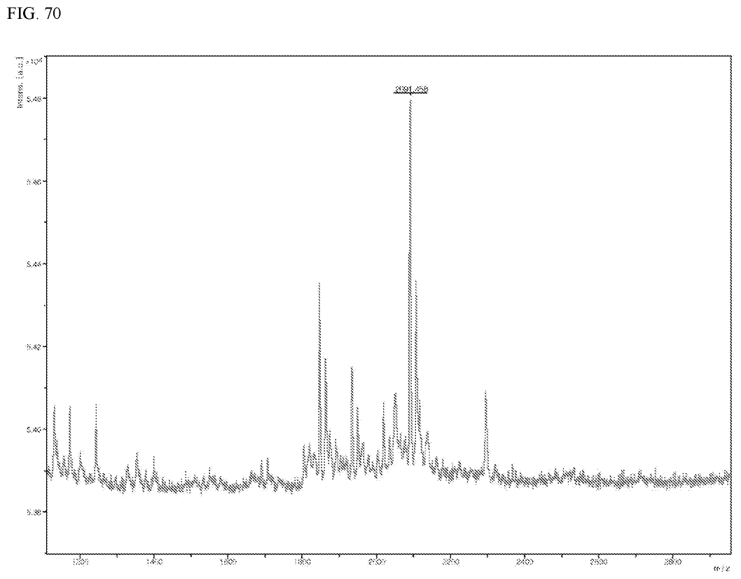

FIG. 69A shows the HPLC analysis result of the peptide complex of Example 52 and FIG. 69B shows the MALDI-TOF mass spectrometry analysis result of the peptide complex of Example 52. It was confirmed that the peptide complex of Example 52 was synthesized successfully with a purity >95%.

FIG. 70 shows the MALDI-TOF mass spectrometry analysis result of the peptide complex of Example 53. It was confirmed that the peptide complex of Example 53 was synthesized successfully.

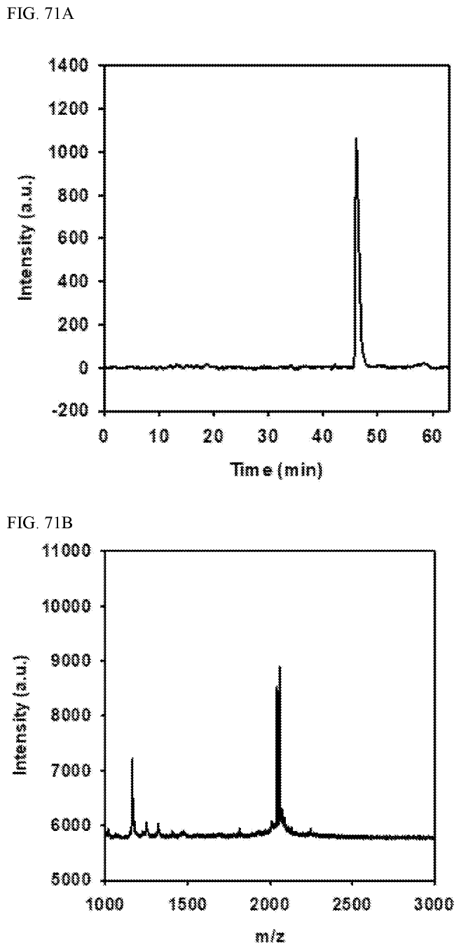

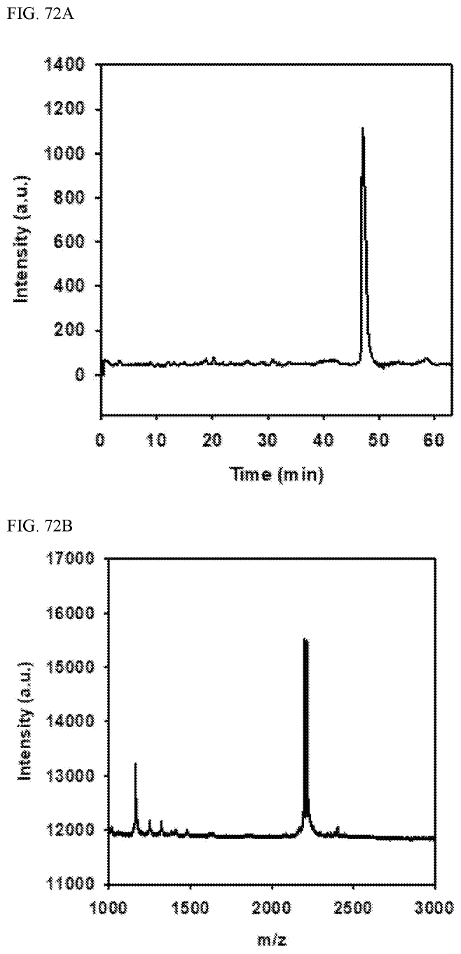

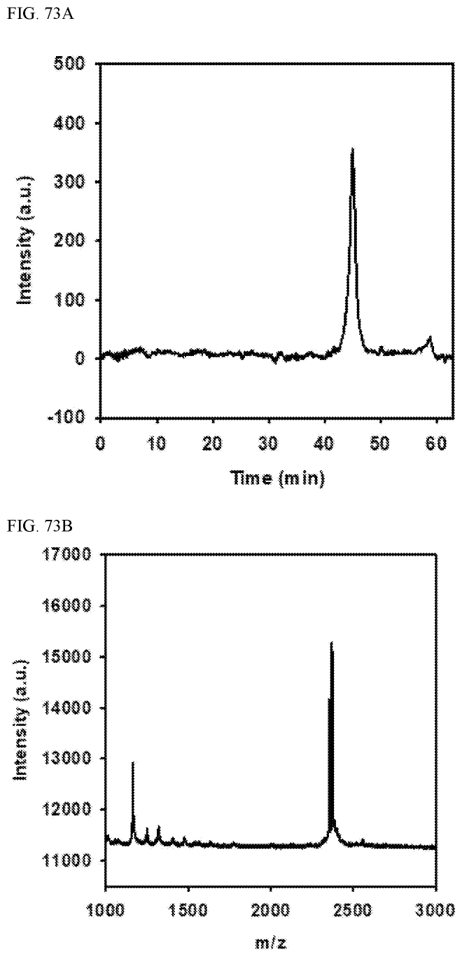

FIG. 71A shows the HPLC analysis result of the peptide complex of Example 46 and FIG. 71B shows the MALDI-TOF mass spectrometry analysis result of the peptide complex of Example 46. It was confirmed that the peptide complex of Example 46 was synthesized successfully. The peptide complex of Example 46 had a molecular weight of 2021.34 g/mol (calculated) and 2041.08 g/mol (observed).

FIG. 72A shows the HPLC analysis result of the peptide complex of Example 47 and FIG. 72B shows the MALDI-TOF mass spectrometry analysis result of the peptide complex of Example 47. It was confirmed that the peptide complex of Example 47 was synthesized successfully with a purity >95%. The peptide complex of Example 47 had a molecular weight of 2177.53 g/mol (calculated) and 2197.30 g/mol (observed).

FIG. 73A shows the HPLC analysis result of the peptide complex of Example 48 and FIG. 73B shows the MALDI-TOF mass spectrometry analysis result of the peptide complex of Example 48. It was confirmed that the peptide complex of Example 48 was synthesized successfully with a purity >95%. The peptide complex of Example 48 had a molecular weight of 2333.71 g/mol (calculated) and 2353.09 g/mol (observed).

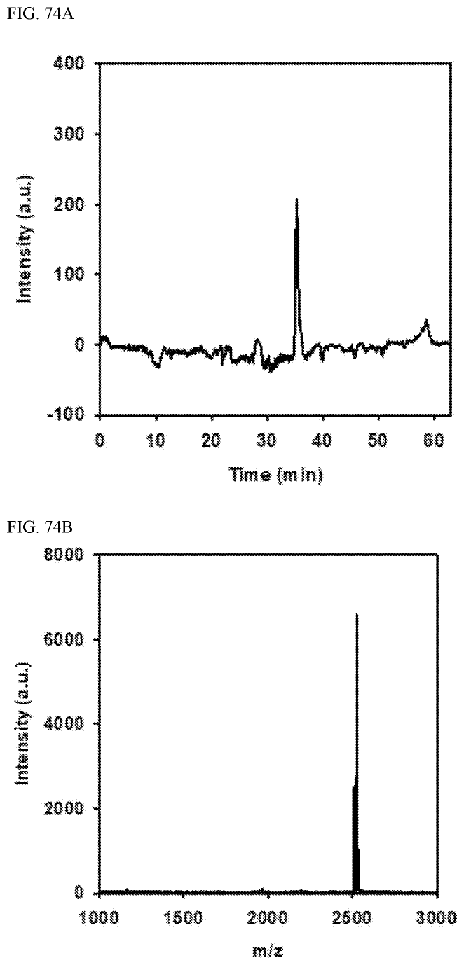

FIG. 74A shows the HPLC analysis result of the peptide complex of Example 49 and FIG. 74B shows the MALDI-TOF mass spectrometry analysis result of the peptide complex of Example 49. It was confirmed that the peptide complex of Example 49 was synthesized successfully with a purity >95%. The peptide complex of Example 49 had a molecular weight of 2489.90 g/mol (calculated) and 2508.38 g/mol (observed).

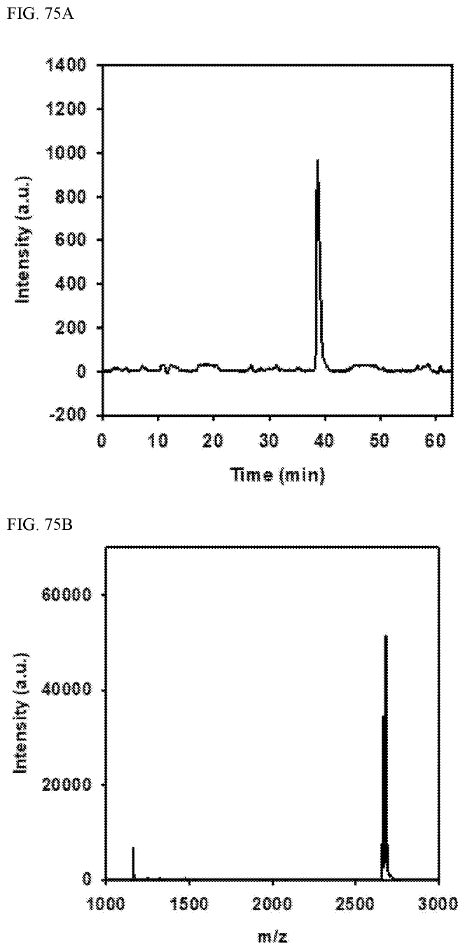

FIG. 75A shows the HPLC analysis result of the peptide complex of Example 50 and FIG. 75B shows the MALDI-TOF mass spectrometry analysis result of the peptide complex of Example 50. It was confirmed that the peptide complex of Example 50 was synthesized successfully with a purity >95%. The peptide complex of Example 50 had a molecular weight of 2646.08 g/mol (calculated) and 2664.37 g/mol (observed).

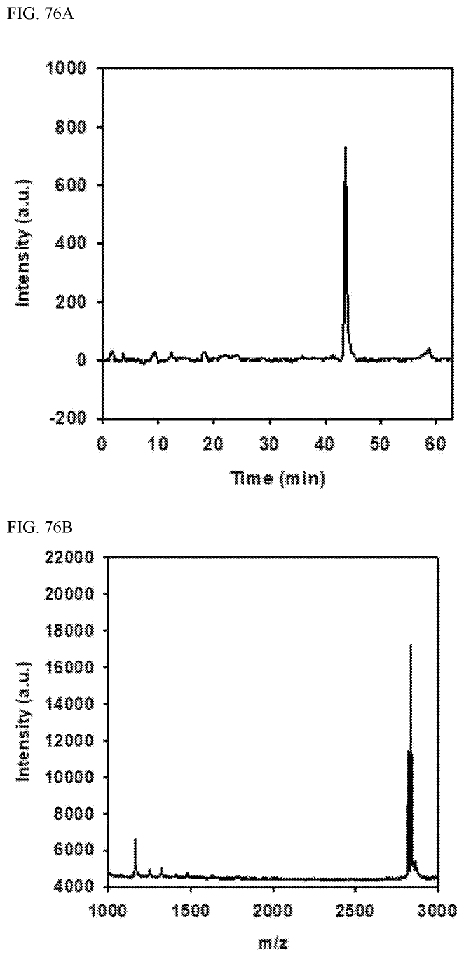

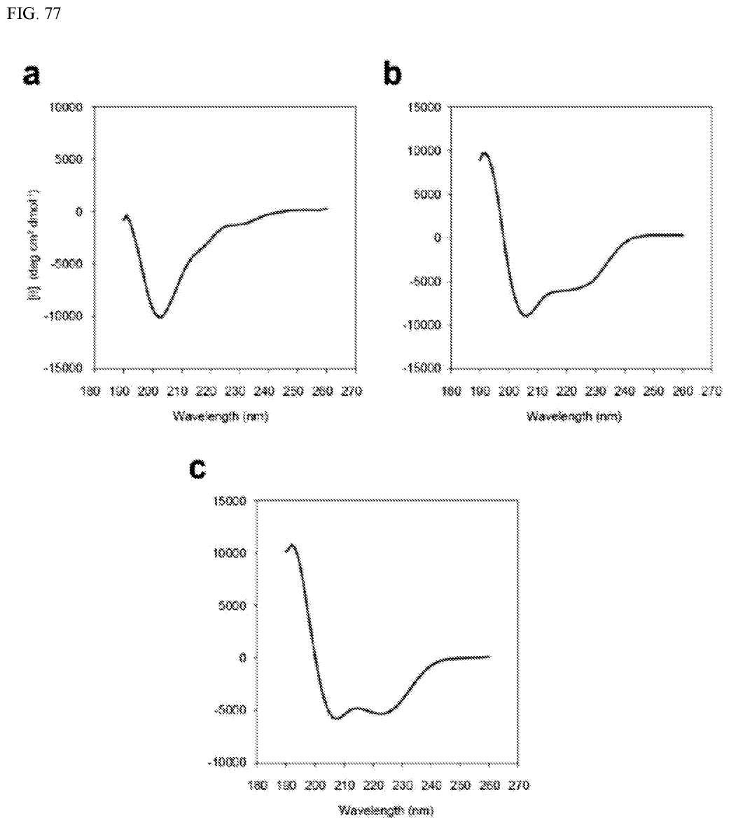

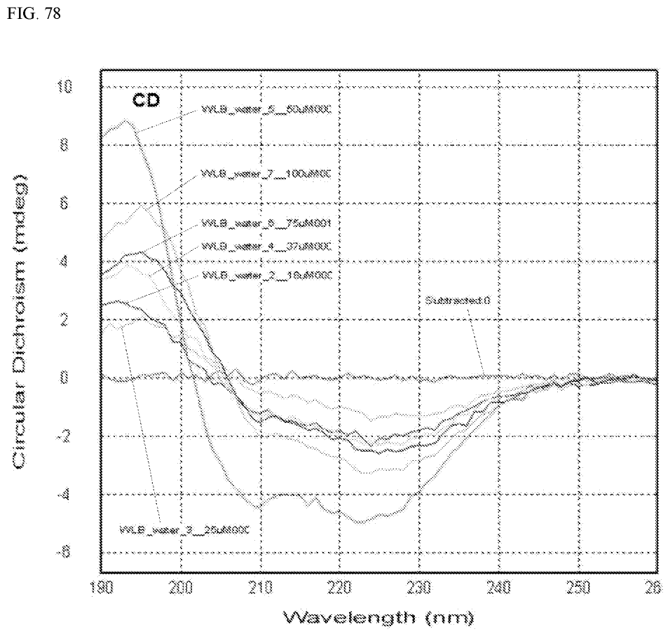

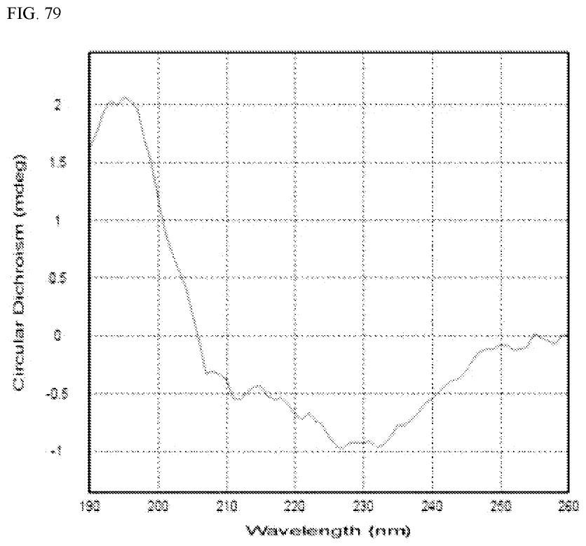

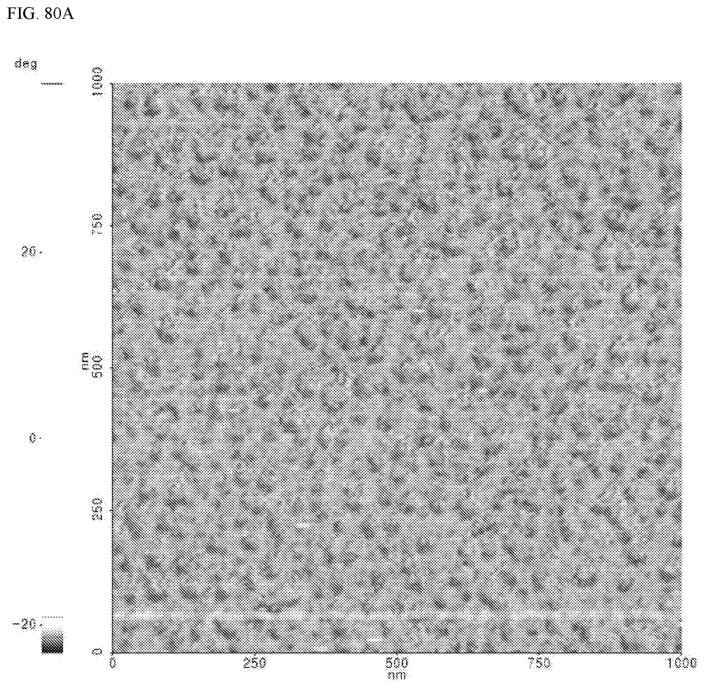

FIG. 76A shows the HPLC analysis result of the peptide complex of Example 51 and FIG. 76B shows the MALDI-TOF mass spectrometry analysis result of the peptide complex of Example 51. It was confirmed that the peptide complex of Example 51 was synthesized successfully with a purity >95%. The peptide complex of Example 51 had a molecular weight of 2802.27 g/mol (calculated) and 2820.75 g/mol (observed).

### Test Example 11. Analysis of α-Helix Structure of Peptide Complexes of Examples 37-97

The CD spectra of the hydrophobic peptides of Examples 37-97 were measured and the θ₂₂₂ₙₘ/θ₂₀₈ₙₘ ratio was analyzed therefrom. The result is shown in Table 11. It can be seen that the helix structure is stabilized when the θ₂₂₂ₙₘ/θ₂₀₈ₙₘ ratio is 1.0-1.2.

TABLE 11

| | θ_{222 nm}/θ_{208 nm} ratio |
|---|---|
| Example 37 | 0.28 |
| Example 38 | 0.44 |
| Example 39 | 0.7 |
| Example 40 | 0.99 |
| Example 41 | 0.91 |
| Example 42 | 1.18 |
| Example 43 | 1.15 |
| Example 44 | 1.1 |
| Example 45 | 1.09 |
| Example 46 | 0.72 |
| Example 47 | 0.84 |
| Example 48 | 0.88 |
| Example 49 | 1.05 |
| Example 50 | 1.10 |
| Example 51 | 1.19 |
| Example 52 | 0.54 |
| Example 53 | 1.11 |
| Example 54 | 1.2 |
| Example 55 | 0.58 |
| Example 56 | 1.11 |
| Example 57 | 1.22 |
| Example 58 | 0.36 |
| Example 59 | 0.77 |
| Example 60 | 1.18 |
| Example 61 | 1.13 |
| Example 62 | 1.08 |
| Example 63 | 1.03 |
| Example 64 | 1.1 |
| Example 65 | 0.94 |
| Example 66 | 0.91 |
| Example 67 | Not determined |
| Example 68 | 0.89 |
| Example 69 | 0.88 |
| Example 70 | 1.14 |
| Example 71 | 1.12 |
| Example 72 | Not determined |
| Example 73 | 1.1 |
| Example 74 | 1.11 |
| Example 75 | Not determined |
| Example 76 | 1.18 |
| Example 77 | 1.09 |
| Example 78 | 1.04 |
| Example 79 | 1.03 |
| Example 80 | 1.2 |
| Example 81 | 1.15 |
| Example 82 | 1.13 |
| Example 83 | 1.08 |
| Example 84 | 1.22 |
| Example 85 | 1.18 |
| Example 86 | 1.14 |
| Example 87 | 1.08 |
| Example 88 | 1.07 |
| Example 89 | 0.99 |
| Example 90 | 1.21 |
| Example 91 | 1.18 |
| Example 92 | 1.2 |
| Example 93 | 1.16 |
| Example 94 | 1.06 |
| Example 95 | 1.2 |
| Example 96 | 1.15 |
| Example 97 | 1.11 |

As shown in Table 11, no minimum was observed at 222 and 208 nm in the RGD spectra of the peptide complexes of Example 67, 72 and 75. From the above results, it was confirmed that most of the peptide complexes according to the present disclosure have a stable α-helix structure. The peptide complexes of Example 37, 47, 52, 55 and 58, wherein the number of the repeat unit a constituting the hydrophobic peptide is 4, had the θ₂₂₂ₙₘ/θ₂₀₈ₙₘ ratio lower than 0.5. Accordingly, it is preferred to use the peptide complexes of Examples 39-45, 48-51, 53-55, 56-57 and 59-97, wherein the number of the repeat unit a constituting the hydrophobic peptide is 6-13.

### Test Example 12. AFM Analysis of Peptide Complexes of Examples 37-40

The peptide complexes of Examples 37-40 were imaged by AFM. 25 μM of the peptide complex was dissolved in distilled water, sonicated for 5 minutes, incubated overnight and dropped on mica for measurement.

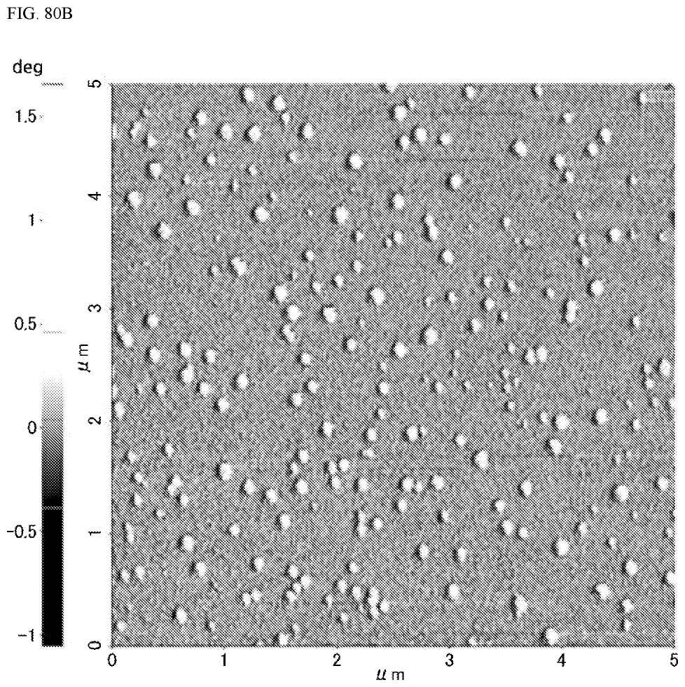

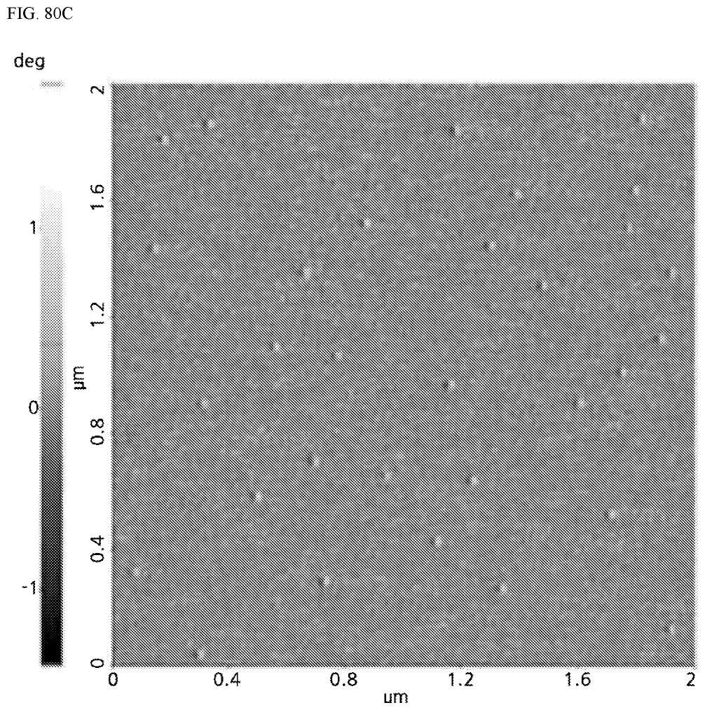

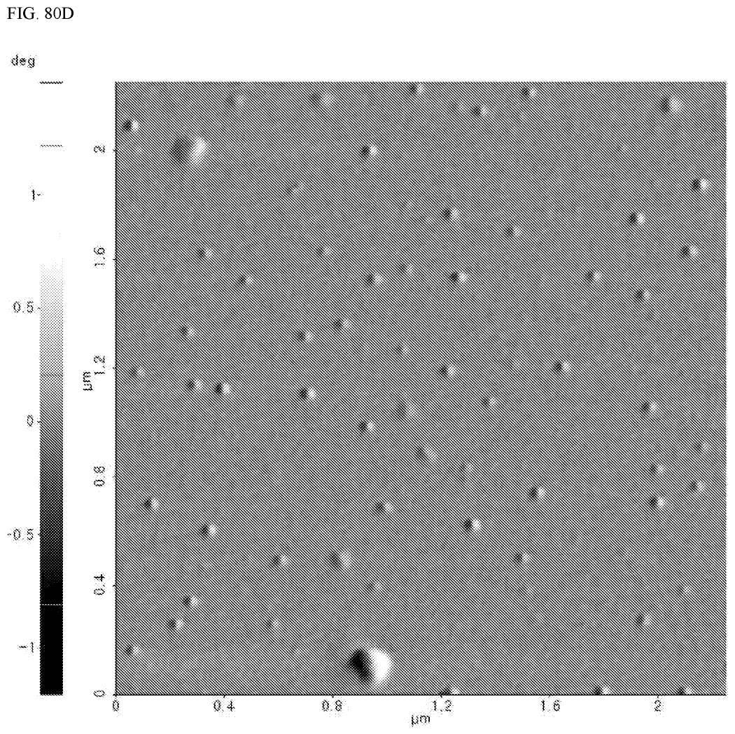

FIGS. 80A-80 show the AFM images of the peptide complexes of Examples 37 (FIG. 80A), 38 (FIG. 80o), 39 (FIG. 800) and 40 (FIG. 80D) and the average diameter of self-assembled structures thereof.

The peptide complexes other than the peptide complexes of Examples 37-40 were also imaged by AFM and their average diameters and the morphologies of self-assembled structures were observed.

TABLE 12

| | AFM morphology | Average diameter (nm) |
|---|---|---|
| Example 37 | Irregular vesicle | — |
| Example 38 | Vesicle | 50-200 |
| Example 39 | Micelle | 20 |
| Example 40 | Micelle | 20-30 |
| Example 41 | Micelle | 20-40 |
| Example 52 | Not assembled | — |
| Example 53 | Micelle | 20-30 |
| Example 54 | Micelle, vesicle | 20-90 |
| Example 55 | Not assembled | — |
| Example 56 | Micelle | 20-40 |
| Example 57 | Vesicle | 30-80 |
| Example 58 | Not assembled | — |
| Example 59 | Vesicle | 160-410 |
| Example 60 | Vesicle | 100-130 |
| Example 61 | Vesicle | 90-250 |
| Example 62 | Vesicle | 110-150 |
| Example 63 | Micelle | 20-50 |
| Example 64 | Micelle | 30-40 |
| Example 65 | Micelle | 20 |
| Example 66 | Micelle | 20-30 |
| Example 68 | Vesicle | 40-110 |
| Example 69 | Vesicle | 40-260 |
| Example 70 | Vesicle | 50 |
| Example 71 | Vesicle | 50-100 |
| Example 76 | Vesicle | 50-160 |
| Example 77 | Vesicle | 50-190 |
| Example 78 | Vesicle | 80-320 |
| Example 79 | Vesicle | 450-500 |
| Example 80 | Micelle | 20-30 |
| Example 81 | Micelle | 20-30 |
| Example 82 | Vesicle | 120-140 |
| Example 83 | Vesicle | 40-80 |
| Example 88 | Vesicle | 100-140 |
| Example 89 | Vesicle | 70-80 |
| Example 90 | Micelle | 20-30 |
| Example 91 | Micelle | 20-30 |
| Example 92 | Vesicle | 200 |
| Example 93 | Vesicle | 180-360 |
| Example 95 | Vesicle | 240-280 |
| Example 96 | Vesicle | 130-190 |

As seen from FIGS. 80A-800, it was confirmed that the peptide complex of Example 37 exists as irregular aggregates and vesicles and the peptide complex of Example 38 exists as spherical particles. In particular, it was confirmed that the peptide complex of Example 39 exists as micelle particles with a length of 6-10 nm, an average diameter of 20 nm and an average height of 2-4 nm. It was confirmed that the peptide complex of Example 40 exists as micelle particles with a length of 7.2-12.4 nm and an average diameter of 20 nm.

As shown in Table 12, it can be seen that the hydrophobic peptide having an α-helix structure according to the present disclosure successfully self-assembles into spherical particles in liquid not only with the RGD hydrophilic peptide but also with the hydrophilic polymer such as PEG, etc.

In addition, it can be seen that the peptide complex according to the present disclosure self-assembles into spherical nanoparticles with an average diameter of 20-400 nm in an aqueous solvent.

However, because irregular aggregation occurs when the number of the repeat unit of the hydrophobic peptide having α-helices is 6 or smaller, it is preferred to use the hydrophobic peptide having α-helices wherein the number of the repeat unit is 6 or larger.

### Test Example 13. Peptide-DNA Nanostructure of Peptide Complex of Example 60 and dsDNA

1) Linear dsDNA

A large-scale plasmid DNA (pTWIN-Rev; 7,528 bp) was prepared using the NucleoBond Xtra Maxi Plus kit (Macherey-Nagel, Germany). The plasmid DNA was digested into linear double-stranded DNA with the BamH1 restriction enzyme (New England Biolabs, USA). The linear plasmid DNA was purified from the restriction digest using 1.1% agarose gel. Electrophoresis was conducted at 120 V for 180 minutes in a 1× TBE buffer. After the electrophoresis, the DNA was stained with the SYBR Safe DNA gel stain (Invitrogen, USA). A slice of the DNA band that corresponds to 7.5-kb linear dsDNA was cut out and the linear plasmid DNA was extracted using the MEGA Quick-Spin™ Plus kit (iNtRON, Korea). The purity of the DNA was found to be high (A₂₆₀/A₂₈₀ ratio was 1.8 and A₂₆₀/A₂₃₀ ratio was 2.0-2.2) as measured with a Nanodrop 1000 spectrophotometer (Thermo Scientific, USA).

### 2) Peptide-DNA Nanostructure

The peptide complex of Example 60 was dissolved in distilled water and then sonicated. An R-α₁₀-PEG₁₆/dsDNA nanostructure was prepared by slowly adding dsDNA (1.8 ng/μL) of the same volume to the peptide complex. After reaction at room temperature for 2-3 minutes, the nanostructure was analyzed first by AFM and TEM. The second AFM and TEM analyses were performed after 12 days of reaction. The third AFM and TEM analyses were performed after 22 days of reaction.

### 3) DNase I Protection Assay

60 ng of dsDNA (2 μL; 30 ng/μL) or R-α₁₀-PEG₁₆/dsDNA nanostructure (artificial chromosome form) was mixed with 0.1 μL (0.2 unit) of DNase I and 1 μL of a 10× DNase I reaction buffer. The volume of the mixture was 10 μL. After 20 minutes of incubation at 37° C., 0.3 μL of 0.05 M EDTA was added to inactivate the enzyme. Then, the sample was mixed with 1.7 μL of 60% glycerol and electrophoresed on 1.1% agarose gel (120 minutes, 100 V). For visualization, the DNA was stained with an SYBR Safe DNA gel stain (Invitrogen, USA). To evaluate the time-dependent kinetics of DNA degradation, UV absorbance at 260 nm was recorded over time after the addition of DNase I. Each sample (71.2 μL) containing DNA (30 ng/μL) and a 10× DNase I reaction buffer (8 μL) was mixed in a cuvette. Then, DNase I (0.8 μL) was added to the cuvette and the sample was mixed by pipetting for about 90-105 seconds. UV absorbance at 260 nm (A₂₆₀) was measured using a V-650 UV-vis spectrophotometer (JASCO, Japan). A quartz cuvette with a path length of 10 mm was used. The absorbance was recorded at intervals of 10 seconds.

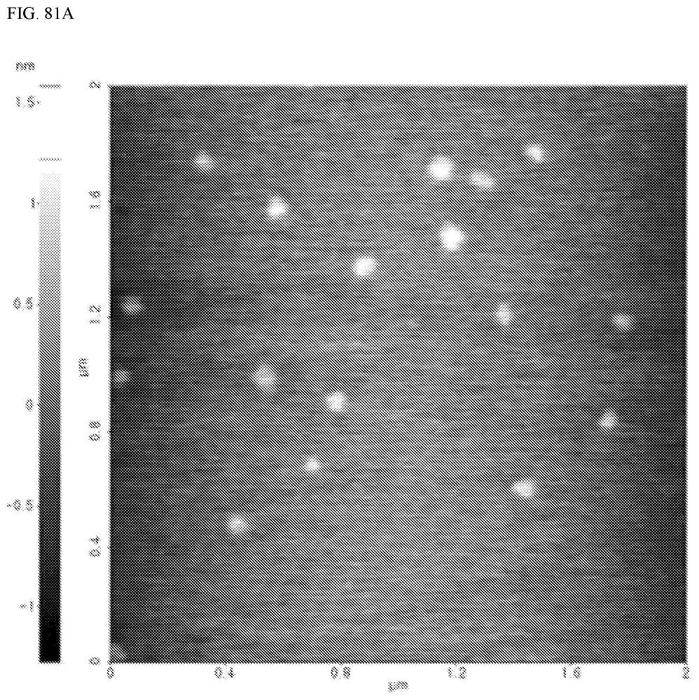

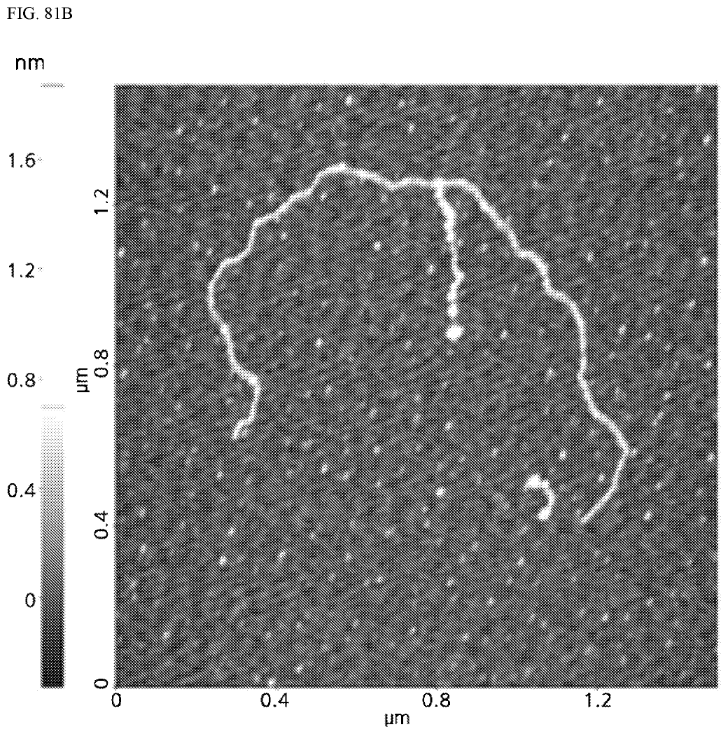

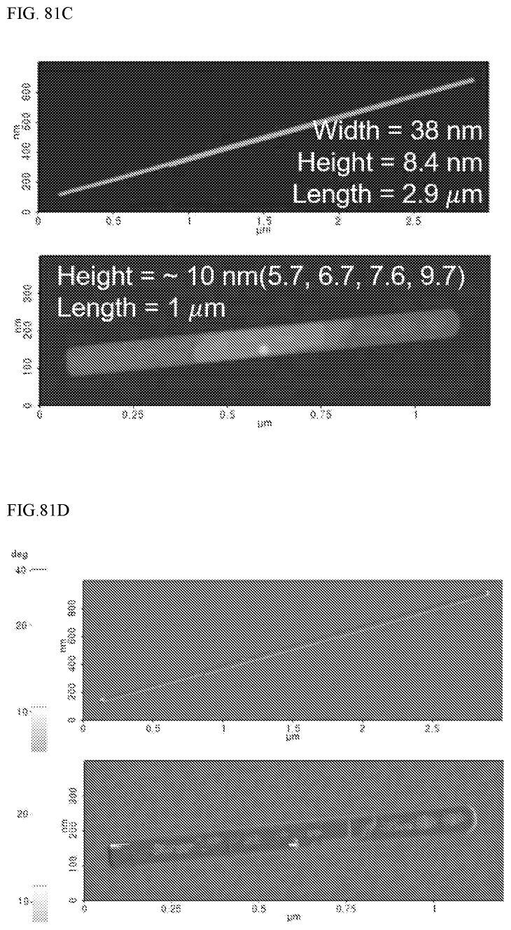

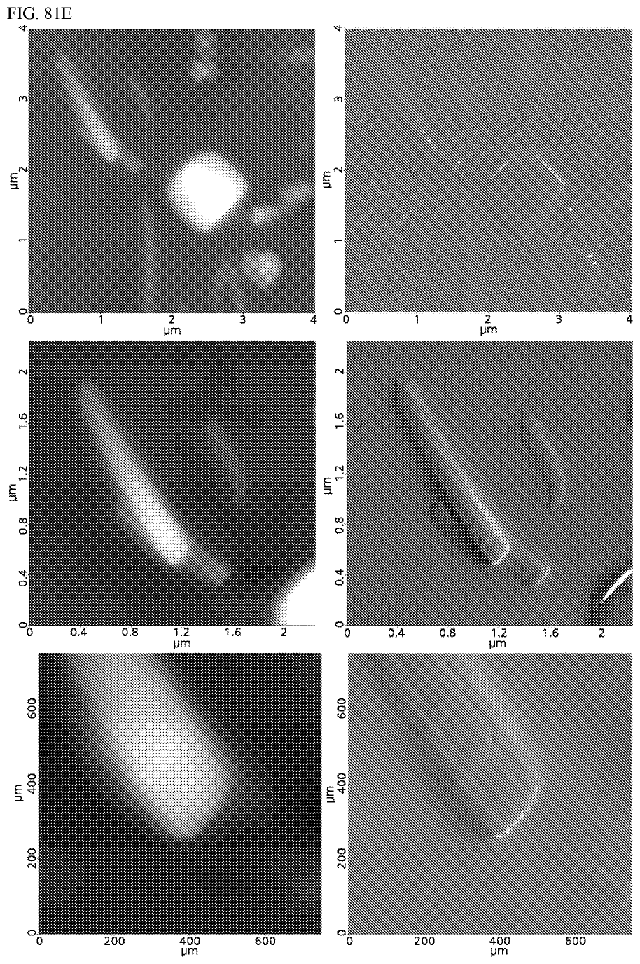

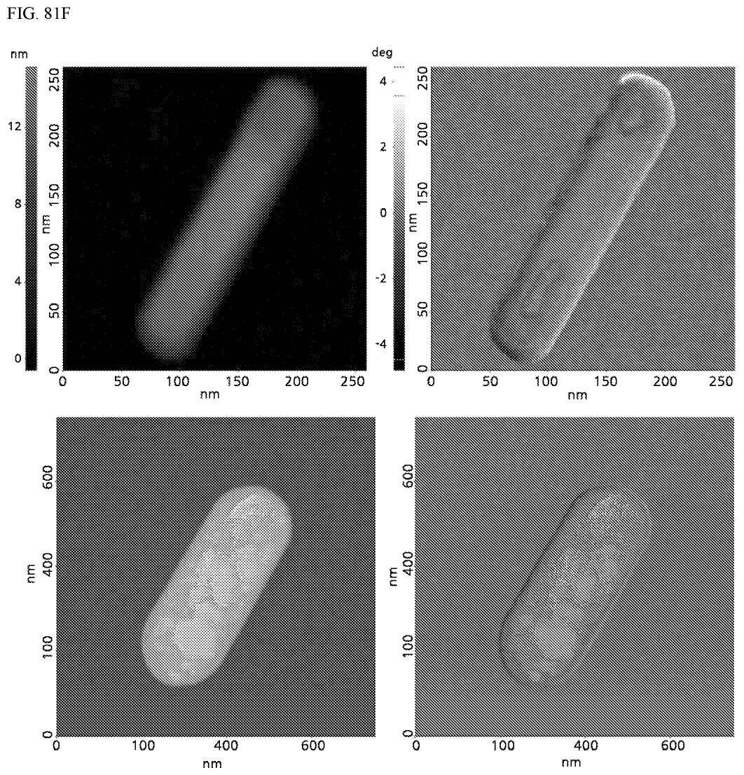

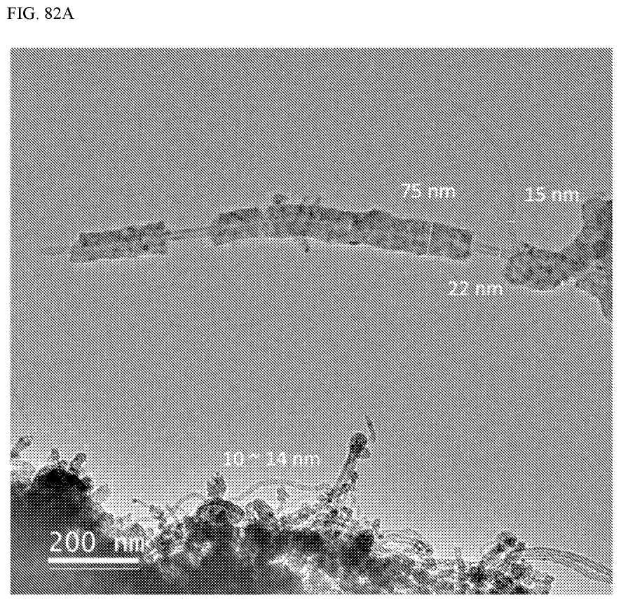

FIG. 81A shows the AFM image of the peptide complex of Example 60 (R-α₁₀-PEG₁₆) and FIG. 81B shows the AFM image of dsDNA. FIGS. 81C and 81D show the AFM images of the R-α₁₀-PEG₁₆/dsDNA nanostructure obtained 2-3 minutes after the preparation, FIG. 81E shows the AFM image of the R-α₁₀-PEG₁₆/dsDNA nanostructure obtained 12 days after the preparation and FIG. 81F shows the AFM image of the R-α₁₀-PEG₁₆/dsDNA nanostructure obtained 22 days after the preparation.

As shown in FIGS. 81A and 81B, it was confirmed that the peptide complex of Example 60 (R-α₁₀-PEG₁₆) has a length of 1-1.25 nm and the dsDNA is a long linear structure with a width of 23 nm and a length of 2.2-2.3 μm.

As shown in FIGS. 81C and 81D, it was confirmed that the R-α₁₀-PEG₁₆/dsDNA nanostructure self assembles into a nanoribbon form with a width of 33-48 nm, a height of 6-10 nm and a length of 0.8-3 μm.

As shown in FIG. 81E, the R-α₁₀-PEG₁₆/dsDNA nanostructure was changed into a short and thick nanoribbon structure with a width of 234-1172 nm and a height of 26-132 nm 12 days later. It was confirmed that the R-α₁₀-PEG₁₆/dsDNA nanostructure finally forms an ‘artificial chromosome’-like structure as the dsDNA is surrounded by the peptide complex (R-α₁₀-PEG₁₆).

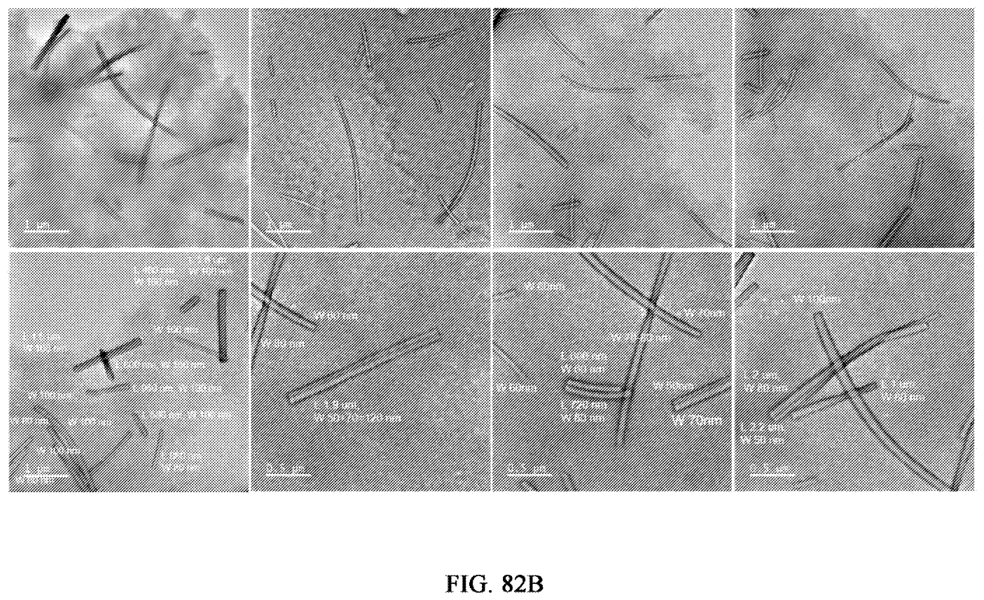

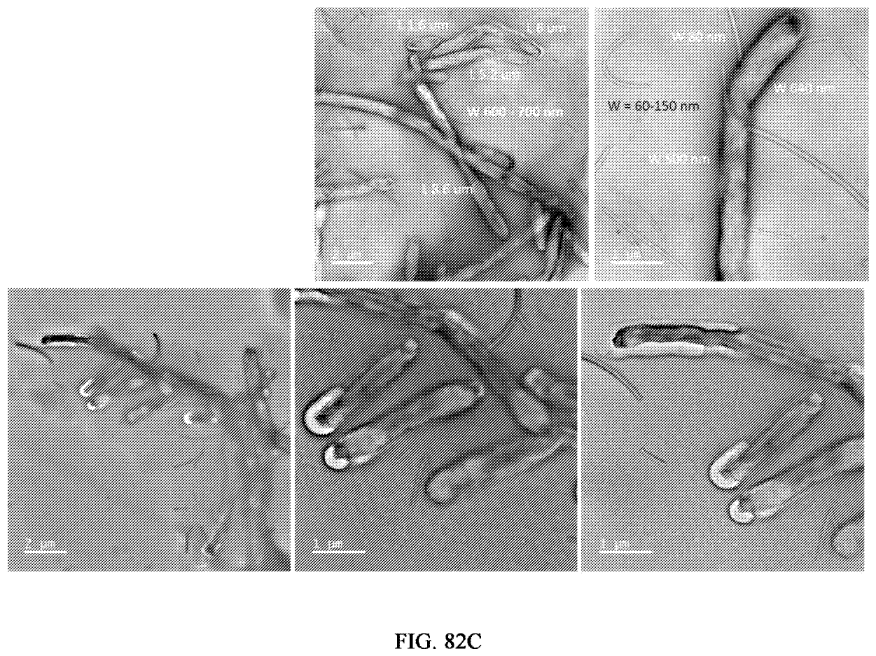

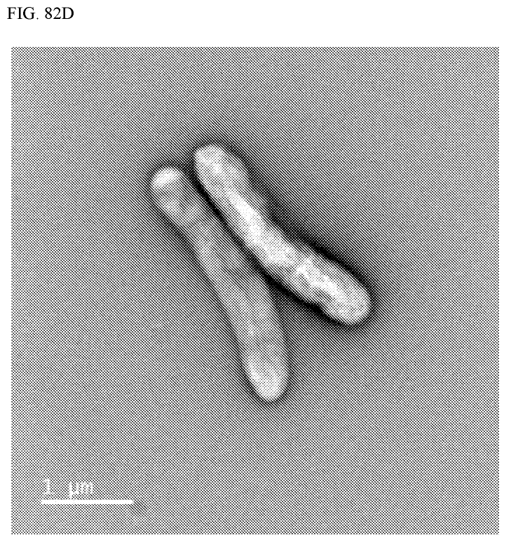

FIG. 82A shows the TEM image of the R-α₁₀-PEG₁₆/dsDNA nanostructure obtained 2-3 minutes after preparation, FIG. 82B shows the TEM image of the R-α₁₀-PEG₁₆/dsDNA nanostructure obtained 12 days after the preparation and FIGS. 82C and 82D show the TEM image of the R-α₁₀-PEG₁₆/dsDNA nanostructure obtained 22 days after the preparation.

As shown in FIGS. 82A-82D, it was confirmed that the R-α₁₀-PEG₁₆/dsDNA nanostructure, which was a long linear structure initially, self-assembled into a long nanoribbon form over time and, finally, formed an ‘artificial chromosome’-like nanostructure (width: 600 nm, length: 2-3 μm) as the width was increased and the length was decreased gradually.

Based on these results, the α-helix structure of the peptide complex of Example 60 (R-α₁₀-PEG₁₆), the dsDNA and the R-α₁₀-PEG₁₆/dsDNA nanostructure was compared.

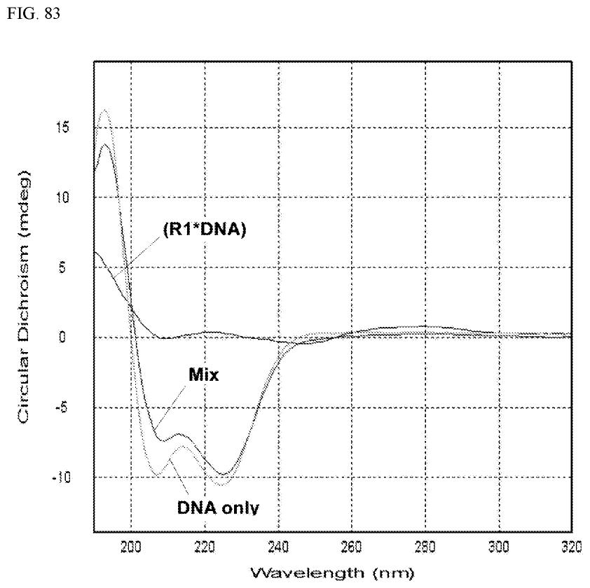

FIG. 83 shows the CD spectra of the peptide complex of Example 60 (R-α₁₀-PEG₁₆), the dsDNA and the R-α₁₀-PEG₁₆/dsDNA nanostructure. It was confirmed that the peptide complex of Example 60 (R-α₁₀-PEG₁₆) and the R-α₁₀-PEG₁₆/dsDNA nanostructure maintain a stable α-helix structure. That is to say, it can be seen that the R-α₁₀-PEG₁₆/dsDNA nanostructure includes the α-helix structure within the complex. In the figure, R1 only indicates the peptide complex of Example 60 (R-α₁₀-PEG₁₆) alone and Mix indicates the R-α₁₀-PEG₁₆/dsDNA nanostructure.

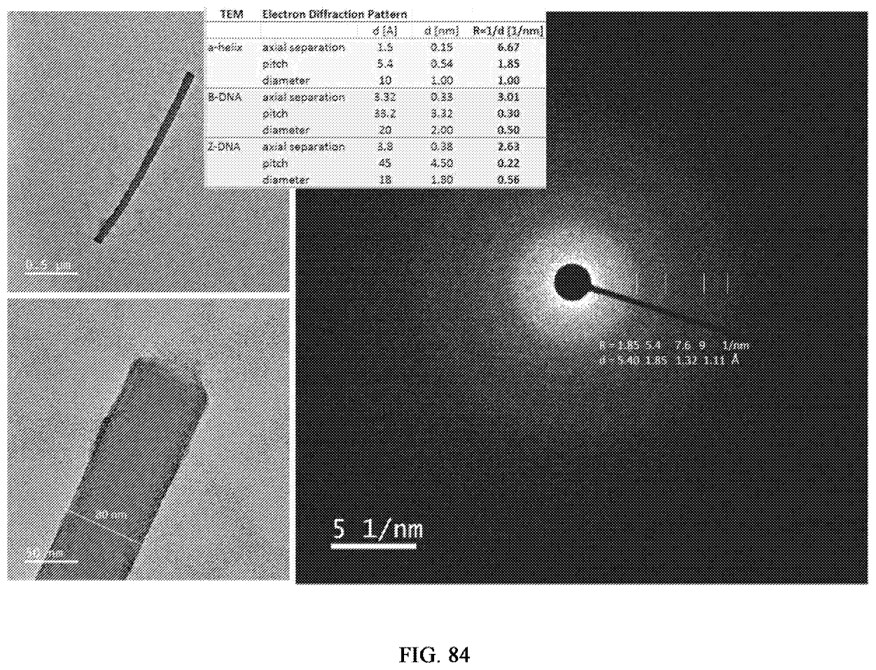

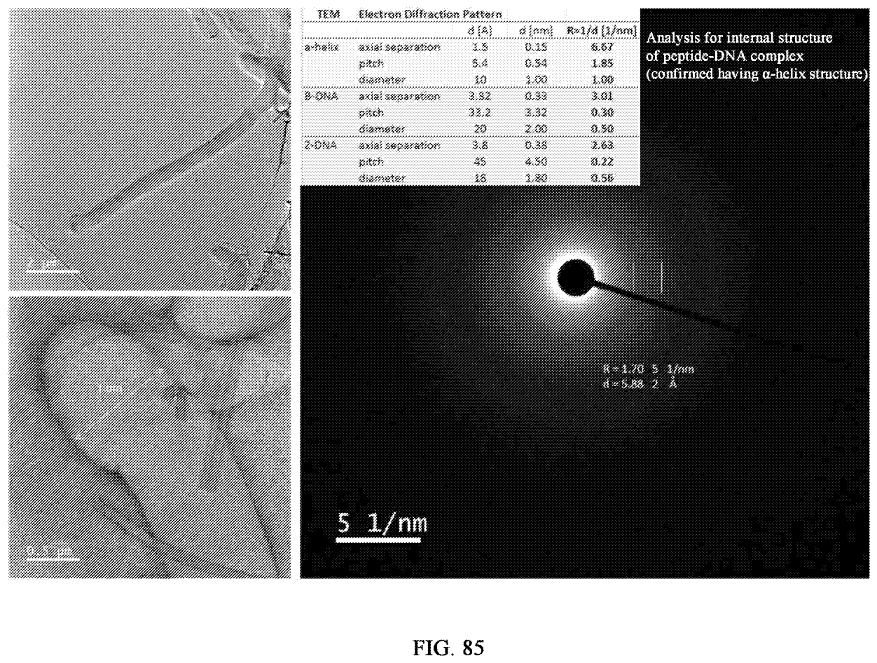

FIG. 84 shows the SAED (selected area electron diffraction) pattern of the R-α₁₀-PEG₁₆/dsDNA nanostructure having a long nanoribbon structure and FIG. 85 shows the SAED (selected area electron diffraction) pattern of the R-α₁₀-PEG₁₆/dsDNA nanostructure having an artificial chromosome structure. It can be seen that the R-α₁₀-PEG₁₆/dsDNA nanostructure includes the α-helix structure within the R-α₁₀-PEG₁₆/dsDNA nanostructure.

It was investigated whether the R-α₁₀-PEG₁₆/dsDNA nanostructure also protects DNA stably from enzymatic degradation through the DNase I protection assay described above.

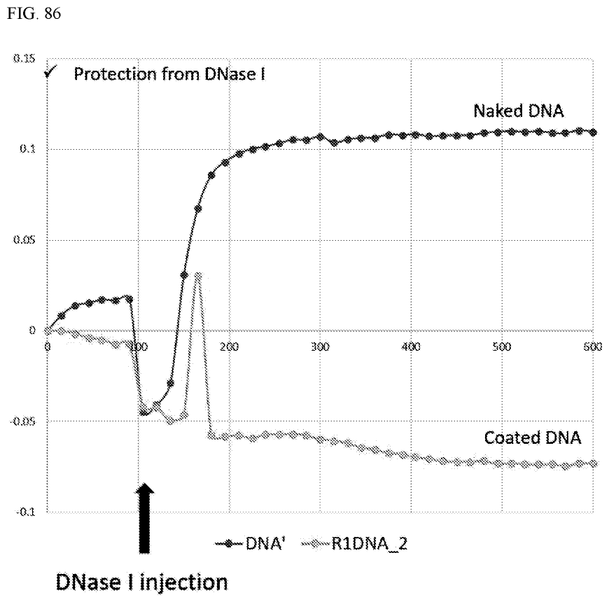

FIG. 86 shows the UV-vis measurement result obtained after adding DNase I to dsDNA and the R-α₁₀-PEG₁₆/dsDNA nanostructure. It can be seen that whereas the dsDNA was degraded completely in naked state (naked DNA), the dsDNA was maintained without degradation when it exists in the R-α₁₀-PEG₁₆/dsDNA nanostructure.

### Test Example 14. Analysis of Peptide Complex of Example 63 (R₄-α₁₀-PEG₁₆)

1) Linear dsDNA

First, a large-scale plasmid DNA (pTWIN-Rev; 7,528 bp) was prepared using the NucleoBond Xtra Maxi Plus kit (Macherey-Nagel, Germany). The plasmid DNA was digested into linear double-stranded DNA with the BamH1 restriction enzyme (New England Biolabs, USA). The linear plasmid DNA was purified from the restriction digest using 1.1% agarose gel. Electrophoresis was conducted at 120 V for 180 minutes in a 1× TBE buffer. After the electrophoresis, the DNA was stained with the SYBR Safe DNA gel stain (Invitrogen, USA). A slice of the DNA band that corresponds to 7.5-kb linear dsDNA was cut out and the linear plasmid DNA was extracted using the MEGA Quick-Spin™ Plus kit (iNtRON, Korea). The purity of the DNA was found to be high (A₂₆₀/A₂₈₀ ratio was 1.8 and A₂₆₀/A₂₃₀ ratio was 2.0-2.2) as measured with a Nanodrop 1000 spectrophotometer (Thermo Scientific, USA).

### 2) Peptide-DNA Nanostructure

The peptide complex of Example 63 was dissolved in distilled water and then sonicated. An R₄-α₁₀-PEG₁₆/dsDNA nanostructure was prepared by slowly adding dsDNA (145.2 ng/μL in H₂O) of the same volume to the peptide complex at a concentration of 22.6 μM. After reaction at room temperature for 40 minutes, the nanostructure was analyzed first by AFM and TEM. The second AFM and TEM analyses were performed after 111 days of reaction.

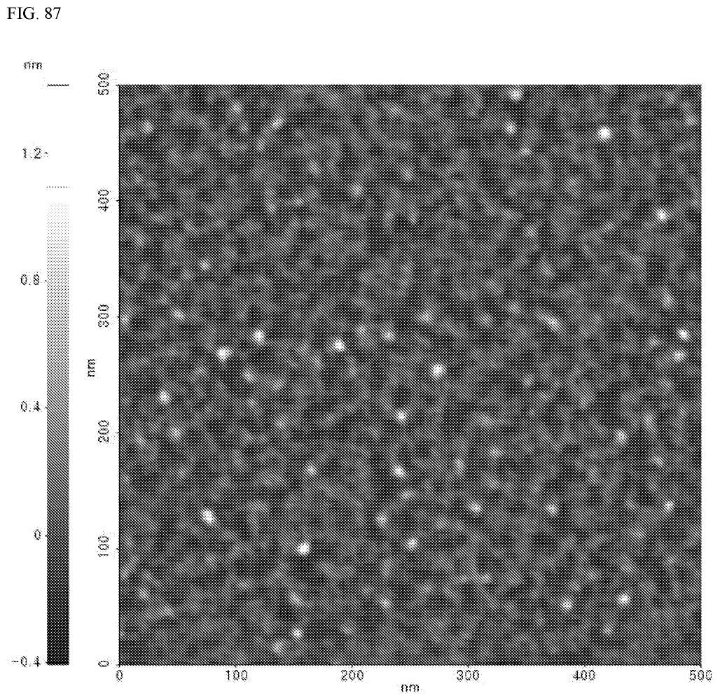

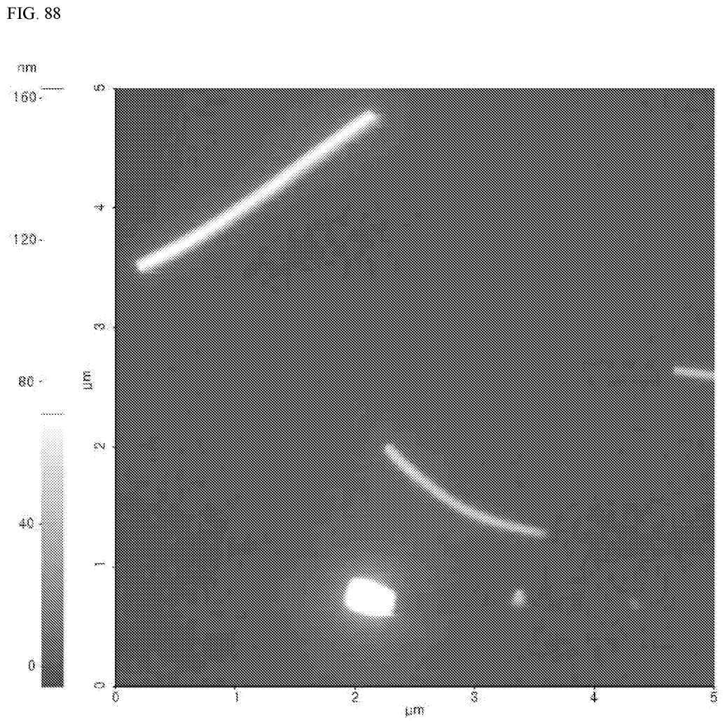

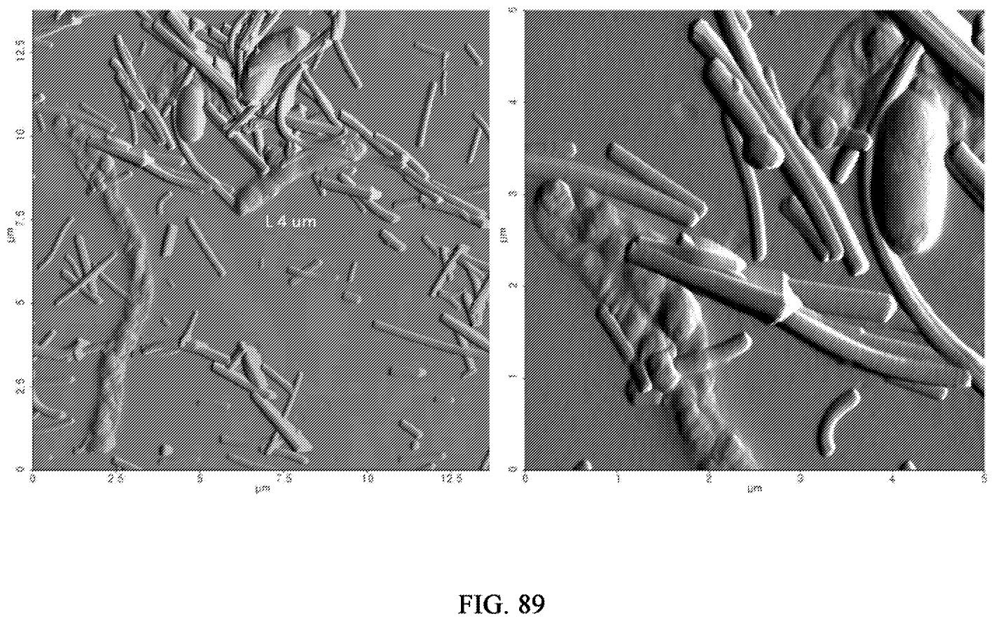

FIG. 87 shows the AFM image of the peptide complex of Example 63, FIG. 88 shows the AFM image of the R₄-α₁₀-PEG₁₆/dsDNA nanostructure obtained 40 minutes after preparation and FIG. 89 shows the AFM image of the R₄-α₁₀-PEG₁₆/dsDNA nanostructure obtained 111 days after the preparation.

From FIG. 87, it can be seen that the peptide complex of Example 63 self-assembles into spherical particles in an aqueous solvent.

From FIG. 88 and FIG. 89, it can be seen that the peptide complex of Example 63 forms a nanostructure in nanoribbon form through self-assembly when mixed with dsDNA and then forms a nanostructure in artificial chromosome form. It can be seen that, although it contains more positively charged amino acids than the peptide complex of Example 60, it is prepared into the nanostructure according to the present disclosure.

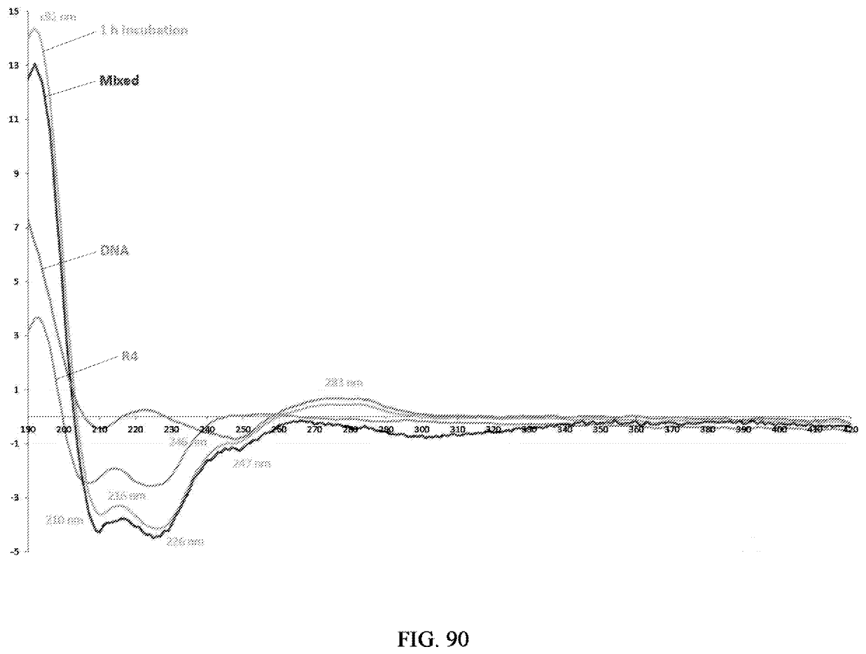

FIG. 90 shows the CD spectrum of the R₄-α₁₀-PEG₁₆/dsDNA nanostructure. It can be seen that the positively charged peptide complex of Example 63 also forms a nanostructure via cooperative interaction with dsDNA. In the figure, R4 indicates the peptide complex of Example 63, Mix indicates the R₄-α₁₀-PEG₁₆/dsDNA nanostructure, and 1 h incubation indicates the R₄-α₁₀-PEG₁₆/dsDNA nanostructure incubated for 1 hour. It was confirmed that the R₄-α₁₀-PEG₁₆/dsDNA nanostructure retains the α-helix structure of the peptide and the DNA structure even after the formation into the artificial chromosome form.

### Test Example 15. Analysis of Nanostructures Prepared from Peptide Complex of Example 60 and Various Genetic Materials

1) Preparation of Plasmid DNA, Linear dsDNA and ssDNA

First, a large-scale plasmid DNA (pTWIN-Rev; 7,528 bp) was prepared using the NucleoBond Xtra Maxi Plus kit (Macherey-Nagel, Germany). The plasmid DNA was digested into linear double-stranded DNA with the BamH1 restriction enzyme (New England Biolabs, USA) and purified using 1.1% agarose gel. Electrophoresis was conducted at 120 V for 180 minutes in a 1× TBE buffer. After the electrophoresis, the DNA was stained with the SYBR Safe DNA gel stain (Invitrogen, USA). A slice of the DNA band that corresponds to 7.5-kb linear dsDNA was cut out and the linear plasmid DNA was extracted using the MEGA Quick-Spin™ Plus kit (iNtRON, Korea). The purity of the DNA was found to be high (A₂₆₀/A₂₃₀ ratio was 1.8 and A₂₆₀/A₂₃₀ ratio was 2.0-2.2) as measured with a Nanodrop 1000 spectrophotometer (Thermo Scientific, USA).

As ssDNA, a short oligomer with a length of 20 nt prepared by Bioneer was used (SEQ ID NO 120).

### 2) Peptide-DNA Nanostructures

The peptide complex of Example 63 was dissolved in distilled water and then sonicated. R-α₁₀-PEG₁₆/dsDNA and R-α₁₀-PEG₁₆/ssDNA nanostructures with the charge ratio (+/−) of the nucleic acid material and the peptide being 0, 0.03, 0.06, 0.12, 0.25, 0.5, 1, 2, 4, 8, 16, 32, 64 and 128 were prepared by slowly injecting dsDNA (30 ng/μL in H₂O) or ssDNA (30 ng/μL in H₂O) at various concentrations to the peptide complex of Example 60. EMSA was performed after 1 hour of incubation.

### 3) EMSA

For EMSA (electrophoretic mobility shift assay), 10% glycerol was added to the sample. Electrophoresis was conducted at 100 V for 120 minutes on 1% agarose gel. The band was stained with the SYBR Safe DNA gel stain (Invitrogen, USA) for visualization.

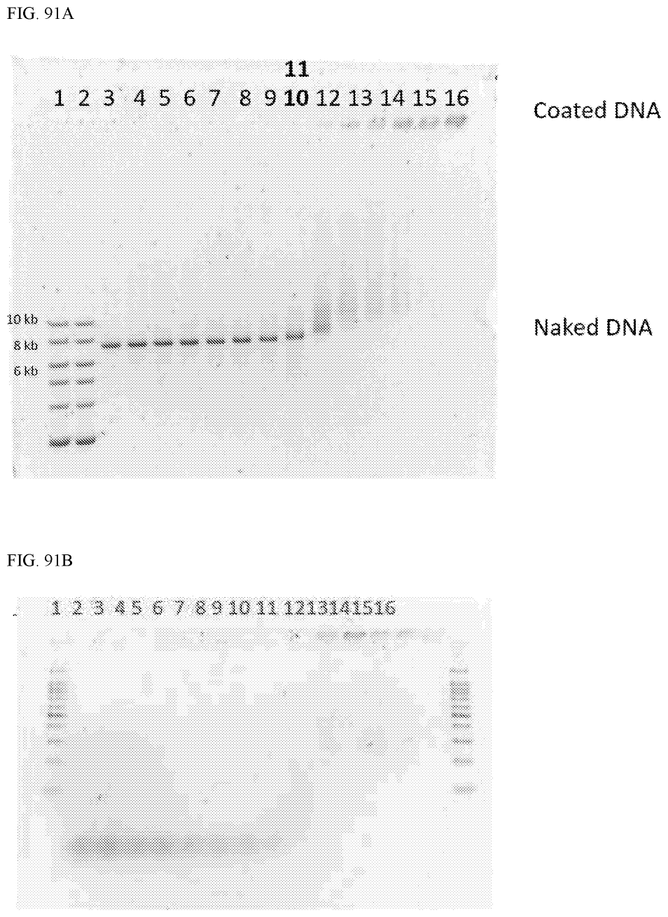

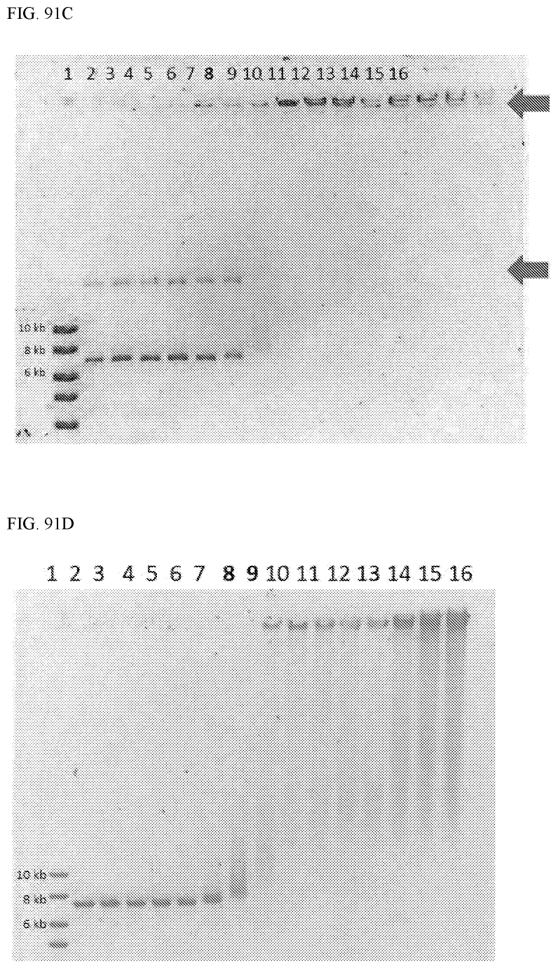

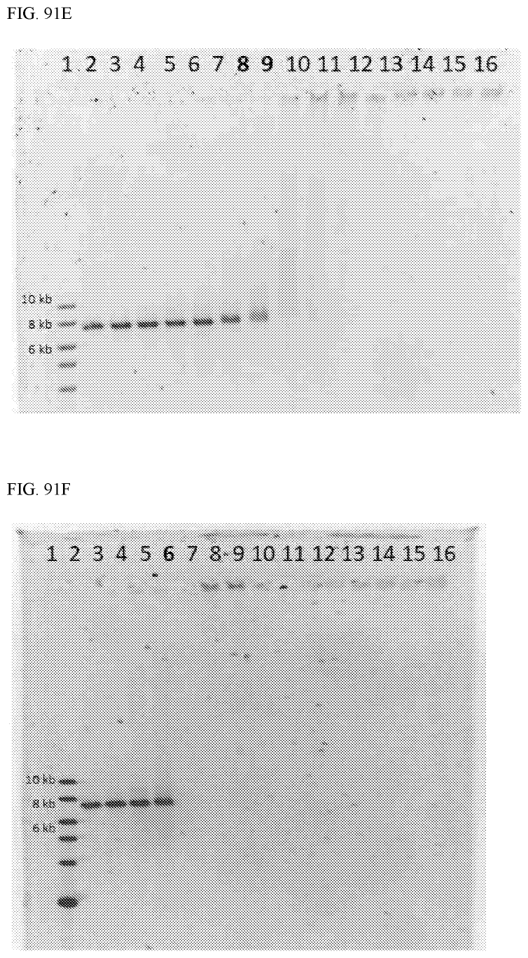

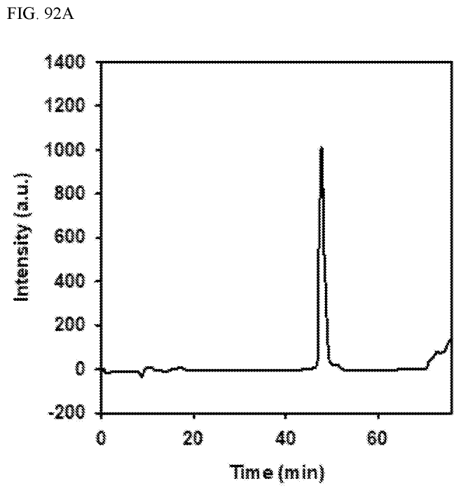

FIG. 91A shows the EMSA result for the nanostructure of the peptide complex of Example 60 (R-α₁₀-PEG₁₆) and dsDNA and FIG. 91B shows the EMSA result for the nanostructure of the peptide complex of Example 60 (R-α₁₀-PEG₁₆) and ssDNA. In the figures, the ladders indicate the nanostructures prepared by mixing the nucleic acid material (dsDNA or ssDNA) and the peptide complex at a charge ratio (+/−) of 0, 0.03, 0.06, 0.12, 0.25, 0.5, 1, 2, 4, 8, 16, 32, 64 and 128.

From FIGS. 91A and 91B, it was confirmed that the peptide complex according to the present disclosure forms a nanostructure by interacting with a nucleic acid material regardless of the kind and length of the nucleic acid material.

### Test Example 16. Analysis of Nanostructures of Peptide Complexes of Examples 61-63 and Linear Plasmid DNA

Peptide-DNA nanostructures were prepared by mixing the peptide complexes of Examples 61-63 with dsDNA. A peptide-DNA nanostructure was prepared by mixing the peptide complex of Example 61 with a plasmid DNA (circular dsDNA, pTWIN-rev=7.5 kbp). The preparation process was the same as in Test Example 15. The R-α₁₀-PEG₁₆/pDNA, R-α₁₀-PEG₁₆/dsDNA nanostructures were prepared by mixing the nucleic acid material and the peptide at a charge ratio (+/−) of 0, 0.03, 0.06, 0.12, 0.25, 0.5, 1, 2, 4, 8, 16, 32, 64, 128 and 256. EMSA was performed after incubation for 1.5 hours.

For EMSA (electrophoretic mobility shift assay), 10% glycerol was added to the sample. The electrophoresis was performed for 120 minutes at 100 Von 1% agarose gel. The band was visualized by staining with the SYBR Safe DNA gel stain (Invitrogen, USA).

FIG. 91C shows the EMSA result for the nanostructure of the peptide complex of Example 61 (R-α₁₀-PEG₁₆) and pDNA, FIG. 91D shows the EMSA result for the nanostructure of the peptide complex of Example 61 (R-α₁₀-PEG₁₆) and dsDNA, FIG. 91E shows the EMSA result for the nanostructure of the peptide complex of Example 62 (R-α₁₀-PEG₁₆) and dsDNA and FIG. 91F shows the EMSA result for the nanostructure of the peptide complex of Example 63 (R-α₁₀-PEG₁₆) and dsDNA. In the figures, the ladders indicate the nanostructures prepared by mixing the nucleic acid materials (dsDNA, ssDNA) and the peptide complex at different charge ratios (+/−) of 0, 0.03, 0.06, 0.12, 0.25, 0.5, 1, 2, 4, 8, 16, 32, 64, 128 and 256.

As shown in FIGS. 91C to 91F, it was confirmed that the peptide polymer of Examples 61-63 according to the present disclosure also successfully form nanostructures by binding to nucleic acid materials.

### Test Example 17. Structural Analysis of Cyclic Peptide Complexes

The cyclic peptide complexes of Examples 98-111 were purified by HPLC and analyzed by MALDI-TOF and CD spectroscopy. The HPLC condition was as follows: C4 semiprep TFA ACN 15-35% 10-60 min, mobile phase: ACN (TFA 0.1%), gradient: 15-35%/10-60 min (+0.4%/min), flow rate: 2.000 mL/min, injection volume: 4.5 mL, injection solvent: ACN 5%, temperature: 25° C. UV detection: 230 nm.

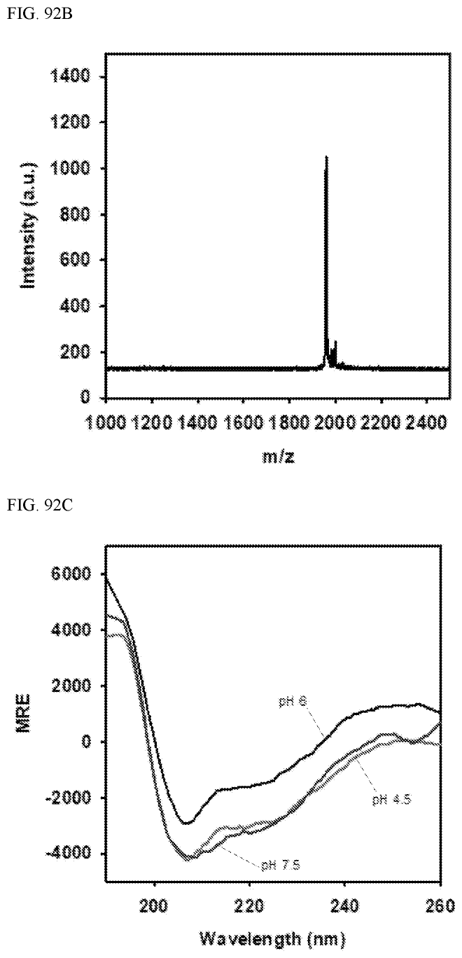

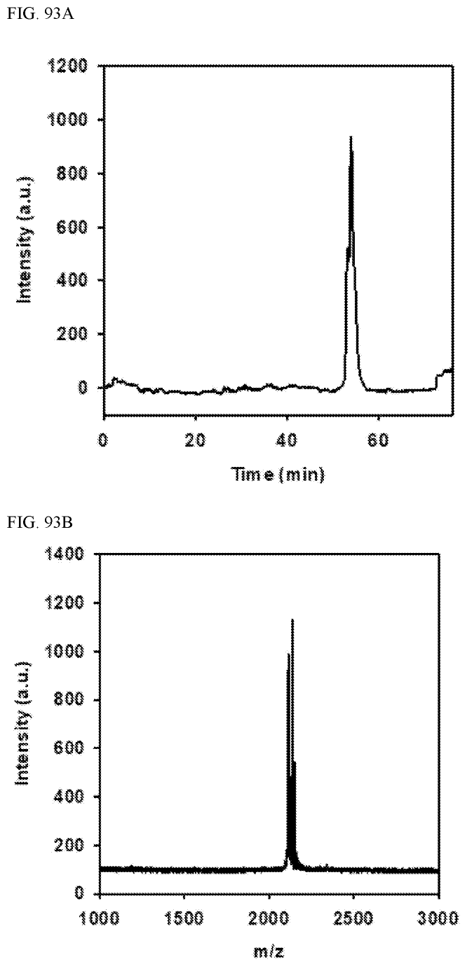

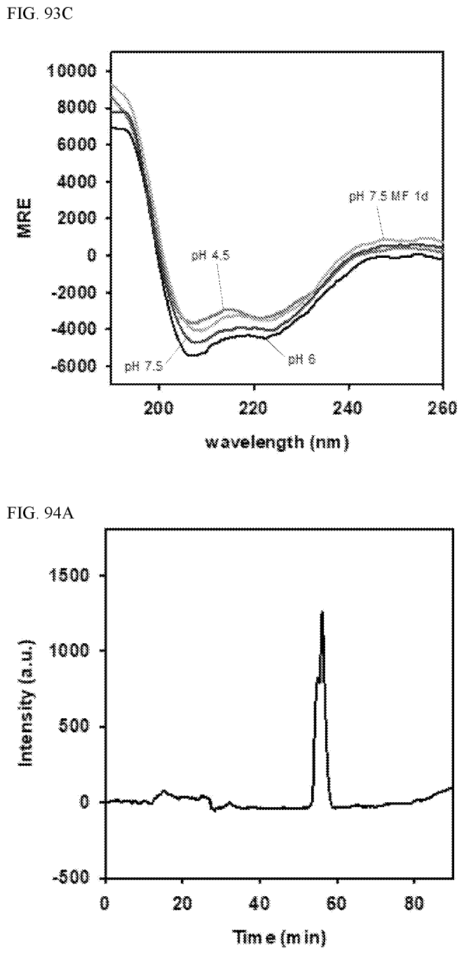

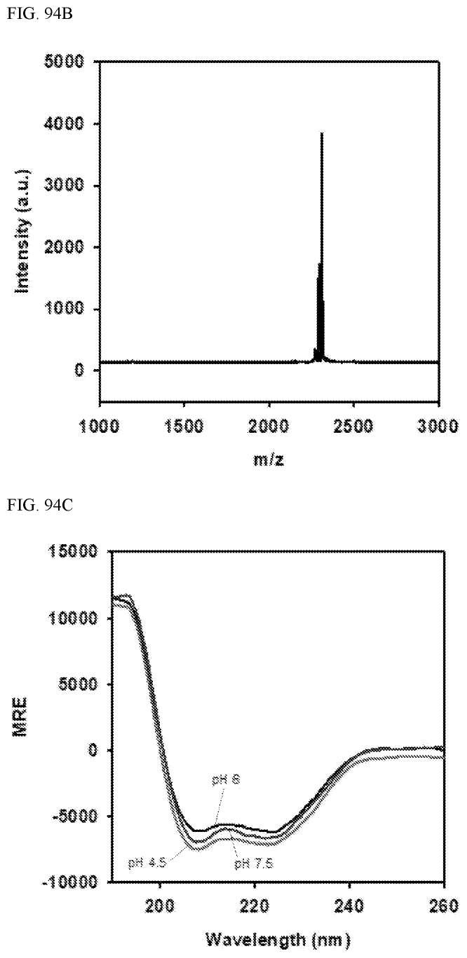

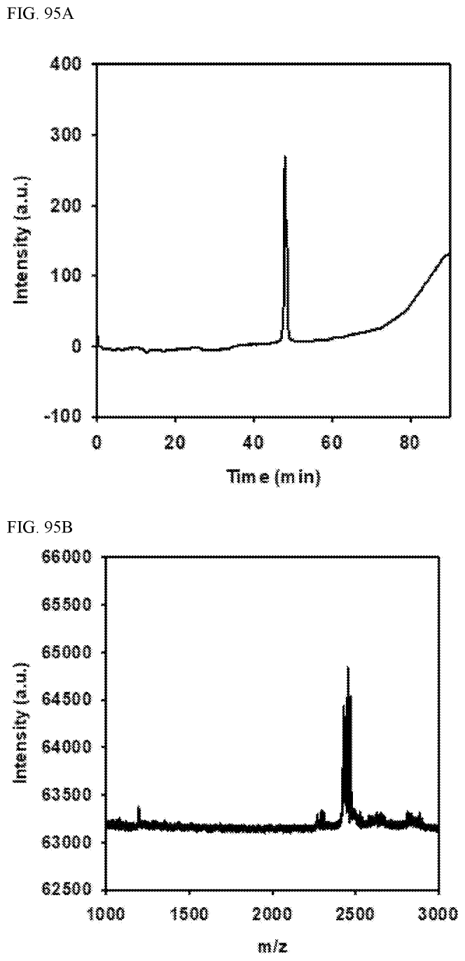

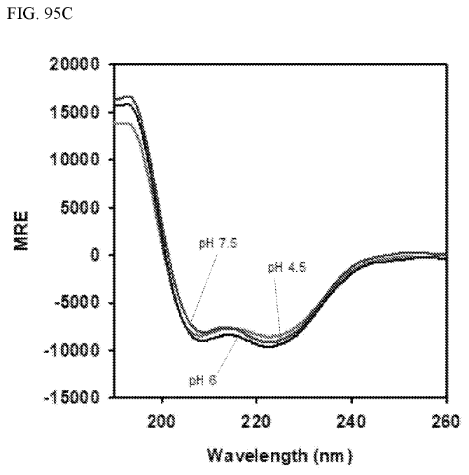

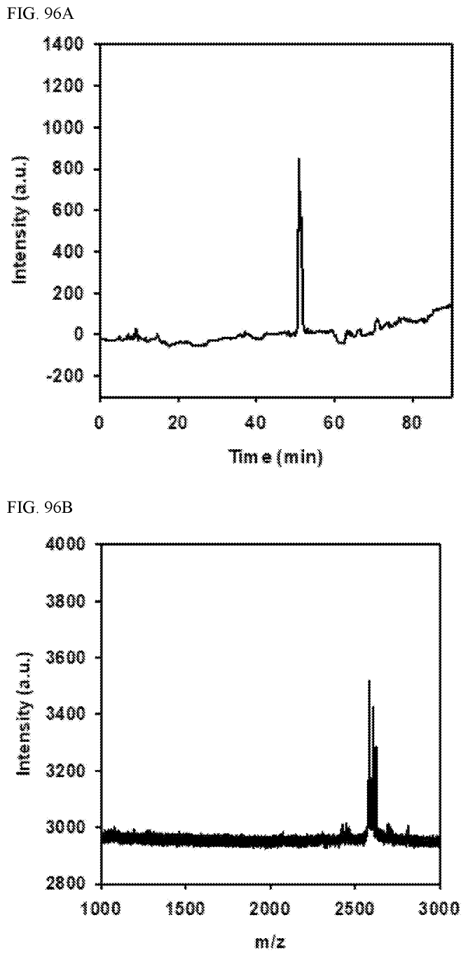

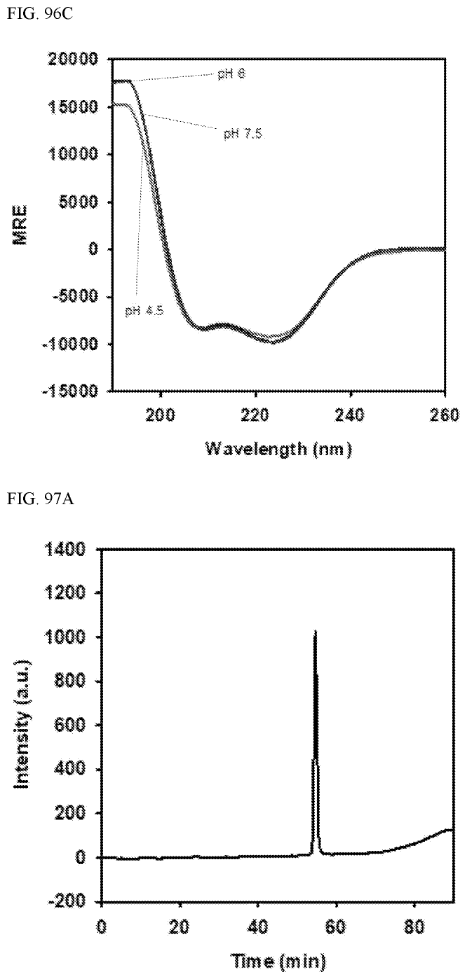

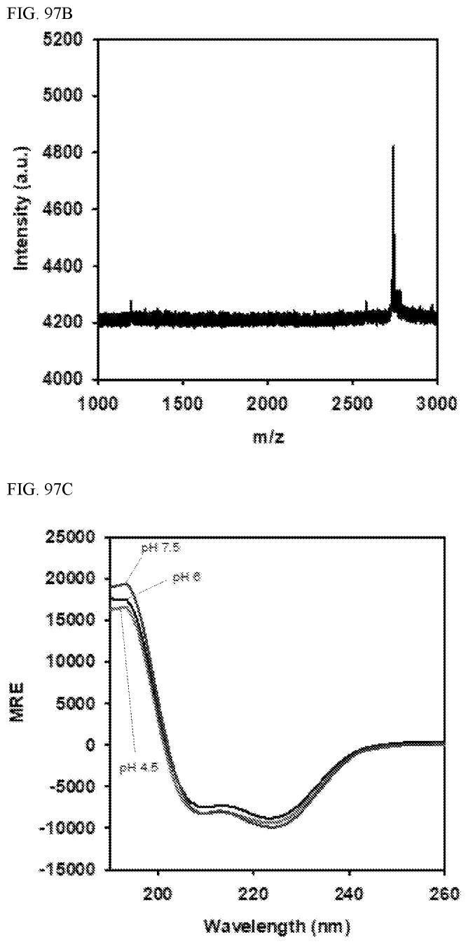

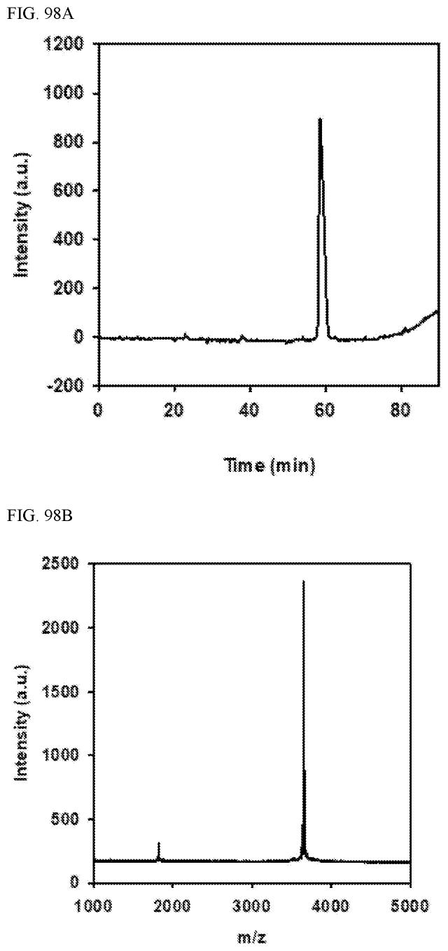

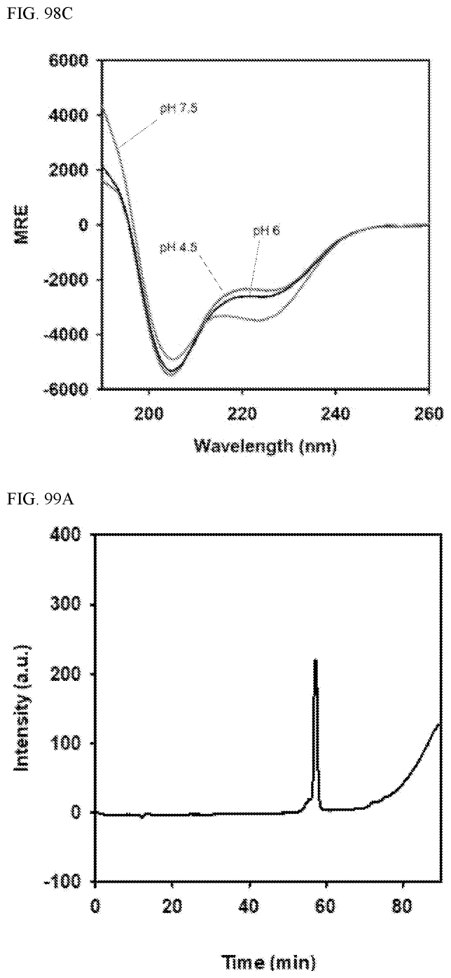

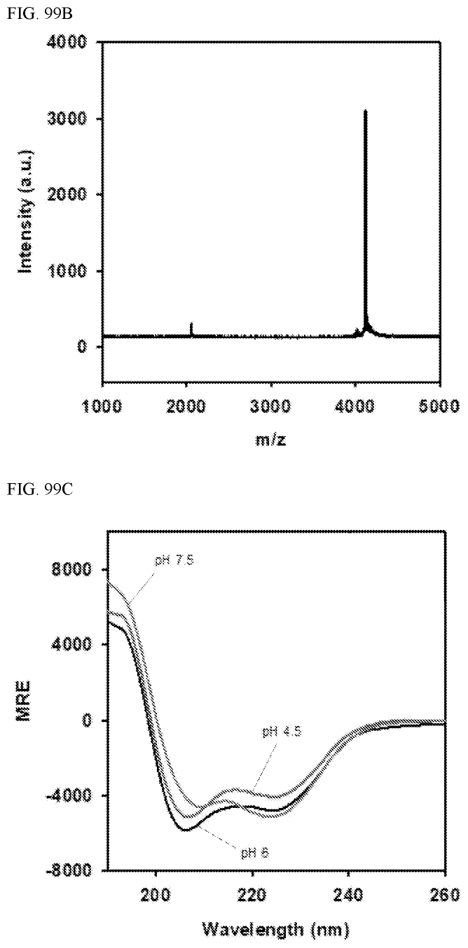

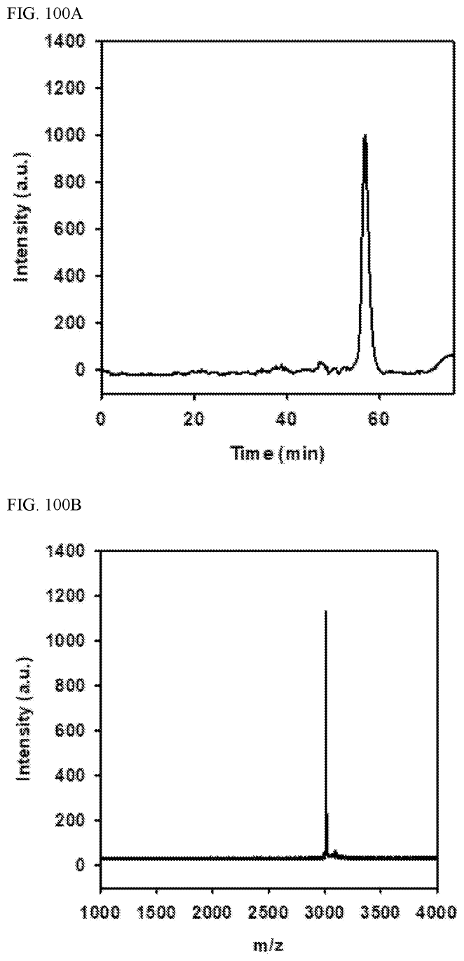

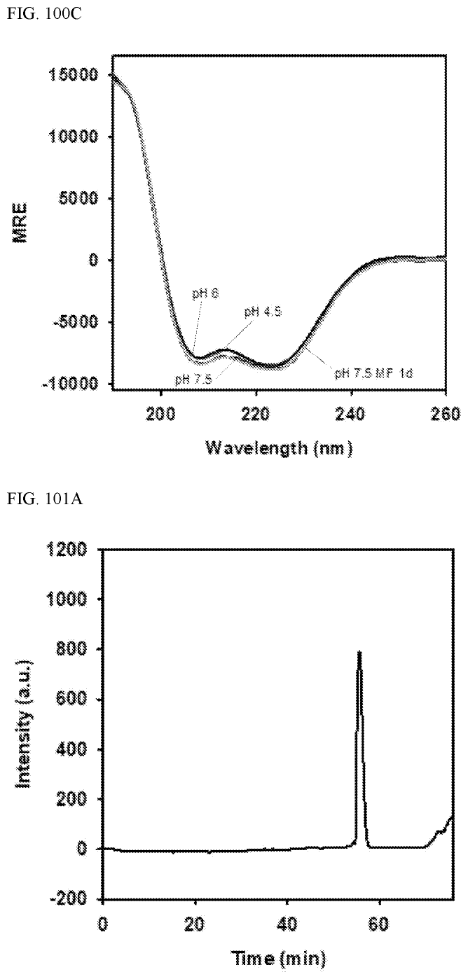

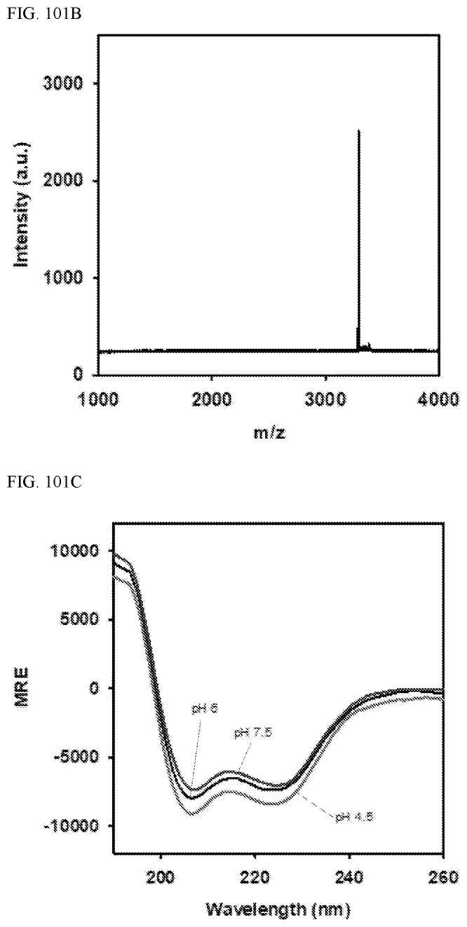

FIGS. 92A to 101C show the HPLC result (a), MALDI-TOF result (b) and CD spectra (c) of the cyclic peptide complexes of Examples 98-103, 106 and 109-111. FIG. 95D show the AFM image of the cyclic peptide complex of Example 101.

Table 13 shows the result of analyzing the morphological characteristics of the cyclic peptide complexes of Examples 98-111 by CD spectroscopy.

TABLE 13

| Name | Average molecular weight (g/mol) | [θ]₂₂₂/[θ]₂₀₈ |
|---|---|---|
| Example 98 | 1962 | n.d.^{a} |
| Example 99 | 2118.46 | 0.93 |
| Example 100 | 2274.64 | 0.95 |
| Example 101 | 2430.83 | 1.01 |
| Example 102 | 2587.01 | 1.10 |
| Example 103 | 2743.20 | 1.21 |
| Example 104 | | 0.2 |
| Example 105 | | 0.38 |
| Example 106 | 3650 | 0.65 |
| Example 107 | | 0.71 |
| Example 108 | | 0.83 |
| Example 109 | 4119 | 0.89 |
| Example 110 | 3013.41 | 1.06 |
| Example 111 | 3293.88 | 0.95 |

^{a}n.d. = not determined

As shown in FIGS. 92A-101C and Table 13, it was confirmed that the cyclic peptide complexes of Examples 98-103, 106 and 109-111 were synthesized successfully. It can be seen that the content of α-helices increases gradually as the length of the α peptide in the cyclic peptide complex increases. It was confirmed that the cyclic peptide complexes (Examples 98, 104 and 105) are unstable with the α-helical propensity lower than 0.5 when the number of the repeat unit [Aib]-[Xaa1] or [Xaa1]-[Aib] (hereinafter, also referred to as a) is smaller than 6. Specifically, it was confirmed that the cyclic peptide complexes of Examples 99-103 and 106-111 having 6-10 repeat units have the most stable α-helix structure.

### Test Example 18. Analysis of Biostability of Peptide Complexes

Through the experiments described above, the self-assembly behaviors of the peptide complex according to the present disclosure and the nanostructure with an artificial chromosome-like structure prepared therefrom were elucidated and their interactions with genetic materials were investigated to confirm their potential as genetic material storage media and systems. For stable storage of genetic materials and use in the fields of cells, medicine and foods, cytotoxicity is also of great importance. Accordingly, the cytotoxicity of the peptide complex according to the present disclosure and the nanostructure with an artificial chromosome-like structure prepared therefrom were analyzed.

For investigation of cytotoxicity, WST-1 assay was performed using Hela cells. First, Hela cells were subcultured in DMEM supplemented with 10% FBS (fetal bovine serum). After dispensing the cells in each well at 5×0⁴ Hela cells/well and culturing for 16 hours, they were treated with samples of various concentrations (5, 10 and 20 μM) and cultured at 37° C. for 24 hours. After adding WST-1 solution to the cultured cells, absorbance was measured at 450 nm 4 hours later using a microplate reader (Bio-Tek instrument Co., WA, USA).

The peptide complexes of Examples 40, 60 and 102 were prepared as the samples. In addition, as in Test Example 13 and Test Example 14, peptide-DNA nanostructures were prepared by mixing the peptide complexes of Examples 60 and 63 with linear plasmid DNA (artificial chromosome form, incubation for 12 days). Untreated normal cells were used as a control group.

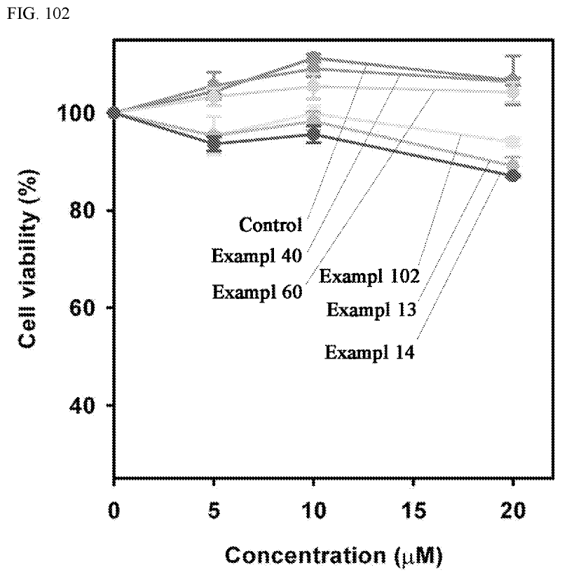

FIG. 102 shows a result of analyzing the cell viability for the peptide complexes of Examples 40, 60 and 102 and the nanostructures with an artificial chromosome-like structure prepared in Test Examples 13 and 14. It was confirmed that the peptide complexes and the nanostructures with various structures prepared according to the present disclosure are stable in the cells without causing cytotoxicity.

### Test Example 19. Structural Analysis of Peptide Complexes with Coil-Rod-Coil Structure

The peptide complexes of Examples 112-128 have a coil-rod-coil structure. The peptide complexes were purified by HPLC and analyzed by MALDI-TOF and CD spectroscopy. The HPLC condition was as follows: C4 semiprep TFA ACN 15-35% 10-60 min, mobile phase: ACN (TFA 0.1%), gradient: 15-35%/10-60 min (+0.4%/min), flow rate: 2.000 mL/min, injection volume: 4.5 mL, injection solvent: ACN 5%, temperature: 25° C., UV detection: 230 nm.

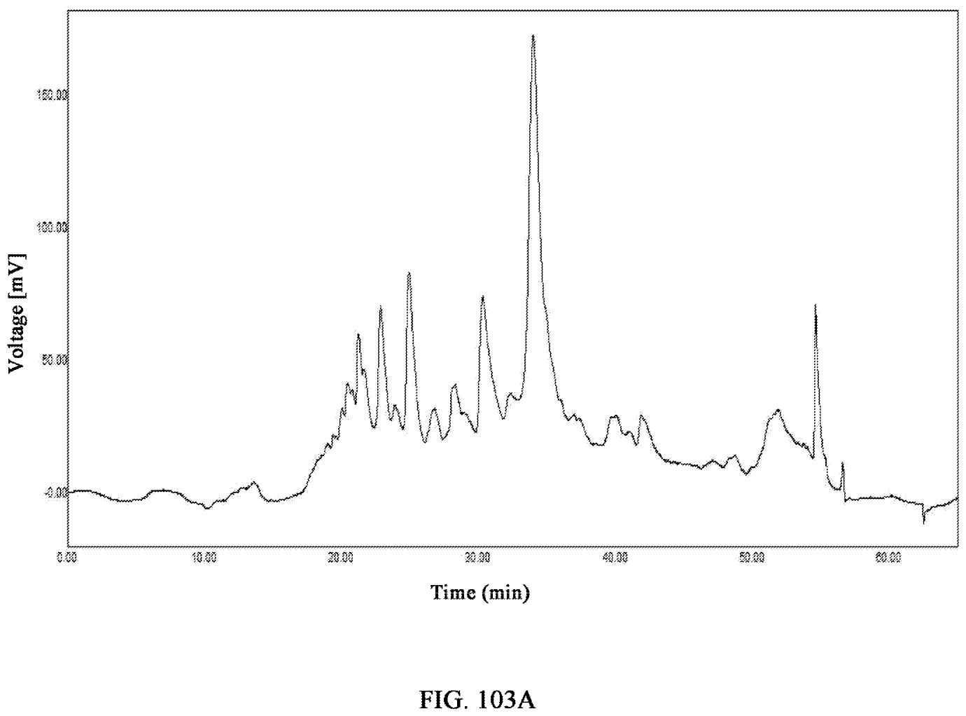

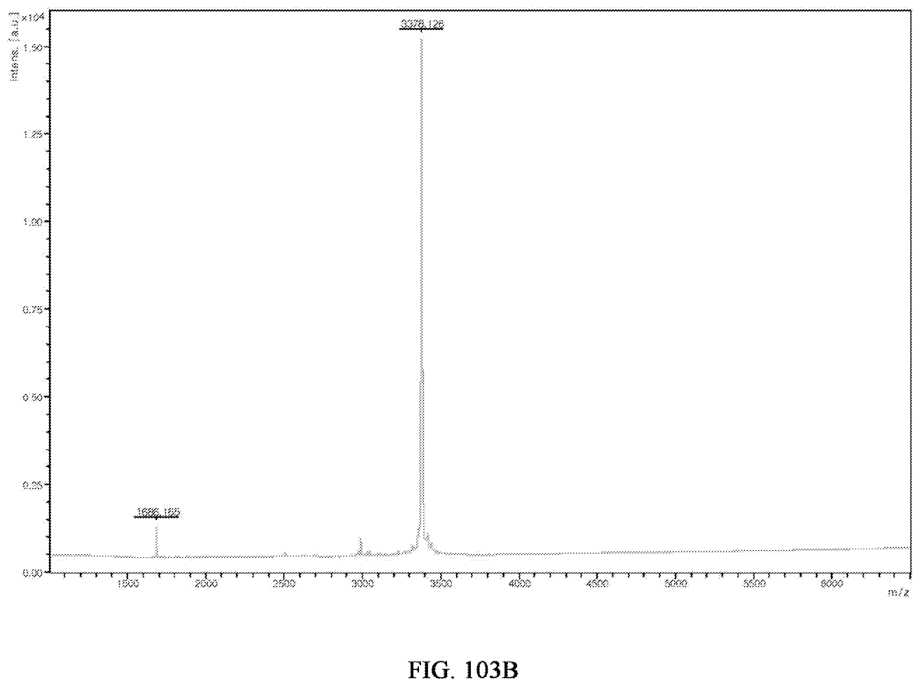

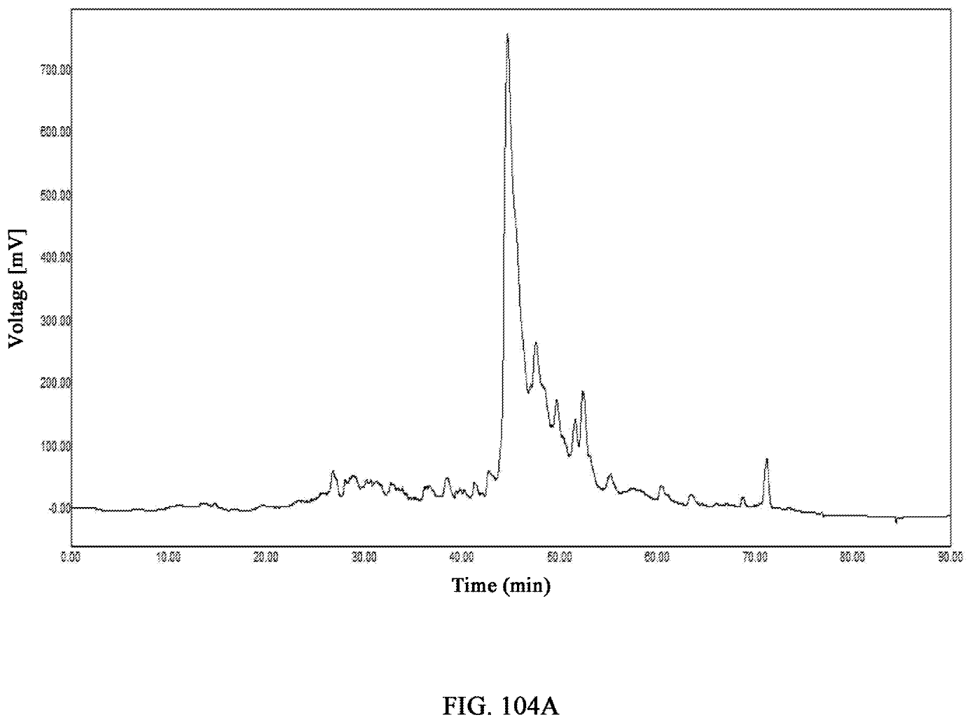

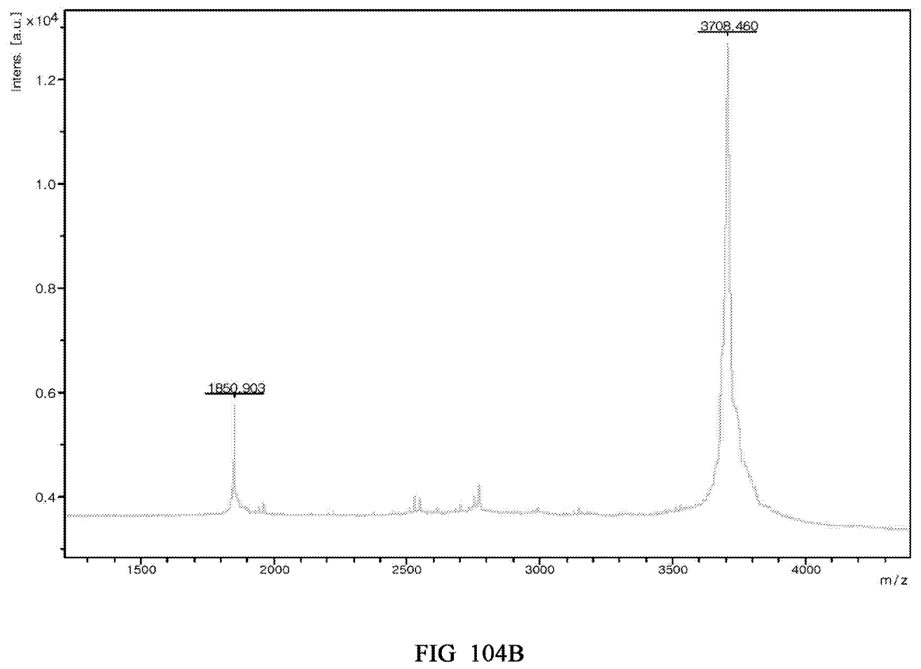

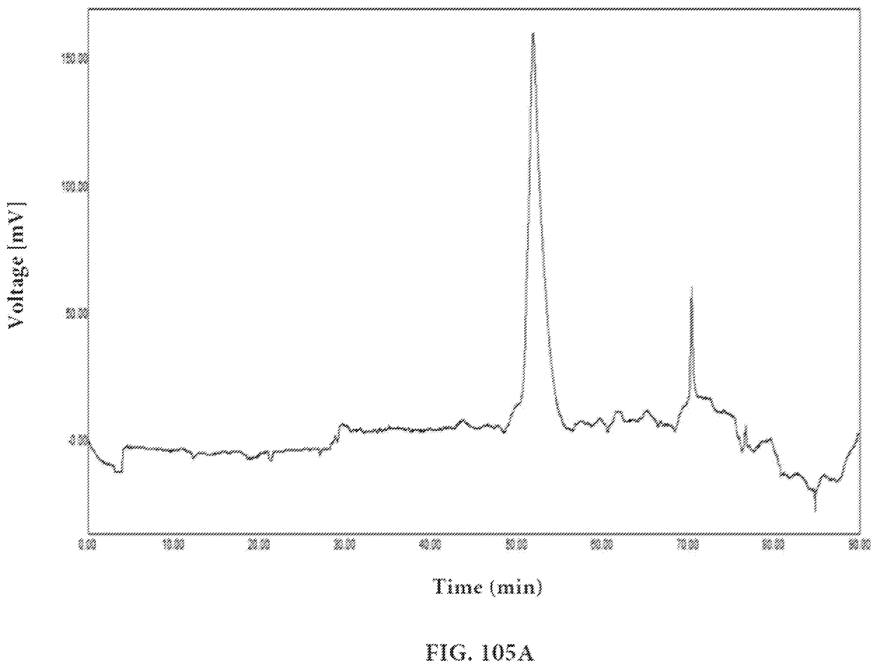

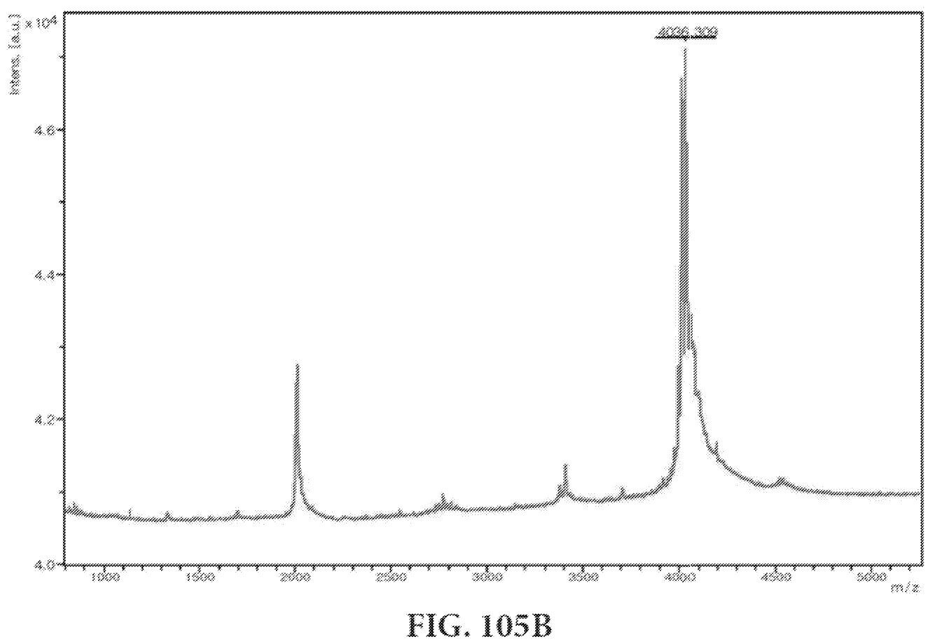

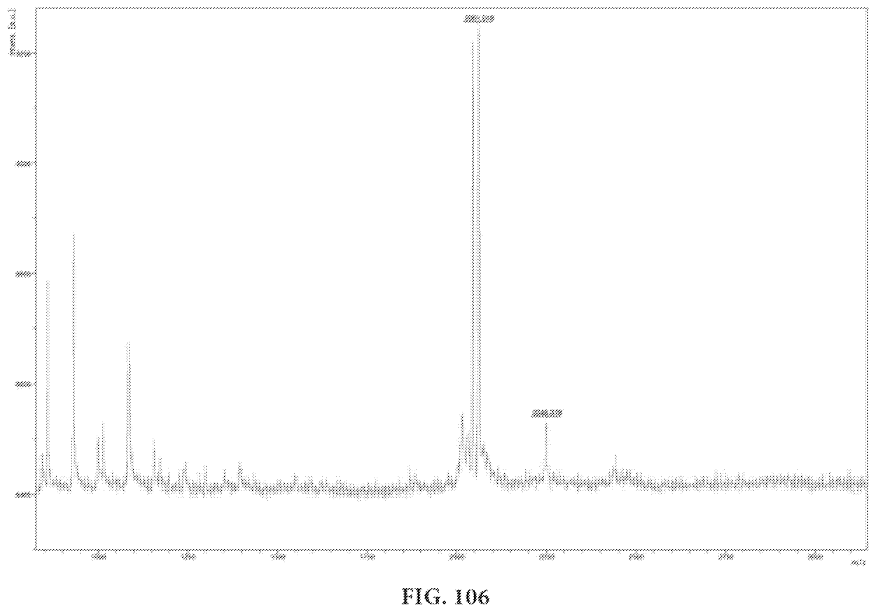

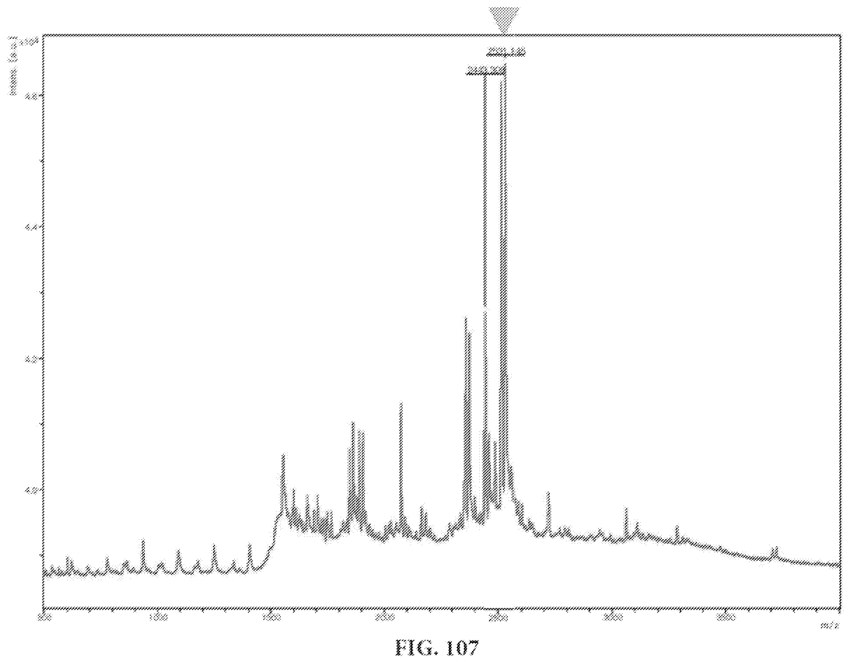

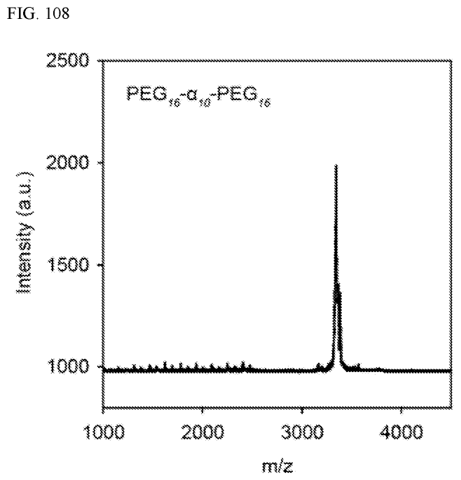

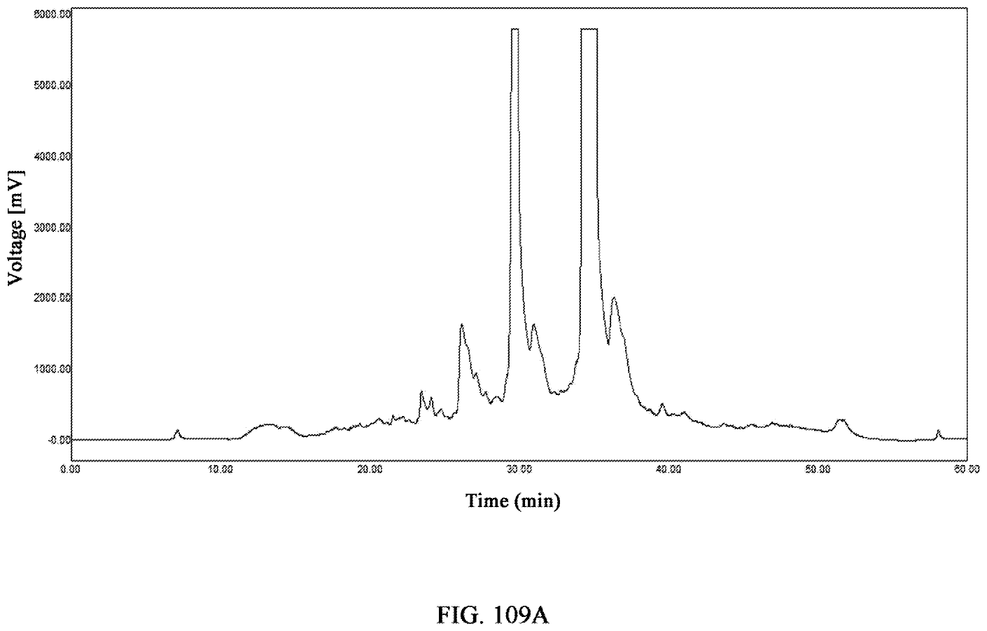

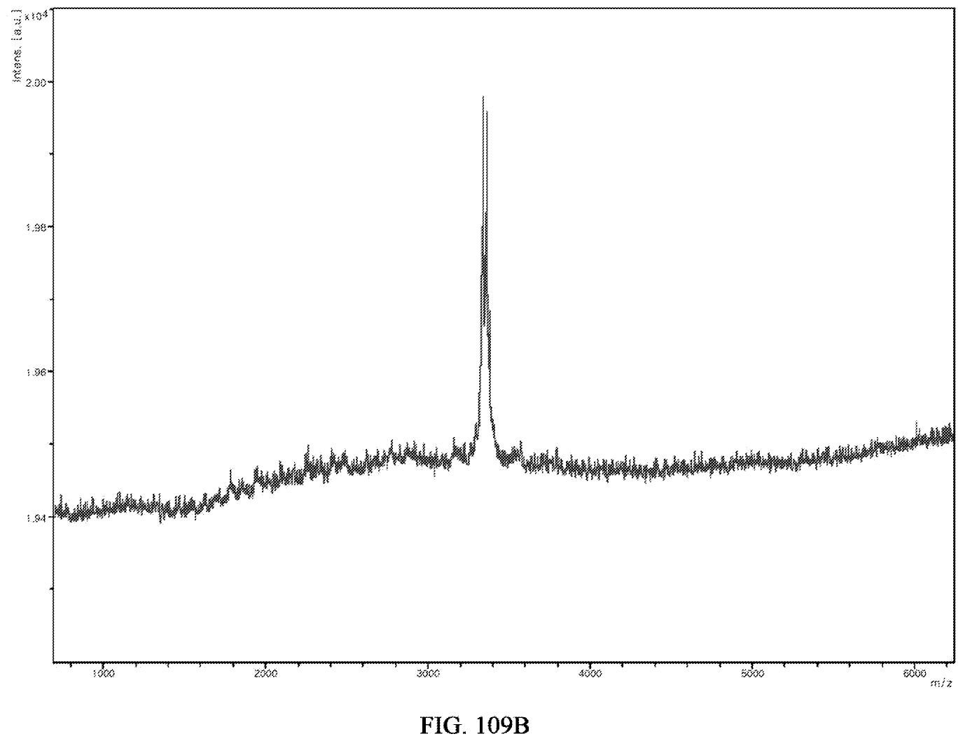

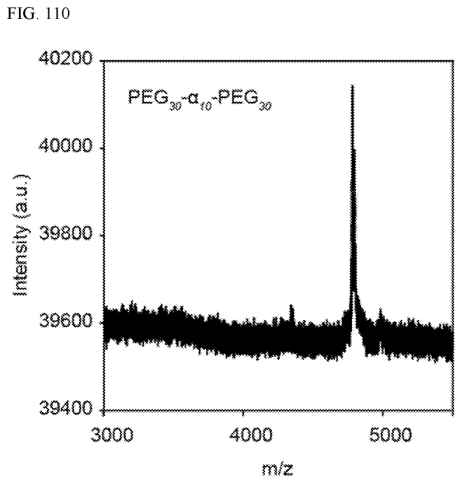

FIGS. 103A to 110 show the HPLC result (a) and MALDI-TOF result (b) for the peptide complexes of Examples 113-115, 119-121, 125 and 128. Table 14 shows the result of analyzing the morphological characteristics of the peptide complexes of Examples 116-128 by CD spectroscopy

TABLE 14

| | [θ]₂₂₂/[θ]₂₀₈ |
|---|---|
| Example 116 | 1.15 |
| Example 117 | 1.07 |
| Example 118 | 1.02 |
| Example 119 | 0.61 |
| Example 120 | 0.83 |
| Example 121 | 1.04 |
| Example 122 | 1.06 |
| Example 123 | 0.6 |
| Example 124 | 0.78 |
| Example 125 | 1.21 |
| Example 126 | 1.18 |
| Example 127 | 1.2 |
| Example 128 | 1.15 |

As shown in FIGS. 102-110 and Table 14, it was confirmed that the peptide complexes of Example 112-128 having a coil-rod-coil structure were synthesized successfully. It can be seen that the content of α-helices increases gradually as the length of the α peptide in the cyclic peptide complex increases. It was confirmed that the cyclic peptide complexes (Examples 98, 104 and 105) are unstable with the α-helical propensity lower than 0.5 when the number of the repeat unit [Aib]-[Xaa1] or [Xaa1]-[Aib] (hereinafter, also referred to as ‘α’) is smaller than 6. Specifically, it was confirmed that the cyclic peptide complexes of Examples 112-118, 120-122 and 124-128 having 6-10 repeat units have the most stable α-helix structure.

What is claimed is:

1. A hydrophobic peptide represented by General Formula 1 or 2:
-{[Aib]-[Xaa1]}ₘ- [General Formula 1]
-[Xaa2]n-{[Aib]-[Xaa1]}m- [General Formula 2]
wherein
Xaa1 is a D- or L-amino acid residue selected from a group consisting of Ala (A), Ile (I), Leu (L), Met (M) and Val (V), and
m is any integer selected from 4 to 50.

2. The hydrophobic peptide according to claim 1, wherein, in General Formula 1, Xaa1 is any one selected from a group consisting of Ala (A), Ile (I), Leu (L) and Met (M).

3. The hydrophobic peptide according to claim 1, wherein, in General Formula 1, Xaa1 is Ala (A) or Leu (L).

4. The hydrophobic peptide according to claim 1, wherein, in General Formula 1, m is any integer selected from 6 to 13.

5. The hydrophobic peptide according to claim 1, wherein, in General Formula 1, m is any integer selected from 6 to 10.

6. The hydrophobic peptide according to claim 1, wherein the hydrophobic peptide is a rod-shaped hydrophobic peptide having an α-helix structure.

7. The hydrophobic peptide according to claim 1, wherein the hydrophobic peptide is controlled to be arranged and oriented in the direction of an external magnetic field.

8. A peptide complex comprising:
the hydrophobic peptide according to claim 1; and
at least one hydrophilic polymer or hydrophilic peptide bound to one or both ends of the hydrophobic peptide.

9. The peptide complex according to claim 8, wherein the hydrophilic polymer is any one selected from a group consisting of polyethylene glycol (PEG), poly(N-(2-hydroxypropyl)methacrylamide), poly(2-(methacryloyloxy)ethyl phosphorylcholine), poly(hydroxyalkyl L-asparagine) and poly(hydroxyalkyl L-glutamine).

10. The peptide complex according to claim 8, wherein the hydrophilic peptide consists of 3-20 amino acid sequences, wherein 70-100% of the amino acid sequences are hydrophilic amino acids, and the hydrophilic amino acid is one or more selected from a group consisting of H (histidine), N (asparagine), Q (glutamine), S (serine), T (threonine), C (cysteine), G (glycine), K (lysine), R (arginine), E (glutamic acid) and D (aspartic acid).

11. The peptide complex according to claim 8, wherein the hydrophilic peptide is any one selected from a group consisting of SEQ ID NOS 98-105.

12. The peptide complex according to claim 8, wherein the peptide complex is cyclic with both ends of the peptide complex linked.

13. The peptide complex according to claim 8, wherein the peptide complex further comprises a positively charged peptide consisting of positively charged 1-4 amino acid residues at the N- or C-terminal.

14. The peptide complex according to claim 13, wherein the positively charged peptide is any one selected from SEQ ID NOS 139-150.

15. The peptide complex according to claim 8, wherein the peptide complex self-assembles into a spherical nanoparticle such as a micellar structure or a vesicular structure in solution.

16. The peptide complex according to claim 8, wherein the peptide complex self-assembles into an anisotropic planar nanostructure or a cruciform anisotropic nanostructure under the condition where a magnetic field is applied.

17. The peptide complex according to claim 16, wherein the intensity of the magnetic field is 0.1-2 T.

18. A composition for safely storing nucleic acid information, comprising: the peptide complex according to claim 8; and a nucleic acid material.

19. The composition according to claim 18, wherein the peptide complex and the nucleic acid material self-assemble into a nanoribbon or a nanostructure having an artificial chromosome-like structure via noncovalent bonding.

20. The composition according to claim 18, wherein a nanoribbon structure is formed through self-assembly as the peptide complex binds to the nucleic acid material and a nanostructure with an artificial chromosome-like structure is formed through self-assembly as the nanoribbon is folded and stacked.

21. The composition according to claim 18, wherein the nucleic acid material is one or more selected from a group consisting of an RNA, a DNA, an siRNA (short interfering RNA), an aptamer, an antisense ODN (oligodeoxynucleotide), an antisense RNA, a ribozyme and a DNAzyme.

22. The composition according to claim 20, wherein the nanostructure is degraded at a magnetic field intensity of 0.1-2 T and induces the release of the nucleic acid material.

23. The composition according to claim 22, wherein the magnetic field intensity is 0.1-0.5 T based on a rotating magnetic field.
